(19) United States
(12) Patent Application Publication
(10) Pub. No.: US 2006/0210537 A1
(43) Pub. Date: Sep. 21, 2006

(54) Theileria parva DNA vaccines

(76) Inventors: Jean-Christophe Francis Audonnet, Lyon (FR); Sheena May Loosmore, Aurora (CA)

Correspondence Address: FROMMER LAWRENCE & HAUG, 745 FIFTH AVENUE- 10TH FL., NEW YORK, NY, 10151, US

(21) Appl. No.: 11/184,236

(22) Filed: Jul. 19, 2005

Related U.S. Application Data
(60) Provisional application No. 60/662,646, filed on Mar. 17, 2005.

(51) Int. Cl. A61K 48/00; C07H 21/04; C12N 15/86

(52) U.S. Cl. 424/93.2; 536/23.7; 435/456

(57) ABSTRACT

The present invention relates to recombinant anti-Theileria parva vaccines and the administration of such vaccines to animals, advantageously bovines. Advantageously, the anti-Theileria parva vaccine encompasses a recombinant avipox virus that includes a nucleotide sequence encoding a Theileria parva gene. The invention further relates to methods of vaccinating animals, advantageously bovines, by administration of anti-Theileria parva vaccines that may encompass a recombinant avipox virus that may contain a Theileria parva gene.

### INCORPORATION BY REFERENCE

This application claims priority form U.S. Provisional Patent Application Ser. No. 60/662,646 filed Mar. 17, 2005 and incorporated herein by reference in its entirety. All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### SEQUENCE LISTING

The instant application contains a “lengthy” Sequence Listing which has been submitted via CD-R in lieu of a printed paper copy, and is hereby incorporated by reference in its entirety. Said CD-R, recorded on Jul. 15, 2005, are labeled CRF, “Copy 1” and “Copy 2”, respectively, and each contains only one identical 10.2 Mb file (57432681.APP).

### FIELD OF THE INVENTION

The present invention relates to vectors encompassing at least one polynucleotide of Theileria parva or at least one nucleic acid molecule encoding at least one Theileria parva antigen, immunogen or epitope, e.g., in vivo and in vitro expression vectors comprising and expressing at least one polynucleotide of the Theileria parva or in vivo and in vitro expression vectors comprising and expressing at least one Theileria parva antigen, immunogen or epitope. The present invention also relates to immunogenic compositions and vaccines against East Coast fever; for instance, such compositions or vaccines that contain one or more of the vectors and/or one or more of the expression products of the vectors. The invention further relates to methods for using the vectors, compositions and vaccines, including immunizing and vaccinating against Theileria parva, expressing expression products of the polynucleotide(s), using the expression products in assays or to generate antibodies useful in assays, as well as to methods for making the polynucleotide(s), vectors, compositions vaccines, assays, inter alia.

### BACKGROUND OF THE INVENTION

Theilerioses are a group of disease syndromes affecting cattle, sheep, goats and domestic buffalo caused by tick-borne haemo-protozoan parasites in the genus Theileria. The most economically important diseases include Mediterranean fever, East Coast Fever (ECF) and malignant theileriosis. Mediterranean fever caused by Theileria annulata occurs in North Africa, southern Europe, Near East, Middle East and many parts of Far East Asia with a population of 200 million cattle and buffalo at risk. ECF, caused by T. parva, affects 30 million cattle in eastern, central and southern Africa. Malignant theileriosis caused by T, lestoquardi affects sheep and goats in southeastern Europe, North Africa, the Near and Middle East and southern Russia and neighbouring States. These parasites belong to the same api-complexan group as Plasmodium falciparum, Toxoplasma gondii, Cytoxauzoon spp, Eimeria spp and Babesia spp, with a life-cycle having arthropod and mammalian components in which sexual and asexual stages develop, respectively. The pathogenic stages of Theileria parasites differ. T. parva causes a lympho-proliferative disorder in which schizont-infected lymphoblasts are responsible for the pathogenesis of the disease. Anaemic disease caused by T. lestoquardi and T. sergenii is due to piroplasm-infected erythrocytes while both the schizont and piroplasm of T. annulata are pathogenic resulting in lympho-proliferative and anaemic syndromes, respectively.

Currently, theilerioses are controlled largely by tick eradication using acaricides and through “infection and treatment” vaccination protocols of animals at risk. Such vaccination protocols are deemed effective in that an animal vaccinated in this manner will not develop ECF disease upon exposure to infectious T. parva. Due to cost and problems of tick resistance and environmental pollution, control of these diseases through acaricidal destruction of ticks is not sustainable.

Vaccination, while effective, presents shortcomings associated with the use of live vaccines. Owing to the ease of transmission of T. annulata, infected blood was originally used to immunise cattle by employing parasites of low virulence as the immunizing agent. However, such immunisations were accompanied by clinically-detectable infection episodes. With the advent of in vitro cultivation of T. annulata and the development of bulk culture techniques in the 1960s, significant progress was made in realising a more practical immunisation strategy. Currently, passage-attenuated cultures of T. annulata are routinely used in national vaccination programs in affected countries. By contrast, similar efforts to immunise cattle against T. parva have been unsuccessful, attributable to the failure of attenuated T. parva parasites to induce immunity. Much higher numbers of T. parva-infected cells are required to infect cattle reliably, since the schizonts of T. parva transfer at a low frequency and donor cells are rejected before successful transfer. T. lestoquardi has also been cultivated in vitro and studies have shown that attenuated parasites can be used to immunise animals with a degree of success.

Given the unsuccessful attempts to immunise cattle with attenuated T. parva subsequent efforts have focused on the use of virulent parasites with accompanying chemotherapy. The rationale of this infection and treatment method (ITM) is to allow the infection to establish and suppress development of frank clinical disease by administering theileriacidal drugs. Animals thus immunised are protected against the development of disease when exposed to the homologous parasite. This vaccination strategy has undergone successive refinements including the use of cryopreserved triturated tick stabilites containing sporozoites (the parasite stage infective for cattle lymphocytes) to standardise the immunisation-infection dose, as well as simultaneous drug administration. Further improvement of this immunisation approach has involved the identification and use of a combination of parasite stocks to broaden the immunising spectrum of the vaccine against several field T. parva parasite populations. The use of local parasite stocks to immunise in areas where they have been isolated is also practised. ITM immunisation against T. parva has been tested extensively under laboratory and field conditions and is now deployed in the affected region to control ECF.

ITM has a number of practical limitations that hinder its application as a sustainable control measure against ECF in those geographical areas most affected by the disease. Being live, it requires a cold refrigerator chain, which is impractical in Africa. ITM can also cause clinical disease if drug application is inadequate and it has the potential to introduce new parasite strains in areas under the vaccination campaign. The cost (US$ 10-20 per immunisation) of this current vaccine is well beyond the budget of poor farmers with cattle afflicted by ECF. Because of these concerns associated with the ITM vaccine protocol, there is still a need for a vaccine that will be sustainable and affordable.

A 67 kDa glycoprotein (p67) from the surface of the T. parva sporozoite has been isolated (U.S. Pat. No. 5,273,744) and used in a variety of immunization protocols, with little success reported so far in the development of practical levels of immune-mediated disease resistance. However, cattle recovering from a single infection with T. parva sporozoites resist infection upon homologous challenge. Such animals have weak antibody and T cell responses to p67. There is still a need, therefore, to identify T. parva antigens that can induce antigen-specific class I MHC-restricted CD8⁺ cytotoxic T lymphocytes (CTLs).

Accordingly, there is a need in the art for an efficacious and reliable Theileria parva vaccine where heterologous proteins are expressed with limited or no productive replication.

Citation or identification of any document in this application is not an admission that such document is available as prior art to the present invention.

### SUMMARY OF THE INVENTION

The present invention provides vectors containing at least one polynucleotide of the Theileria parva or at least one nucleic acid molecule encoding at least one Theileria parva antigen, immunogen or epitope, e.g., in vivo and in vitro expression vectors comprising and expressing at least one polynucleotide of the Theileria parva. The present invention further encompasses in vivo and in vitro expression vectors comprising and expressing at least one Theileria parva antigen, immunogen or epitope, as well as immunogenic compositions and vaccines against East Coast fever. Such compositions or vaccines may contain one or more of the vectors and/or one or more of the expression products of the vectors.

The present invention encompasses any immunogen or antigen of T. parva including, but is not limited to, any of the polypeptides Tp 1-8, fragments thereof, or combinations thereof. The combinations may be separate proteins or polyproteins. The compositions or vaccines may thus contain one or more vectors expressing more than one of the polypeptides, e.g., different proteins, immnogens or antigens. The compositions or vaccines may contain, or vectors thereof express, polypeptides from different strains or isolates of Theileria parva. Thus, the compositions or vaccines may contain, or the vectors thereof may express, but are not limited to, Tp1-8, fragments thereof, or combinations thereof, wherein the polypeptides Tp1-8 are from different strains or isolates.

Advantageously, in embodiments involving at least one epitope present in, or expressed by, a vector or vectors in compositions or vaccines of the invention, the epitope or epitopes may be from, but not limited to, any of Tp1-8 polypeptides of Theileria parva, fragments thereof, or combinations thereof, and the epitope or epitopes can be from different strains or isolates. In this regard, it is noted that one of skill in the art can locate or map epitopes in Theileria parva antigens or immunogens, such as the Tp1.

The invention further provides methods for using the vectors, compositions and vaccines, including immunizing and vaccinating against T. parva, expressing expression products of the polynucleotide(s), and methods for using the expression products in assays or to generate antibodies useful in assays, as well as to methods for making the, polynucleotide(s), vectors, compositions vaccines, assays, inter alia.

The present invention thus relates to means for preventing and/or combating diseases caused by the Theileria parva. The invention relates to such immunogenic and vaccine compositions suitable for use in different animal (target or host) species susceptible to disease caused by Theileria parva, in particular bovines, and the like.

The invention further provides immunization and vaccination methods involving the immunogenic and vaccine compositions of the invention, for the target or host species. And on this aspect of the invention, mention is made of compositions comprising one or more vectors that express one or more Theileria parva epitopes or antigens or immunogens can be delivered via food, e.g., cattle feed for consumption by wild or non-domesticated bovines, that includes or contains the one or more vectors. This route of administration may be advantageous when the one or more vectors is one or more poxviruses, e.g., an avipox virus such as an attenuated canarypox virus, for instance ALVAC, or an attenuated fowlpox virus, for instance TROVAC, or a vaccinia virus, such as an attenuated vaccinia virus, for instance NYVAC. Accordingly, the invention envisions oral or mucosal administration, as well as injectable compositions that contain one or more of the inventive vectors. From this disclosure and the knowledge in the art, the skilled artisan can formulate edible animal feed for a mammal that contains a suitable dose of one or more inventive vectors.

The invention further provides means and methods that make differential diagnosis possible, e.g., methods that make it possible to make, or allow for, a distinction between an animal infected by the East Coast fever pathogen and an animal administered a vaccine or immunogenic composition according to the invention.

In certain embodiments, the invention provides in vitro and/or in vivo expression vectors which may comprise a polynucleotide encoding any of, but not limited to, the proteins Tp1-8 of Theileria parva and the like, or an immunogenic fragment of such a protein. These vectors advantageously also may comprise the elements for the expression of the polynucleotide in a host cell.

In addition to the polynucleotide encoding any of Tp1-8 a fragment thereof, and the like, the expression vectors according to the invention may comprise one or more other polynucleotides encoding other proteins of the Theileria parva, advantageously structural proteins of the Theileria parva and said sequences are advantageously chosen from among those encoding membrane proteins, sporocyst antigens, transmembrane proteins and the like.

The vectors of the invention may advantageously comprise a polynucleotide forming a single encoding frame or coding region corresponding to, but not limited to, any of Tp1-8, or epitopes thereof; that is, expression of a polyprotein or epitopes thereof are considered advantageous. A vector comprising several separate polynucleotides encoding the different proteins (e.g. any of the Tp1-8 proteins) or epitopes thereof also falls within the scope of the present invention. The vector, especially for in vivo expression, may also comprise polynucleotides corresponding to more than one Theileria parva strain or isolate, for instance, two or more polynucleotides encoding Tp1-8, or epitope(s) thereof, of different strains.

Likewise, an immunogenic or vaccine composition may comprise one or more vectors for expression of polynucleotides corresponding to more than one Theileria parva strain or isolate. The vector, especially for in vivo expression, can additionally comprise one or more nucleotide sequences encoding immunogens of other pathogenic agents and/or cytokines. According to a preferred embodiment of the invention, the expression vectors of the present invention may comprise a polynucleotide encoding Tp1-8 or a fragment thereof, and preferably in a single reading frame.

One aspect of the present invention, therefore, is an avipox expression vector that may comprise a polynucleotide that encodes a Theileria parva immunogen. Advantageously, the T. parva poylpeptide may be an immunogen capable of inducing an immune response in an animal such as a bovine. In one embodiment of this aspect of the invention, the Theileria parva immunogen may be a polypeptide selected from the group consisting of Tp1, Tp2, Tp3, Tp4, Tp5, Tp6, Tp7 and Tp8. In various embodiments of the invention, the Theileria parva immunogen is a peptide having an amino acid sequence according to any of the group consisting of SEQ ID NOs: 7, 16, 26, 30, 32, 39, 44-49, 60-64 or a fragment thereof.

In other embodiments of the invention, the polynucleotide may comprise a nucleotide base sequence according to any of the group consisting of SEQ ID NOs: 1, 6, 12, 15, 17, 19, 24, 25, 29, 33, 36, 38, 43, 50-59, or the complement thereof.

In the various embodiments of the invention, the avipox expression vector is an attenuated avipox expression vector. In one embodiment, the avipox expression vector is a canarypox vector. In an advantageous embodiment the canarypox vector is ALVAC.

In yet other embodiments of this aspect of the invention, the avipox expression vector is a fowlpox vector. In one advantageous embodiment, the fowlpox vector is TROVAC.

Another aspect of the invention is a formulation for delivery and expression of a Theileria parva immunogen, wherein the formulation comprises an avipox expression vector comprising a polynucleotide that encodes a Theileria parva immunogen, and a pharmaceutically or veterinarily acceptable carrier, vehicle or excipient.

In this aspect of the invention, one embodiment further comprises an attenuated vaccinia virus comprising a polynucleotide comprises a nucleotide base sequence according to any of the group consisting of SEQ ID NOs: 1, 6, 12, 15, 17, 19, 24, 25, 29, 33, 36, 38, 43, 50-59, or the complement thereof. In another embodiment, the attenuated vaccinia virus is NYVAC.

Yet another aspect of the invention is a formulation for delivery and expression of a Theileria parva immunogen, wherein the formulation comprises an avipox expression vector comprising a nucleotide base sequence according to any of the group consisting of SEQ ID NOs: 1, 6, 12, 15, 17, 19, 24, 25, 29, 33, 36, 38, 43, 50-59, or the complement thereof, and wherein the avipox expression vector is an attenuated avipox expression vector, a canarypox vector, a fowlpox vector or an attenuated vaccinia virus. In one embodiment of this aspect, the fowlpox vector is TROVAC. In still another embodiment, the canarypox vector is ALVAC. In yet another embodiment, the attenuated vaccinia virus is MVA.

Another aspect of the invention is a method of delivering a Theileria parva antigen to an animal, comprising administering to the animal a formulation for delivery and expression of a Theileria parva immunogen, wherein the formulation comprises an avipox expression vector comprising a nucleotide base sequence according to any of the group consisting of SEQ ID NOs: 1, 6, 12, 15, 17, 19, 24, 25, 29, 33, 36, 38, 43, 50-59, or the complement thereof, and wherein the avipox expression vector is an attenuated avipox expression vector, a canarypox vector, a fowlpox vector or an attenuated vaccinia virus.

One embodiment of the invention is a method of eliciting an immune response against a strain of Theileria in an animal, comprising administering a formulation for delivery and expression of a Theileria parva immunogen, wherein the formulation comprises an avipox expression vector comprising a nucleotide base sequence according to any of the group consisting of SEQ ID NOs: 1, 6, 12, 15, 17, 19, 24, 25, 29, 33, 36, 38, 43, 50-59, or the complement thereof, and wherein the avipox expression vector is an attenuated avipox expression vector, a canarypox vector, a fowlpox vector or an attenuated vaccinia virus and in an effective amount for eliciting an immune response.

Still another embodiment of the invention is a method of inducing an immunological or protective response against a strain of Theileria in an animal, comprising administering to the animal an effective amount of a formulation for delivery and expression of a Theileria parva immunogen, wherein the formulation comprises an avipox expression vector comprising a polynucleotide that encodes a Theileria parva immunogen, and a pharmaceutically or veterinarily acceptable carrier, vehicle or excipient.

A method of eliciting an immune response against a strain of Theileria in an animal, comprising administering a composition comprising a cell, wherein the cell comprises an avipox expression vector comprising a polynucleotide that encodes a Theileria parva immunogen.

Another aspect of the invention is a kit for performing a method of inducing an immunological or protective response against a strain of Theileria in an animal comprising an avipox expression vector comprising a nucleotide base sequence that encodes a Theileria parva immunogen, or the complement thereof, and wherein the avipox expression vector is an attenuated avipox expression vector, a canarypox vector, a fowlpox vector or an attenuated vaccinia virus, and instructions for performing the method of delivery and expression of the vector in the animal.

Another embodiment of the invention is a kit for performing a method of inducing an immunological or protective response against a strain of Theileria in an animal comprising an avipox expression vector comprising a nucleotide base sequence according to any of the group consisting of SEQ ID NOs: 1, 6, 12, 15, 17, 19, 24, 25, 29, 33, 36, 38, 43, 50-59, or the complement thereof, and wherein the avipox expression vector is an attenuated avipox expression vector, a canarypox vector, a fowlpox vector or an attenuated vaccinia vector and instructions for performing the method of delivery and expression of the vector in an effective amount for eliciting an immune response in the animal.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 illustrates the nucleotide sequence of Tp1 (SEQ ID NO 1) of plasmid pTarget Tp1, the amino acid sequence SEQ ID NO: 7 of the Tp1 polypeptide encoded therein. Nucleotide and amino acid substitutions compared to the nucleotide sequence SEQ ID NO: 51 (GenBank Accession No. CS008930) encoding the amino acid sequence SEQ ID NO 45 of Tp1 are boxed.

FIG. 2 illustrates schematically the construction of plasmid pSL-6174-1-1.

FIG. 3 illustrates the sequences of primers used in the construction of Tp-encoding clones, those regions of the oligonucleotides encoding amino acids and restriction sites within the primers useful for cloning (SEQ ID NOS: 2-5, 13-14, 20-21, 27-28, 31, 37 and 78-94).

FIG. 4 illustrates a structural map of the plasmid pSL-6174-1-1.

FIG. 5 illustrates the nucleotide sequence (SEQ ID NO 6) of the insert encoding Tp1 within pSL-6174-1-1.

FIGS. 6A-6M illustrates the amino acid sequences SEQ ID NOs: 7, 16, 26, 30, 32, 39, residues 263-459 of 44, 45-49 of the Theileria parva Tp-related polypeptides.

FIG. 7 illustrates schematically the construction of vector ALVAC Tp1 (vCP2112).

FIG. 8 illustrates the nucleotide sequence (SEQ ID NO: 12) of the insert encoding the polypeptide Tp1 of construct vCP2112.

FIG. 9 illustrates schematically the construction of the vector pSL-6104-5 having the Tp2 encoding polynucleotide inserted into vector PCXL-148-2.

FIG. 10 illustrates a map of vector pSL-5151-2.

FIG. 11 illustrates the nucleotide sequence SEQ ID NO: 15 of the insert within vector pSL-6151-2.

FIG. 12 schematically illustrates the in vivo recombination generating the ALVAC clone vCP2110 bearing the Tp2 encoding insert.

FIG. 13 illustrates the nucleotide sequence (SEQ ID NO: 95) and domains of the insert within plasmid pSL-6151-2 (Protein disclosed as SEQ ID NO: 16).

FIG. 14 illustrates nucleotide sequence SEQ ID NO: 65 of the CRL-H6p-Tp2-tag-CRR insert of the pSL-6151-2 vector.

FIG. 15 illustrates the nucleotide sequence SEQ ID NO: 17 of the C5R-H6p-Tp2-C5L insert of the vector vCP2110.

FIG. 16 illustrates the nucleotide sequence (SEQ ID NO: 19) of the Tp3-tag#5 insert of the plasmid pSG Tp3-tag#5 (Protein disclosed as SEQ ID NO: 26).

FIG. 17 schematically illustrates the construction of plasmid pCXL853.2.

FIG. 18 illustrates the nucleotide sequence SEQ ID NO: 24 of the insert of the plasmid construct pCXL853.2 (pC5H6-Tp3-tag).

FIG. 19 schematically illustrates the construction of the ALVAC vector vCP2143 with the H6p-Tp3-tag encoding insert.

FIG. 20 illustrates the nucleotide sequence SEQ ID NO: 25 of the H6p-Tp3-tag insert, polypeptide sequence translated therefrom (SEQ ID NO: 26), and flanking sequences inserted into the ALVAC vector vCP2143.

FIG. 21 schematically illustrates the construction of plasmid pSL-6555-2-1 having an H6p-Tp4-tag insert.

FIG. 22 illustrates the nucleotide sequence SEQ ID NO: 29 of the C5R H6p-Tp4-tag-C5L insert of the plasmid pSL-6555-2-1.

FIG. 23 schematically illustrates the construction of the plasmid pSL-6439-2 containing the insert encoding H6p-Tp5-tag.

FIG. 24 illustrates the nucleotide sequence SEQ ID NO: 33 of the inserted region of plasmid pSL-6439-2.

FIG. 25 schematically illustrates construction of the ALVAC vector vCP2138 bearing the insert Tp5-tag.

FIG. 26 illustrates the nucleotide sequence SEQ ID NO: 36 of the inserted region of the vector construct vCP2138.

FIG. 27 schematically illustrates construction of the plasmid vector pHM1103.1 containing the insert encoding H6p-Tp6-tag.

FIG. 28 illustrates the nucleotide sequence SEQ ID NO: 38 of the insert region encoding H6p-Tp6-tag, amino acid translations therefrom (SEQ ID NO: 39), and flanking sequences, of the plasmid vector pHM1103.1.

FIG. 29 illustrates the nucleotide sequence SEQ ID NO: 40 of the plasmid pHM1103.1.

FIG. 30 schematically illustrates construction of the ALVAC vector recombinant vCP2179 encoding the polypeptide Tp8-tag.

FIG. 31 illustrates the nucleotide sequence SEQ ID NO: 43 of the inserted region of vector vCP2179 encoding C5L-H6p-Tp8-tag-C5R and showing the amino acid sequence SEQ ID NO: 44 of the Tp8-tag polypeptide.

FIG. 32 illustrates the nucleotide sequence SEQ ID NO: 50 (GenBank Accession No. CS008930) encoding the amino acid sequence SEQ ID NO 45 of Tp1.

FIG. 33 illustrates the nucleotide sequence SEQ ID NO: 51 (GenBank Accession No. CS008931) encoding the amino acid sequence SEQ ID NO 46 of Tp4.

FIG. 34 illustrates the nucleotide sequence SEQ ID NO: 52 (GenBank Accession No. CS008932) encoding the amino acid sequence SEQ ID NO 47 of Tp5

FIG. 35 illustrates the nucleotide sequence SEQ ID NO: 53 (GenBank Accession No. CS008933) encoding the amino acid sequence SEQ ID NO 48 of Tp7.

FIG. 36 illustrates the nucleotide sequence SEQ ID NO: 54 (GenBank Accession No. CS008934) encoding the amino acid sequence SEQ ID NO: 49 of Tp8.

FIG. 37 illustrates the nucleotide sequences SEQ ID NOs: 55 (GenBank Accession No. CS00893), 56 (GenBank Accession No. CS008940), 57 (GenBank Accession No. CS008941), 58 (GenBank Accession No. CS008942), and 59 and their respective translation products SEQ ID NOs: 60-64 and derived from T. parva protein Tp1 (SEQ ID NO. 51).

FIG. 38 illustrates an immunoblot analysis of vCP2179, ALVAC-Tp8-tag expression.

FIG. 39 illustrates an immunoblot analysis of vFP2201 TROVAC-Tp2-tag expression.

FIG. 40 illustrates summed CD8⁺ T cell IFN-γ responses following MVA/CP immunization.

FIG. 41 illustrates summed CD8⁺ T cell IFN-γ responses following CP/MVA immunization.

FIG. 42 illustrates the individual kinetics of the IFNγ⁺CD8⁺ T cell response bovine BX223, ex-vivo stimulating antigen Tp8.

FIG. 43 illustrates the distribution of the BoLA type response after ex vivo individual antigen stimulation using PCA.

FIG. 44 illustrates the distribution of the BoLA type response.

### DETAILED DESCRIPTION

The practice of the present invention will employ, unless otherwise indicated, conventional techniques of molecular biology, microbiology, recombinant DNA technology, and immunology, which are within the skill of the art. Such techniques are explained fully in the literature. See, e.g., Sambrook, Fritsch & Maniatis, Molecular Cloning: A Laboratory Manual, Vols. I, II and III, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); and Handbook of Experimental Immunology, Vols. I-IV (D. M. Weir and C. C. Blackwell eds., 1986, Blackwell Scientific Publications).

Before describing the present invention in detail, it is to be understood that this invention is not limited to particular DNA, polypeptide sequences or process parameters as such may, of course, vary. It is also to be understood that the terminology used herein is for the purpose of describing particular embodiments of the invention only, and is not intended to be limiting.

The following amino acid abbreviations are used throughout the text: Alanine: Ala (A); Arginine: Arg (R); Asparagine: Asn (N); Aspartic acid: Asp (D); Cysteine: Cys (C); Glutamine: Gln (Q); Glutamic acid: Glu (E); Glycine: Gly (G); Histidine: His (H); Isoleucine: Ile (I); Leucine: Leu (L); Lysine: Lys (K); Methionine: Met (M); Phenylalanine: Phe (F); Proline: Pro (P); Serine: Ser (S); Threonine: Thr (T); Tryptophan: Trp (W); Tyrosine: Tyr (Y); and Valine: Val (V).

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed and are intended to be defined as indicated below.

The terms “protein”, “peptide”, “polypeptide” and “polypeptide fragment” as used herein refers to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

The term “polynucleotide” as used herein refers to a polymeric form of nucleotides of any length, which contain deoxyribonucleotides, ribonucleotides, and analogs in any combination. Polynucleotides may have three-dimensional structure, and may perform any function, known or unknown. The term “polynucleotide” includes double-, single-stranded, and triple-helical molecules. Unless otherwise specified or required, any embodiment of the invention described herein that is a polynucleotide encompasses both the double stranded form and each of two complementary forms known or predicted to make up the double stranded form of either the DNA, RNA or hybrid molecule.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support.

An “isolated” polynucleotide or polypeptide is one that is substantially free of the materials with which it is associated in its native environment. By substantially free, is meant at least 50%, advantageously at least 70%, more advantageously at least 80%, and even more advantageously at least 90% free of these materials.

An “isolated” nucleic acid molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith.

The term “polynucleotide encoding a protein” as used herein refers to a DNA fragment or isolated DNA molecule encoding a protein, or the complementary strand thereto; but, RNA is not excluded, as it is understood in the art that thymidine (T) in a DNA sequence is considered equal to uracil (U) in an RNA sequence. Thus, RNA sequences for use in the invention, e.g., for use in RNA vectors, can be derived from DNA sequences, by thymidine (T) in the DNA sequence being considered equal to uracil (U) in RNA sequences.

The term “protein” as used herein also includes proteins in neutral form or in the form of basic or acid addition salts depending on the mode of preparation. Such acid addition salts may involve free amino groups and basic salts may be formed with free carboxyls. In addition, the proteins may be modified by combination with other biological materials such as lipids and saccharides, or by side chain modification, such as acetylation of amino groups, phosphorylation of hydroxyl side chains, oxidation of sulfhydryl groups, glycosylation of amino acid residues, as well as other modifications of the encoded primary sequence.

The term “immunogenic protein or peptide” as used herein also refers includes peptides and polypeptides that are immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises or consists essentially of or consists of at least one epitope or antigenic determinant. The term epitope relates to a protein site able to induce an immune reaction of the humoral type (B cells) and/or cellular type (T cells).

The term “immunogenic protein or peptide” further contemplates deletions, additions and substitutions to the sequence, so long as the polypeptide functions to produce an immunological response as defined herein. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide.

The term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

The compositions of the invention can include any pharmaceutically acceptable carrier known in the art.

An “immunological response” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, suppressor-T cells, and/or cytotoxic T cells and/or γδ T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

The terms “immunogenic” protein or polypeptide as used herein also refers to an amino acid sequence which elicits an immunological response as described above. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Methods especially applicable to the proteins of T. parva are fully described in the PCT Application Serial No. PCT/US2004/022605 incorporated herein by reference in its entirety.

Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12th World AIDS Conference, Geneva, Switzerland, Jun. 28-Jul. 3, 1998. Immunogenic fragments, for purposes of the present invention, will usually include at least about 3 amino acids, preferably at least about 5 amino acids, more preferably at least about 10-15 amino acids, and most preferably 25 or more amino acids, of the molecule. There is no critical upper limit to the length of the fragment, which could comprise nearly the full-length of the protein sequence, or even a fusion protein comprising at least one epitope of the protein.

Accordingly, a minimum structure of a polynucleotide expressing an epitope is that it comprises or consists essentially of or consists of nucleotides to encode an epitope or antigenic determinant of the Theileria parva protein or polyprotein. A polynucleotide encoding a fragment of the total protein or polyprotein, more advantageously, comprises or consists essentially of or consists of a minimum of 21 nucleotides, advantageously at least 42 nucleotides, and preferably at least 57, 87 or 150 consecutive or contiguous nucleotides of the sequence encoding the total protein or polyprotein. As mentioned earlier, epitope determination procedures, such as, generating overlapping peptide libraries (Hemmer B. et al., Immunology Today, 1998, 19 (4), 163-168), Pepscan (Geysen et al., (1984) Proc. Nat. Acad. Sci. USA, 81, 3998-4002; Geysen et al., (1985) Proc. Nat. Acad. Sci. USA, 82, 178-182; Van der Zee R. et al., (1989) Eur. J. Immunol., 19, 43-47; Geysen H. M., (1990) Southeast Asian J. Trop. Med. Public Health, 21, 523-533; Multipin.RTM. Peptide Synthesis Kits de Chiron) and algorithms (De Groot A. et al., (1999) Nature Biotechnology, 17, 533-561), and in PCT Application Serial No. PCT/US2004/022605 all of which are incorporated herein by reference in their entireties, can be used in the practice of the invention, without undue experimentation. Other documents cited and incorporated herein may also be consulted for methods for determining epitopes of an immunogen or antigen and thus nucleic acid molecules that encode such epitopes.

“Native” proteins or polypeptides refer to proteins or polypeptides isolated from the source in which the proteins naturally occur. “Recombinant” polypeptides refer to polypeptides produced by recombinant DNA techniques; i.e., produced from cells transformed by an exogenous DNA construct encoding the desired polypeptide. “Synthetic” polypeptides include those prepared by chemical synthesis as well as the synthetic antigens described above.

Elements for the expression of the polynucleotide or polynucleotides are advantageously present in an inventive vector. In minimum manner, this comprises, consists essentially of, or consists of an initiation codon (ATG), a stop codon and a promoter, and optionally also a polyadenylation sequence for certain vectors such as plasmid and certain viral vectors, e.g., viral vectors other than poxviruses. When the polynucleotide encodes a polyprotein fragment, such as, but no limited to, any of the T. parva proteins Tp1-8, or fragments thereof, advantageously, in the vector, an ATG is placed at 5′ of the reading frame and a stop codon is placed at 3′. Other elements for controlling expression may be present, such as enhancer sequences, stabilizing sequences and signal sequences permitting the secretion of the protein.

A DNA “coding sequence” or a “nucleotide sequence encoding” a particular protein, is a DNA sequence which is transcribed and translated into a polypeptide in vitro or in vivo when placed under the control of appropriate regulatory elements. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence.

DNA “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated. A control element, such as a promoter, “directs the transcription” of a coding sequence in a cell when RNA polymerase will bind the promoter and transcribe the coding sequence into mRNA, which is then translated into the polypeptide encoded by the coding sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

A “host cell” is a cell that has been transformed, or is capable of transformation, by an exogenous nucleic acid molecule.

A cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the exogenous DNA.

The term “vaccine composition” as used herein refers to any pharmaceutical composition containing an antigen, which composition can be used to prevent or treat a disease or condition in a subject.

By “subunit vaccine composition” is meant a composition containing at least one immunogenic polypeptide or a polynucleotide able to express at least one, but not all, antigen derived from or homologous to an antigen from a pathogen of interest. Such a composition is substantially free of intact pathogen cells or particles, or the lysate of such cells or particles. Thus, a “subunit vaccine composition” is prepared from at least partially purified (preferably substantially purified) immunogenic polypeptides from the pathogen, or recombinant analogs thereof. A subunit vaccine composition can comprise the subunit antigen or antigens of interest substantially free of other antigens or polypeptides from the pathogen.

One aspect of the present invention encompasses recombinant vaccines against Theileria parva. The present invention therefore provides preparations comprising viral vectors such as viral expression vectors for use in vaccines or immunogenic compositions. The preparations can comprise, consist essentially of, or consist of one or more vectors, e.g., expression vectors, such as in vivo expression vectors, comprising, consisting essentially or consisting of (and advantageously expressing) one or more of the Theileria parva polynucleotides encoding such proteins as any of Tp1-8 (according to SEQ ID NOs. 9-16, or combinations or polyproteins thereof, such as, for example, of Tp1-8 or at least an epitope thereof, especially as described in PCT Application Serial No. PCT/US2004/022605 incorporated herein by reference in its entirety. Advantageously, the vector may contain and express a polynucleotide that includes, consists essentially of, or consists of a coding region encoding any of the proteins Tp1-8 or fragments thereof of Theileria parva, in a pharmaceutically or veterinarily acceptable carrier, excipient or vehicle. Thus, according to an embodiment of the invention, the other vector or vectors in the preparation comprises, consists essentially of or consists of a polynucleotide that encodes, and under appropriate circumstances the vector expresses one or more other proteins or regions thereof, of the Theileria parva, e.g. any of, but not limited to, Tp1-8.

In an advantageous embodiment, the expression vector is a viral vector. In a particularly advantageous embodiment, the viral vector is an avipox vector. In a more advantageous embodiment, the avipox vector is a canarypox vector or a fowlpox vector. More advantageously, the avipox vector is an attenuated avipox vector. In a particularly advantageous embodiment, the attenuated avipox vector is an attenuated canarypox or an attenuated fowlpox vector. Advantageously, the attenuated canarypox vector is ALVAC and the attenuated fowlpox vector is TROVAC.

The invention further encompasses polynucleotides encoding functionally equivalent variants and derivatives of Theileria parva polypeptides and functionally equivalent fragments thereof which may enhance, decrease or not significantly affect properties of the polypeptides encoded thereby. These functionally equivalent variants, derivatives, and fragments display the ability to retain the immunogenic activity of a Theileria parva polypeptide. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan.

Alternatively, homology can be determined by hybridization of polynucleotides under conditions that form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

Two nucleic acid fragments are considered to be “selectively hybridizable” to a polynucleotide if they are capable of specifically hybridizing to a nucleic acid or a variant thereof (e.g., a probe that hybridizes to a Theileria parva nucleic acid but not to polynucleotides from other genes or members of the Theileria genus) or specifically priming a polymerase chain reaction: (i) under typical hybridization and wash conditions, as described, for example, in Sambrook et al., supra and Nucleic Acid Hybridization, supra, (ii) using reduced stringency wash conditions that allow at most about 25-30% basepair mismatches, for example: 2×SSC, 0.1% SDS, room temperature twice, 30 minutes each; then 2×SSC, 0.1% SDS, 37° C. once, 30 minutes; then 2×SSC room temperature twice, 10 minutes each, or (iii) selecting primers for use in typical polymerase chain reactions (PCR) under standard conditions (described for example, in Saiki, et al. (1988) Science 239:487-491), which result in specific amplification of sequences of a Theileria parva geneor its variants.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

“Homology” refers to the percent identity between two polynucleotide or two polypeptide moieties. Two DNA, or two polypeptide sequences are “substantially homologous” to each other when the sequences exhibit at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

An example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, (1988) Proc. Natl. Acad. Sci. USA, 85: 2444-2448.

Advantageous for use according to the present invention is the WU-BLAST (Washington University BLAST) version 2.0 software. WU-BLAST version 2.0 executable programs for several UNIX platforms can be downloaded from ftp ://blast. wustl. edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology ” or “identity”, for instance, with respect to a nucleotide or amino acid sequence, can indicate a quantitative measure of identity between two sequences. The percent sequence identity can be calculated as (N_{ref}-N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

And, without undue experimentation, the skilled artisan can consult with many other programs or references for determining percent homology.

The term “capable of hybridizing under stringent conditions” as used herein refers to annealing a first nucleic acid to a second nucleic acid under stringent conditions as defined below. Stringent hybridization conditions typically permit the hybridization of nucleic acid molecules having at least 70% nucleic acid sequence identity with the nucleic acid molecule being used as a probe in the hybridization reaction. For example, the first nucleic acid may be a test sample or probe, and the second nucleic acid may be the sense or antisense strand of a nucleic acid encoding a Theileria parva polypeptide, or a fragment thereof. Hybridization of the first and second nucleic acids may be conducted under stringent conditions, e.g., high temperature and/or low salt content that tend to disfavor hybridization of dissimilar nucleotide sequences. Alternatively, hybridization of the first and second nucleic acid may be conducted under reduced stringency conditions, e.g. low temperature and/or high salt content that tend to favor hybridization of dissimilar nucleotide sequences. Low stringency hybridization conditions may be followed by high stringency conditions or intermediate medium stringency conditions to increase the selectivity of the binding of the first and second nucleic acids. The hybridization conditions may further include reagents such as, but not limited to, dimethyl sulfoxide (DMSO) or formamide to disfavor still further the hybridization of dissimilar nucleotide sequences. A suitable hybridization protocol may, for example, involve hybridization in 6×SSC (wherein 1×SSC comprises 0.015 M sodium citrate and 0.15 M sodium chloride), at 65° Celsius in an aqueous solution, followed by washing with 1×SSC at 65° C. Formulae to calculate appropriate hybridization and wash conditions to achieve hybridization permitting 30% or less mismatch between two nucleic acid molecules are disclosed, for example, in Meinkoth et al. (1984) Anal. Biochem. 138: 267-284; the content of which is herein incorporated by reference in its entirety. Protocols for hybridization techniques are well known to those of skill in the art and standard molecular biology manuals may be consulted to select a suitable hybridization protocol without undue experimentation. See, for example, Sambrook et al. (2001) Molecular Cloning: A Laboratory Manual, 3rd ed., Cold Spring Harbor Press, the contents of which are herein incorporated by reference in their entirety.

Typically, stringent conditions will be those in which the salt concentration is less than about 1.5 M sodium ion, typically about 0.01 to 1.0 M Na ion concentration (or other salts) from about pH 7.0 to about pH 8.3 and the temperature is at least about 30° Celsius for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° Celsius, and a wash in 1-2×SSC at 50 to 55° Celsius. Exemplary moderate stringency conditions include hybridization in 40 to 45% formamide, 1 M NaCl, 1% SDS at 37° Celsius, and a wash in 0.5-1×SSC at 55 to 60° Celsius. Exemplary high stringency conditions include hybridization in 50% formamide, 1 M NaCl, 1% SDS at 37° Celsius, and a wash in 0.1×SSC at 60 to 65° Celsius.

The invention further encompasses a Theileria parva protein, advantageously any of, but not limited to, polypeptides Tp1-8 or fragments thereof of Theileria parva and contained in a vector molecule or an expression vector and operably linked to an enhancer and/or a promoter element if necessary. In one embodiment, the promoter is a cytomegalovirus (CMV) promoter. In another embodiment, the enhancers and/or promoters include various cell or tissue specific promoters, various viral promoters and enhancers and various Theileria parva DNA sequences isogenically specific for each animal species. Advantageously, poxvirus promoters, such as but not limited to vaccinia and entomopox virus promoters, are used for poxvirus expression vectors.

A “vector” refers to a recombinant DNA or RNA plasmid or virus that comprises a heterologous polynucleotide to be delivered to a target cell, either in vitro or in vivo. The heterologous polynucleotide may comprise a sequence of interest for purposes of therapy, and may optionally be in the form of an expression cassette. As used herein, a vector need not to be capable of replication in the ultimate target cell or subject. The term includes cloning vectors for translation of a polynucleotide encoding sequence. Also included are viral vectors.

The term “recombinant” means a cloned DNA or cDNA fragment or a synthetic DNA fragment which either does not occur in nature or is linked to another polynucleotide in an arrangement not found in nature.

A “heterologous” region of a DNA construct is an identifiable segment of DNA within or attached to another DNA molecule that is not found in association with the other molecule in nature. Thus, when the heterologous region encodes a protozoal gene or fragment thereof, the gene will usually be flanked by DNA that does not flank the protozoal gene in the genome of the source virus. Another example of the heterologous coding sequence is a construct where the coding sequence itself is not found in nature (e.g., synthetic sequences having codons different from the native gene). Allelic variation or naturally occurring mutational events do not give rise to a heterologous region of DNA, as used herein.

The heterologous gene can be placed under the control of a promoter, ribosome binding site (for bacterial expression) and, optionally, an enhancer or operator (collectively referred to herein as “control” elements), so that the DNA sequence encoding the desired protein is transcribed into RNA in the host cell transformed by a vector containing this expression construction. The coding sequence may or may not contain a signal peptide or leader sequence. If a signal sequence is included, it can either be the native, homologous sequence, or a heterologous sequence. Leader sequences can be removed by the host in post-translational processing. See, e.g., U.S. Pat. Nos. 4,431,739; 4,425,437; 4,338,397.

Control elements and other regulatory sequences may be ligated to the coding sequence prior to insertion into a vector, such as the cloning vectors described above. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. It may also be desirable to produce mutants or analogs of the desired Theileria protein. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are described in, e.g., Sambrook et al., supra; DNA Cloning, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Mandin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insert hosts useful in the present invention include, but are not limited to, Spodoptera frugiperda cells.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6; 312,683; U.S. patent application Ser. No. 920,197, filed Oct. 16, 1986; WO 90/01543; W091/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312. Thus, the vector in the invention can be any suitable recombinant virus or virus vector, such as a poxvirus (e.g., vaccinia virus, avipox virus, canarypox virus, fowlpox virus, raccoonpox virus, swinepox virus, etc.), adenovirus (e.g., canine adenovirus), herpesvirus, baculovirus, retrovirus, etc. (as in documents incorporated herein by reference); or the vector can be a plasmid. The herein cited and incorporated herein by reference documents, in addition to providing examples of vectors useful in the practice of the invention, can also provide sources for non-Theileria parva proteins or fragments thereof, e.g., non-Theileria parva proteins or fragments thereof, cytokines, etc. to be expressed by vector or vectors in, or included in, the compositions of the invention.

The present invention also provides preparations comprising vectors, such as expression vectors, e.g., therapeutic compositions. The preparations can comprise, consist essentially of, or consist of one or more vectors, e.g., expression vectors, such as in vivo expression vectors, comprising, consisting essentially or consisting of (and advantageously expressing) one or more of Theileria parva polynucleotides and, advantageously, the vector contains and expresses a polynucleotide that includes, consists essentially of, or consists of a coding region encoding a Theileria parva protein in a pharmaceutically or veterinarily acceptable carrier, excipient or vehicle. Thus, according to an embodiment of the invention, the other vector or vectors in the preparation comprises, consists essentially of or consists of a polynucleotide that encodes, and under appropriate circumstances the vector expresses one or more other proteins of a Theileria parva, or a fragment thereof.

According to another embodiment, the vector or vectors in the preparation comprise, or consist essentially of, or consist of polynucleotide(s) encoding one or more proteins or fragment(s) thereof of a Theileria parva protein. The inventive preparation advantageously comprises, consists essentially of, or consists of, at least two vectors comprising, consisting essentially of, or consisting of, and advantageously also expressing, advantageously in vivo under appropriate conditions or suitable conditions or in a suitable host cell, polynucleotides from different Theileria parva isolates encoding the same proteins and/or for different proteins, but advantageously for the same proteins. As to preparations containing one or more vectors containing, consisting essentially of or consisting of polynucleotides encoding, and advantageously expressing, advantageously in vivo, a Theileria parva protein, or an epitope thereof, it is advantageous that the expression products be from two, three or more different Theileria parva isolates, advantageously strains. The invention is also directed at mixtures of vectors that contain, consist essentially of, or consist of coding for, and express, different Theileria parva proteins.

In an advantageous embodiment, the vector is a viral vector, advantageously an avipox vector containing a Theileria parva gene or a fragment thereof. In a particularly advantageous embodiment, the avipox vector is a canary pox vector, advantageously, an attenuated canarypox vector such as ALVAC. Attenuated canarypox viruses are described in U.S. Pat. No. 5,756,103 (ALVAC) and WO01/05934. In another particularly advantageous embodiment, the avipox vector is a fowlpox vector, advantageously an attenuated fowlpox vector such as TROVAC. Reference is also made to U.S. Pat. No. 5,766,599 that pertains to the attenuated fowlpox strain TROVAC. In this regard, reference is made to the canarypox available from the ATCC under access number VR-111. Numerous fowlpox virus vaccination strains are also available, e.g. the DIFTOSEC CT strain marketed by MERIAL and the NOBILIS VARIOLE vaccine marketed by INTERVET; and, reference is also made to U.S. Pat. No. 5,766,599 which pertains to the atenuated fowlpox strain TROVAC.

In one particular embodiment, the viral vector is a poxvirus, e.g. a vaccinia virus or an attenuated vaccinia virus, (for instance, MVA, a modified Ankara strain obtained after more than 570 passages of the Ankara vaccine strain on chicken embryo fibroblasts; see Stickl & Hochstein-Mintzel, Munch. Med. Wschr., 1971, 113, 1149-1153; Sutter et al., Proc. Natl. Acad. Sci. U.S.A., 1992, 89, 10847-10851; available as ATCC VR-1508; orNYVAC, see U.S. Pat. No. 5,494,807, for instance, Examples 1 to 6 and et seq of U.S. Pat. No. 5,494,807 which discuss the construction of NYVAC, as well as variations of NYVAC with additional ORFs deleted from the Copenhagen strain vaccinia virus genome, as well as the insertion of heterologous coding nucleic acid molecules into sites of this recombinant, and also, the use of matched promoters; see also WO96/40241), an avipox virus or an attenuated avipox virus (e.g., canarypox, fowlpox, dovepox, pigeonpox, quailpox, ALVAC or TROVAC; see, e.g., U.S. Pat. No. 5,505,941, 5,494,807), swinepox, raccoonpox, camelpox, or myxomatosis virus.

For information on the method to generate recombinants thereof and how to administer recombinants thereof, the skilled artisan can refer documents cited herein and to WO90/12882, e.g., as to vaccinia virus mention is made of U.S. Pat. Nos. 4,769,330, 4,722,848, 4,603,112, 5,110,587, 5,494,807, and 5,762,938 inter alia; as to fowlpox, mention is made of U.S. Pat. Nos. 5,174,993, 5,505,941 and U.S. Pat. No 5,766,599 inter alia; as to canarypox mentionis made of U.S. Pat. No. 5,756,103 inter alia; as to swinepox mention is made of U.S. Pat. No. 5,382,425 inter alia; and, as to raccoonpox, mention is made of WO00/03030 inter alia.

When the expression vector is a vaccinia virus, insertion site or sites for the polynucleotide or polynucleotides to be expressed are advantageously at the thymidine kinase (TK) gene or insertion site, the hemagglutinin (HA) gene or insertion site, the region encoding the inclusion body of the A type (ATI); see also documents cited herein, especially those pertaining to vaccinia virus. In the case of canarypox, advantageously the insertion site or sites are ORF(s) C3, C5 and/or C6; see also documents cited herein, especially those pertaining to canarypox virus. In the case of fowlpox, advantageously the insertion site or sites are ORFs F7 and/or F8; see also documents cited herein, especially those pertaining to fowlpox virus. The insertion site or sites for MVA virus are advantageously as in various publications, including Carroll M. W. et al., Vaccine, 1997, 15 (4), 387-394; Stittelaar K. J. et al., J. Virol., 2000, 74 (9), 4236-4243; Sutter G. et al., 1994, Vaccine, 12 (11), 1032-1040; and, in this regard it is also noted that the complete MVA genome is described in Antoine G., Virology, 1998, 244, 365-396, which enables the skilled artisan to use other insertion sites or other promoters.

Advantageously, the polynucleotide to be expressed is inserted under the control of a specific poxvirus promoter, e.g., the vaccinia promoter 7.5 kDa (Cochran et al., J. Virology, 1985, 54, 30-35), the vaccinia promoter I3L (Riviere et al., J. Virology, 1992, 66, 3424-3434), the vaccinia promoter HA (Shida, Virology, 1986, 150, 451-457), the cowpox promoter ATI (Funahashi et al., J. Gen. Virol., 1988, 69, 35-47), the vaccinia promoter H6 (Taylor J. et al., Vaccine, 1988, 6, 504-508; Guo P. et al. J. Virol., 1989, 63, 4189-4198; Perkus M. et al., J. Virol., 1989, 63, 3829-3836), inter alia.

Advantageously, for the vaccination of mammals the expression vector is a canarypox or a fowlpox. In this way, there can be expression of the heterologous proteins with limited or no productive replication.

The term plasmid covers any DNA transcription unit comprising a polynucleotide according to the invention and the elements necessary for its in vivo expression in a cell or cells of the desired host or target; and, in this regard, it is noted that a supercoiled or non-supercoiled, circular plasmid, as well as a linear form, are intended to be within the scope of the invention.

Plasmids according to the invention may comprise or contain or consist essentially of, in addition to the polynucleotide encoding a Theileria parva-derived polypeptide, variant, analog or fragment, operably linked to a promoter or under the control of a promoter or dependent upon a promoter. In general, it is advantageous to employ a strong promoter functional in eukaryotic cells. The preferred strong promoter is the immediate early cytomegalovirus promoter (CMV-IE) of human or murine origin, or optionally having another origin such as the rat or guinea bovine. The CMV-IE promoter can comprise the actual promoter part, which may or may not be associated with the enhancer part. Reference can be made to EP-A-260 148, EP-A-323 597, U.S. Pat. Nos. 5,168,062, 5,385,839, and 4,968,615, as well as to PCT Application No WO87/03905. The CMV-IE promoter is advantageously a human CMV-IE (Boshart M. et al., Cell, 1985, 41, 521-530) or murine CMV-IE.

In more general terms, the promoter has either a viral or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa M. et al., Vaccine, 2000, 18, 2337-2344), or the actin promoter (Miyazaki J. et al., Gene, 1989, 79, 269-277).

Functional sub fragments of promoters, i.e., portions of promoters that maintain an adequate promoting activity, are included within the present invention, e.g. truncated CMV-IE promoters according to PCT Application No. WO98/00166 or U.S. Pat. No. 6,156,567 can be used in the practice of the invention. A promoter in the practice of the invention consequently may include derivatives and sub fragments of a full-length promoter that maintain an adequate promoting activity and hence function as a promoter, preferably promoting activity substantially similar to that of the actual or full-length promoter from which the derivative or sub fragment is derived, e.g., akin to the activity of the truncated CMV-IE promoters of U.S. Pat. No. 6,156,567 to the activity of full-length CMV-IE promoters. Thus, a CMV-IE promoter in the practice of the invention can comprise or consist essentially of or consist of the promoter portion of the full-length promoter and/or the enhancer portion of the full-length promoter, as well as derivatives and sub fragments.

Advantageously, the vectors comprise or consist essentially of other expression control elements. It is particularly advantageous to incorporate stabilizing sequence(s), e.g., intron sequence(s), preferably the first intron of the hCMV-IE (PCT Application No. WO89/01036), the intron II of the rabbit β-globin gene (van Ooyen et al., Science, 1979, 206, 337-344).

As to the polyadenylation signal (polyA) for the plasmids and viral vectors other than poxviruses, use can be made of the poly(A) signal of the bovine growth hormone (bGH) gene (see U.S. Pat. No. 5,122,458), or the poly(A) signal of the rabbit β-globin gene or the poly(A) signal of the SV40 virus and the like.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

In the invention, preferably the polynucleotide also comprises a nucleotide sequence encoding a signal peptide, located upstream of the coding for the expressed protein to facilitate the secretion thereof; and accordingly, the invention comprehends the expression of a Theileria parva polypeptide, such as a Theileria parva antigen, immunogen, or fragment thereof, e.g., epitope, with a leader or signal sequence. The leader or signal sequence can be an endogenous sequence, e.g. the natural signal sequence of a Theileria parva polypeptide, which can be from the same Theileria parva strain or isolate or another strain or isolate. The leader or signal sequence can also be a heterologous sequence, and thus encoded by a nucleotide sequence that is heterologous to Theileria parva. For example, the leader or signal sequence can be endogenous to the vector, or a leader or signal sequence that is heterologous to both the vector and Theileria parva, such as a signal peptide of tissue plasminogen activator (tPA), e.g., human tPA, and thus, the vector or the polynucleotide therein can include a sequence encoding the leader or signal peptide, e.g., the leader or signal peptide of human tissue plasminogen activator (tPA) (Hartikka J. et al., Human Gene Therapy, 1996, 7, 1205-1217). The nucleotide sequence encoding the signal peptide is advantageously inserted in frame and upstream of the sequence encoding the Theileria parva polypeptide.

However, it is also noted that it can be advantageous that the vector not be a natural pathogen of the host; for instance, so that the vector can have expression of the exogenous, e.g., Theileria parva coding sequences, but with limited or no replication; for example, the use of an avipox vector in a mammalian host, as in U.S. Pat. No. 5,174,993. It is also noted that the invention comprehends vaccines, immunological and immunogenic compositions, with those terms being used in the sense attributed to them in the art; see, e.g., documents cited herein, such as U.S. Pat. No. 6,497,883.

It is understood to one of skill in the art that conditions for culturing a host cell varies according to the particular gene and that routine experimentation is necessary at times to determine the optimal conditions for culturing a Theileria parva protein, depending on the host cell. A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof.

Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

Protein production can take place by the transfection of mammalian cells by plasmids, by replication or expression without productive replication of viral vectors on mammal cells or avian cells, or by Baculovirus replication (see, e.g., U.S. Pat. No. 4,745,051; Vialard J. et al., J. Virol., 1990 64 (1), 37-50; Verne A., Virology, 1988, 167, 56-71), e.g. Autographa californica Nuclear Polyhedrosis Virus AcNPV, on insect cells (e.g. Sf9 Spodoptera frugiperda cells, ATCC CRL 1711; see also U.S. Pat. Nos. 6,228,846, 6,103,526). Mammalian cells that can be used are advantageously hamster cells (e.g. CHO or BHK-21) or-monkey cells (e.g. COS or VERO). Thus, the invention accordingly encompasses expression vectors incorporating a polynucleotide according to the invention, as well as the thus produced or expressed Theileria parva proteins or fragments thereof from in vitro expression, and the preparations containing the same.

Accordingly, the present invention also relates to Theileria parva protein-concentrated and/or purified preparations. When the polynucleotide encodes several proteins, they are cleaved, and the aforementioned preparations then contain cleaved proteins.

The present invention also relates to immunogenic compositions and vaccines against the Theileria parva comprising at least one in vivo expression vector according to the invention and a pharmaceutically or veterinarily acceptable excipient or carrier or vehicle, and optionally an adjuvant.

An immunogenic composition covers any composition that, once administered to the target species, induces an immune response against the Theileria parva. The term vaccine is understood to mean a composition able to induce an effective protection. The target species include mammals, e.g., bovines, porcines and humans.

In an advantageous embodiment, the invention provides for the administration of a therapeutically effective amount of a formulation for the delivery and expression of a Theileria parva protein, or an immunogenic fragment thereof in a target cell. Determination of the therapeutically effective amount is routine experimentation for one of ordinary skill in the art. In one embodiment, the formulation comprises an expression vector comprising a polynucleotide that expresses a Theileria parva protein, and a pharmaceutically or veterinarily acceptable carrier, vehicle or excipient. In an advantageous embodiment, the pharmaceutically or veterinarily acceptable carrier, vehicle or excipient facilitates transfection and/or improves preservation of the vector or protein.

The pharmaceutically or veterinarily acceptable carriers or vehicles or excipients are well known to the one skilled in the art. For example, a pharmaceutically or veterinarily acceptable carrier or vehicle or excipient can be a 0.9% NaCl (e.g., saline) solution or a phosphate buffer. Other pharmaceutically or veterinarily acceptable carrier or vehicle or excipients that can be used for methods of this invention include, but are not limited to, poly-(L-glutamate) or polyvinylpyrrolidone. The pharmaceutically or veterinarily acceptable carrier or vehicle or excipients may be any compound or combination of compounds facilitating the administration of the vector (or protein expressed from an inventive vector in vitro); advantageously, the carrier, vehicle or excipient may facilitate transfection and/or improve preservation of the vector (or protein). Doses and dose volumes are herein discussed in the general description and can also be determined by the skilled artisan from this disclosure read in conjunction with the knowledge in the art, without any undue experimentation.

Adjuvants that enhance the effectiveness of the vaccine may also be added to the formulation. Such adjuvants include, without limitation, adjuvants formed from aluminum salts (alum), such as aluminum hydroxide, aluminum phosphate, aluminum sulfate, etc; oil-in-water and water-in-oil emulsion formulations, such as Incomplete Freunds Adjuvant (IFA), avridine and dimethyldioctadecyl ammonium bromide (DDA); adjuvants formed from bacterial cell wall components such as adjuvants including monophosphoryl lipid A (MPL) (Imoto et al. (1985) Tet. Lett. 26:1545-1548), trehalose dimycolate (TDM), and cell wall skeleton (CWS); adjuvants derived from ADP-ribosylating bacterial toxins, such as derived from diphtheria toxin (for example, CRM,₁₉₇, a non-toxic diphtheria toxin mutant (see, e.g., Bixler et al. (1989) Adv. Exp. Med. Biol. 251:175; and Constantino et al. (1992) Vaccine), pertussis toxin (PT), cholera toxin (CT), the E. coli heat-labile toxins (LT1 and LT2), Pseudomonas endotoxin A, C. botulinum C2 and C3 toxins, as well as toxins from C. perfringens, C. spiriforma and C. difficile; saponin adjuvants such as Quil A (U.S. Pat. No. 5,057,540), or particles generated from saponins such as ISCOMs (immunostimulating complexes); cytokines, such as interleukins (e.g., IL-1, IL-2, IL-4, IL-5, IL-6, IL-7, IL-12, etc.), interferons (e.g., γ-interferon), macrophage colony stimulating factor (M-CSF), tumor necrosis factor (TNF), etc; muramyl peptides such as N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3 huydroxyphosphoryloxy)-ethylamine (MTP-PE), etc.; adjuvants derived from the CpG family of molecules, CpG dinucleotides and synthetic oligonucleotides which comprise CpG motifs (see, e.g., Krieg et al. Nature (1995) 374:546 and Davis et al. J. Immunol. (1998) 160:870-876); and synthetic adjuvants such as PCPP (Poly di(carboxylatophenoxy)phosphazene) (Payne et al. Vaccines (1998) 16:92-98). Such adjuvants are commercially available from a number of distributors such as Accurate Chemicals; Ribi Immunechemicals, Hamilton, Mont.; GIBCO; Sigma, St. Louis, Mo.

The oil in water emulsion, which is especially appropriate for viral vectors, can be based on: light liquid paraffin oil (European pharmacopoeia type), isoprenoid oil such as squalane, squalene, oil resulting from the oligomerization of alkenes, e.g. isobutene or decene, esters of acids or alcohols having a straight-chain alkyl group, such as vegetable oils, ethyl oleate, propylene glycol, di(caprylate/caprate), glycerol tri(caprylate/caprate) and propylene glycol dioleate, or esters of branched, fatty alcohols or acids, especially isostearic acid esters. The oil is used in combination with emulsifiers to form an emulsion. The emulsifiers may be nonionic surfactants, such as: esters of on the one hand sorbitan, mannide (e.g. anhydromannitol oleate), glycerol, polyglycerol or propylene glycol and on the other hand oleic, isostearic, ricinoleic or hydroxystearic acids, said esters being optionally ethoxylated, polyoxypropylene-polyoxyethylene copolymer blocks, such as Pluronic, e.g., L121.

As to the maleic anhydride-alkenyl derivative copolymers, preference is given to EMA (Monsanto), which are straight-chain or crosslinked ethylene-maleic anhydride copolymers and they are, for example, crosslinked by divinyl ether. Reference is also made to J. Fields et al., Nature 186: 778-780, Jun. 4, 1960. With regard to structure, the acrylic or methacrylic acid polymers and EMA are preferably formed by basic units having the following formula in which: R₁ and R₂, which can be the same or different, represent H or CH₃,x=0 or 1, preferably x=1, y=1 or 2, with x+y=2. For EMA, x=0 and y=2 and for carbomers x=y=1. These polymers are soluble in water or physiological salt solution (20 g/l NaCl) and the pH can be adjusted to 7.3 to 7.4, e.g., by soda (NaOH), to provide the adjuvant solution in which the expression vector(s) can be incorporated. The polymer concentration in the final vaccine composition can range between 0.01 and 1.5% w/v, advantageously 0.05 to 1% w/v and preferably 0.1 to 0.4% w/v.

It is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book. For example the adjuvant-containing vaccine is prepared in the following way: 67% v/v of aqueous phase comprising the immunogen are emulsified in 2,3% w/v of anhydromannitol oleate, 2,6% w/v of oleic acid ethoxylated with 11 EO (ethylene oxide) and 28,1% v/v of light liquid paraffin oil (European Pharmacopea type) with the aid of an emulsifying turbomixer.

An alternative method for preparing the emulsion consists in emulsifying, by passages through a high-pressure homogenizer, a mixture of 5% w/v squalane, 2.5% w/v Pluronic® L121, 0.2% w/v of an ester of oleic acid and of anhydrosorbitol ethoxylated with 20 EO, 92.3% v/v of the aqueous phase comprising the immunogen.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 (incorporated herein by reference) which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol® (BF Goodrich, Ohio, USA) are particularly appropriate. They are cross-linked with an allyl sucrose or with allyl pentaerythritol. Among then, there may be mentioned Carbopol® 974P, 934P and 971P. Among the copolymers of maleic anhydride and alkenyl derivative, the copolymers EMA® (Monsanto) which are copolymers of maleic anhydride and ethylene, linear or cross-linked, for example cross-linked with divinyl ether, are preferred. Reference may be made to J. Fields et al., Nature, 186 : 778-780, Jun. 4, 1960, incorporated herein by reference.

The present invention also relates to immunogenic compositions and so-called subunit vaccines, incorporating or comprising or consisting essentially of one or more Tp1-Tp8 proteins of Theileria parva as described in PCT application Serial No. PCT/US2004/022605 incorporated herein by reference in its entirety and advantageously produced by in vitro expression in the manner described herein, as well as a pharmaceutically or veterinarily acceptable carrier or vehicle or excipient. Other proteins of Theileria parva can also be added.

The pharmaceutically or veterinarily acceptable carriers or vehicles or excipients are well known to the one skilled in the art. For example, a pharmaceutically or veterinarily acceptable carrier or vehicle or excipient can be a 0.9% NaCl (e.g., saline) solution or a phosphate buffer. Other pharmaceutically or veterinarily acceptable carrier or vehicle or excipients that can be used for methods of this invention include, but are not limited to, poly-(L-glutamate) or polyvinylpyrrolidone. The pharmaceutically or veterinarily acceptable carrier or vehicle or excipients may be any compound or combination of compounds facilitating the administration of the vector (or protein expressed from an inventive vector in vitro); advantageously, the carrier, vehicle or excipient may facilitate transfection and/or improve preservation of the vector (or protein). Doses and dose volumes are herein discussed in the general description and can also be determined by the skilled artisan from this disclosure read in conjunction with the knowledge in the art, without any undue experimentation.

The cationic lipids containing a quaternary ammonium salt which are advantageously but not exclusively suitable for plasmids, are advantageously those having the following formula: 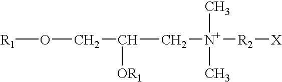
in which R₁ is a saturated or unsaturated straight-chain aliphatic radical having 12 to 18 carbon atoms, R₂ is another aliphatic radical containing 2 or 3 carbon atoms and X is an amine or hydroxyl group, e.g. the DMRIE. In another embodiment the cationic lipid can be associated with a neutral lipid, e.g. the DOPE.

Among these cationic lipids, preference is given to DMRIE (N-(2-hydroxyethyl)-N,N-dimethyl-2,3-bis(tetradecyloxy)-1-propane ammonium; WO96/34109), advantageously associated with a neutral lipid, advantageously DOPE (dioleoyl-phosphatidyl-ethanol amine; Behr J. P., 1994, Bioconjugate Chemistry, 5, 382-389), to form DMRIE-DOPE.

Advantageously, the plasmid mixture with the adjuvant is formed extemporaneously and advantageously contemporaneously with administration of the preparation or shortly before administration of the preparation; for instance, shortly before or prior to administration, the plasmid-adjuvant mixture is formed, advantageously so as to give enough time prior to administration for the mixture to form a complex, e.g. between about 10 and about 60 minutes prior to administration, such as approximately 30 minutes prior to administration.

When DOPE is present, the DMRIE:DOPE molar ratio is advantageously about 95: about 5 to about 5 :about 95, more advantageously about 1: about 1, e.g., 1:1.

The DMRIE or DMRIE-DOPE adjuvant:plasmid weight ratio can be between about 50: about 1 and about 1: about 10, such as about 10: about 1 and about 1:about 5, and advantageously about 1: about 1 and about 1: about 2, e.g., 1:1 and 1:2.

In a specific embodiment, the pharmaceutical composition is directly administered in vivo, and the encoded product is expressed by the vector in the host. The methods of in vivo delivery of a vector encoding a Theileria parva peptide (see, e.g., U.S. Pat. No. 6,423,693; patent publications EP 1052286, EP 1205551, U.S. patent publication 20040057941, WO 9905300 and Draghia-Akli et al., Mol Ther. (2002);6: 830-6; the disclosures of which are incorporated by reference in their entireties) can be modified to deliver the peptides of the present invention to a bovine. The in vivo delivery of a vector encoding a Theileria parva peptide described herein can be accomplished by one of ordinary skill in the art given the teachings of the above-mentioned references.

Advantageously, the pharmaceutical and/or therapeutic compositions and/or formulations according to the invention comprise or consist essentially of or consist of an effective quantity to elicit a therapeutic response of one or more expression vectors and/or polypeptides as discussed herein; and, an effective quantity can be determined from this disclosure, including the documents incorporated herein, and the knowledge in the art, without undue experimentation.

One skilled in the art can determine the effective plasmid dose to be used for each immunization or vaccination protocol and species from this disclosure and the knowledge in the art.

These methods can comprise, consist essentially of or consist of the administration of an effective quantity of an immunogenic composition or vaccine according to the invention. This administration can be by the parenteral route, e.g. by subcutaneous, intradermic (e.g., by ear) or intramuscular administration, and/or by oral and/or nasal routes. Advantageously, this administration is intramuscularly or subcutaneously. One or more administrations can take place, such as two administrations.

A needlefree liquid jet injector or powder jet injector can inject vaccines or immunogenic compositions. For plasmids it is also possible to use gold particles coated with plasmid and ejected in such a way as to penetrate the cells of the skin of the subject to be immunized (Tang et al., Nature 1992, 356, 152-154). Other documents cited and incorporated herein may be consulted for administration methods and apparatus of vaccines or immunogenic compositions of the invention. The needlefree injector can also be for example Biojector 2000 (Bioject Inc., Portland Oreg. USA).

Advantageously, the immunogenic compositions and vaccines according to the invention comprise or consist essentially of or consist of an effective quantity to elicit an immunological response and/or a protective immunological response of one or more expression vectors and/or polypeptides as discussed herein; and, an effective quantity can be determined from this disclosure, including the documents incorporated herein, and the knowledge in the art, without undue experimentation.

In the case of therapeutic and/or pharmaceutical compositions based on a plasmid vector, a dose can comprise, consist essentially of or consist of, in general terms, about in 1 μg to about 2000 μg, advantageously about 50 μg to about 1000 μg and more advantageously from about 100 μg to about 800 μg of plasmid expressing a Theileria parva peptide. When the therapeutic and/or pharmaceutical compositions based on a plasmid vector is administered with electroporation the dose of plasmid is generally between about 0.1 μg and 1 mg, advantageously between about 1 μg and 100 μg, advantageously between about 2 μg and 50 μg. The dose volumes can be between about 0.1 and about 2 ml, advantageously between about 0.2 and about 1 ml. These doses and dose volumes are suitable for the treatment of bovines and other mammalian target species.

The therapeutic and/or pharmaceutical composition contains per dose from about 10⁴ to about 10¹¹, advantageously from about 10⁵ to about 10¹⁰ and more advantageously from about 10⁶ to about 10⁹ viral particles of recombinant adenovirus expressing a Theileria parva peptide. In the case of therapeutic and/or pharmaceutical compositions based on a poxvirus, a dose can be between about 10² pfu and about 10⁹ pfu. The pharmaceutical composition contains per dose from about 10⁵ to 10⁹, advantageously from about 10⁶ to 10⁸ pfu of poxvirus or recombinant expressing a Theileria parva peptide.

The dose volume of immunogenic and vaccine compositions for target species such as bovines, e.g., the dose volume of equine immunogenic or vaccine compositions, based on viral vectors, e.g., non-poxvirus-viral-vector-based immunogenic or vaccine compositions, is generally between about 0.5 and about 2.5 ml, such as between about 0.5 and about 2.0 ml, preferably between about 1.0 and about 2.0 ml, preferably about 1.0 ml. Also in connection with such a vaccine or immunogenic composition, from the disclosure herein and the knowledge in 30 the art, the skilled artisan can determine the number of administrations, the administration route, and the doses to be used for each immunization or vaccination protocol, without any undue experimentation. For instance, there can be two administrations, e.g. at 35 day intervals.

In the case of subunit immunogenic compositions or subunit vaccines, with reference to the amount of active ingredient, e.g., subunit (antigen, immunogen, epitope) a dose comprises or consists essentially of or consists of, in general terms, about 10 μg to about 2000 μg, advantageously about 50 μg to approximately 1000 μg. The dose volume of such immunogenic or vaccine compositions for target species that are mammals, e.g., for equines, is generally between about 1.0 and about 2.0 ml, preferably between about 0.5 and about 2.0 ml and more advantageously about 1.0 ml. Also for such a vaccine or immunogenic composition, the skilled artisan, from this disclosure and the knowledge in the art, can, without any undue experimentation, determine the number of administrations, the administration route and the doses to be used for each immunization or vaccination protocol.

In an advantageous embodiment, the pharmaceutical and/or therapeutic compositions and/or formulations according to the invention are administered by injection, such as, but not limited to, intramuscular (IM), subcutaneous (SC) injection or intradermal (ID) injection. The injection may also be by needlefree delivery.

Also in connection with such a therapeutic composition, from the disclosure herein and the knowledge in the art, the skilled artisan can determine the number of administrations, the administration route, and the doses to be used for each injection protocol, without any undue experimentation.

In an advantageous embodiment, the recombinant vaccine can be administered to a bovine or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu, per dose, for example, per 2 ml dose. In a particularly advantageous embodiment, the dose is about 10⁸ pfu per dose.

The method includes at least one administration to an animal of an efficient amount of the therapeutic composition according to the invention. The animal may be male, female, pregnant female and newborn. This administration may be notably done by intramuscular (IM), intradermal (ID) or subcutaneous (SC) injection. In an advantageous embodiment, the therapeutic composition according to the invention can be administered by a syringe or a needleless apparatus (such as, for example Bovinejet, Biojector or Vitajet (Bioject, Oregon, USA)). Another approach to administer plasmid is to use electroporation see, e.g. S. Tollefsen et al. Vaccine, 2002, 20, 3370-3378; S. Tollefsen et al. Scand. J. Immunol., 2003, 57, 229-238; S. Babiuk et al., Vaccine, 2002, 20, 3399-3408; PCT Application No. WO99/01158.

The invention relates to the use of the pharmaceutical compositions for vaccinating in animals against Theileria parva infection. In an advantageous embodiment, the animal is a bovine.

Animals immunized with immunogenic compositions or vaccines according to the invention develop a specific immunity against Theileria parva, which during a Theileria parva infection involves a decrease of the blood-borne protozoa, and indeed can totally block the protozoa, as compared with unvaccinated control animals. This advantageous aspect of the invention may be used to stop the transmission of the Theileria parva, to limit the existence of protozoal reservoirs and to prevent outbreaks of East coast fever, notably in bovines.

Another advantageous aspect of the invention is that protective immunity can be transmitted from vaccinated subjects to the offspring.

According to the invention, the vaccination against the Theileria parva can be combined with other vaccinations within the framework of vaccination programs, in the form of immunization or vaccination kits or methods, or in the form of multivalent immunogenic compositions and multivalent vaccines, i.e. comprising or consisting essentially of at least one vaccine component against the Theileria parva and at least one vaccine component against at least one other pathogenic agent. This also includes the expression by the same expression vector of genes of at least two pathogenic agents, including the Theileria parva.

The invention thus also relates to a multivalent or “cocktail” immunogenic composition or a multivalent or “cocktail” vaccine against the Theileria parva and against at least one other pathogen of the target species, using the same in vivo expression vector containing and expressing at least one polynucleotide of the Theileria parva according to the invention and at least one polynucleotide expressing an immunogen of another pathogen. As to combination or multivalent or “cocktail” immunogenic compositions or vaccines, as well as to immunogens or antigens or epitopes thereof to be in or expressed by such compositions or vaccines, attention is directed to herein cited and incorporated by reference documents, as well as to U.S. Pat. Nos. 5,843,456 and 6,368,603.

The “immunogen” expressed by a vector of the invention or used in multivalent or “cocktail” compositions or vaccines is understood to mean a protein, glycoprotein, polypeptide, peptide, epitope or derivative, e.g. fusion protein, inducing an immune response, preferably of a protective nature.

As discussed herein, these multivalent compositions or vaccines can also comprise or consist essentially of a pharmaceutically or veterinarily acceptable carrier or vehicle or excipient, and optionally an adjuvant.

The invention also relates to a multivalent immunogenic composition or a multivalent vaccine comprising at least one in vivo expression vector in which at least one polynucleotide of the Theileria parva is inserted (and expressed in vivo) and at least a second expression vector in which a polynucleotide encoding an immunogen of another pathogenic agent is inserted (and expressed in vivo). Such multivalent compositions or vaccines also comprise or consist essentially of a pharmaceutically or veterinarily acceptable carrier or vehicle or excipient, and optionally an adjuvant.

For antigen(s) or immunogen(s) or epitope(s) to be included in or expressed by a multivalent immunogenic composition or vaccine (in addition to Theileria parva antigen(s), immunogen(s) or epitope(s)), including as to determining or ascertaining epitope(s), the skilled artisan may consult herein cited documents and documents cited in herein cited documents, all of which are incorporated by reference into the instant application.

The immunogenic compositions or vaccines as discussed herein can also be combined with at least one conventional vaccine (e.g., inactivated, live attenuated, or subunit) directed against the same pathogen or at least one other pathogen of the species to which the composition or vaccine is directed. The immunogenic compositions or vaccines discussed herein can be administered prior to or after the conventional vaccine, e.g., in a “prime-boost” regimen.

The invention further comprehends combined vaccination employing immunogenic composition(s) and subunit vaccine(s) according to the invention. Thus, the invention also relates to multivalent immunogenic compositions and multivalent vaccines comprising one or more proteins according to the invention and one or more immunogens (as the term immunogen is discussed herein) of at least one other pathogenic agent (advantageously from among those herein and in documents cited and incorporated herein by reference) and/or another pathogenic agent in inactivated or attenuated form or as a subunit. In the manner described, these multivalent vaccines or compositions also contain, consist essentially of or consist of a pharmaceutically or veterinarily acceptable vehicle or excipient and optionally one or more adjuvants.

The present invention also relates to methods for the immunization and vaccination of a target species, e.g., as discussed herein.

The present invention also relates to methods for the immunization and/or vaccination of a target species, using a prime-boost regimen. The term of “prime-boost” refers to the successive administrations of two different vaccine types or immunogenic or immunological composition types having at least one immunogen in common. The priming administration (priming) is the administration of a first vaccine or immunogenic or immunological composition type and may comprise one, two or more administrations. The boost administration is the administration of a second vaccine or immunogenic or immunological composition type and may comprise one, two or more administrations, and, for instance, may comprise or consist essentially of annual administrations.

An embodiment of a prime-boost immunization or vaccination against Theileria parva according to the invention is a prime-boost immunization or vaccination wherein the animal is first administered a (priming) DNA vaccine or immunological or immunogenic composition comprising or consisting essentially of and expressing in vivo at least one immunogen, antigen or epitope of Theileria parva, and thereafter is administered (boosted with) a second type of vaccine or immunogenic or immunological composition containing or consisting essentially of or expressing at least one immunogen, antigen or epitope that is common to the priming vaccine or immunogenic or immunological composition. This second type of vaccine can be a an inactivated, or attenuated or subunit vaccine or immunogenic or immunological composition or a vector, e.g., recombinant or modified virus vaccine or immunogenic or immunological composition that has in vivo expression (e.g. poxvirus, herpesvirus, adenovirus). Poxviruses may be advantageously employed, e.g., attenuated vaccinia viruses, like NYVAC or MVA, and avipox viruses, like canarypox viruses and fowlpox viruses.

Advantageously, the DNA vaccine is intended to induce a priming immune response specific for the expressed immunogen, antigen or epitope or “DNA induced immune response” (such as a gamma-interferon+(IFNγ⁺) T cell memory response specific for the expressed immunogen, antigen or epitope) which is boostable (can be boosted) by a subsequent administration (boost) of an inactivated vaccine or immunological composition or a live recombinant vaccine comprising or consisting essentially of a viral vector, such as a live recombinant poxvirus, containing or consisting essentially of and expressing in vivo at least the same immunogen(s) or antigen(s) or epitope(s) expressed by the DNA vaccine. The IFNγ⁺ T cell memory response specific for the expressed Theileria parva immunogen can be shown in a quantitative enzyme-linked immune spot (ELISPOT) assay using peripheral blood mononuclear cells (PBMCs) (Laval et al., Vet. Immunol. Immunopathol., 2002, 90: 191-201).

The “boost” may be administered from about 2 weeks to about 6 months after the “priming”, such as from about 3 to about 8 weeks after the priming, and advantageously from about 3 to about 6 weeks after the priming, and more advantageously, about 4 weeks after the priming.

The invention also relates to kits for performing prime-boost methods comprising or consisting essentially of a priming vaccine or immunological or immunogenic composition and a boost vaccine or immunological or immunogenic compositions in separate containers, optionally with instructions for admixture and/or administration.

The amounts (doses) administered in the priming and the boost and the route of administration for the priming and boost can be as herein discussed, such that from this disclosure and the knowledge in the art, the prime-boost regimen can be practiced without undue experimentation. Furthermore, from the disclosure herein and the knowledge in the art, the skilled artisan can practice the methods, kits, etc. herein with respect to any of the herein-mentioned target species.

One aspect of the present invention, therefore, is an avipox expression vector comprising a polynucleotide that encodes a Theileria parva polypeptide. In one embodiment of this aspect of the invention, the Theileria parva polypeptide may be selected from the group consisting of Tp1, Tp2, Tp3, Tp4, Tp5, Tp6, Tp7 and Tp8. In various embodiments of the invention, the Theileria parva polypeptide comprises an amino acid sequence according to any of the group consisting of SEQ ID NOs: 7, 16, 26, 30, 32, 39, 44-50, or a fragment thereof.

In other embodiments of the invention, the polynucleotide comprises a nucleotide base sequence according to any of the group consisting of SEQ ID NOs: 1, 6, 12, 15, 17, 19, 24, 25, 29, 33, 36, 38, 43, 51-60, or the complement thereof.

In yet another embodiment of the invention, the polynucleotide comprises a nucleotide sequence of the Theileria parva strain Muguga whole genome shotgun sequencing project, any fragment thereof or any complement thereof (see, e.g., Gardner et al., Science. 2005 Jul. 1; 309(5731):134-137, the disclosure of which is incorporated by reference) wherein SEQ ID NO: 69 corresponds to GenBank Accession No. AAGK01000001, Theileria parva polypeptide strain Muguga chromosome 1 chr1_complete, whole genome shotgun sequence; SEQ ID NO: 70 corresponds to GenBank Accession No. AAGK01000002, Theileria parva polypeptide strain Muguga chromosome 2 chr2_complete, whole genome shotgun sequence; SEQ ID NO: 71 corresponds to GenBank Accession No. AAGK01000003, Theileria parva polypeptide strain Muguga chromosome 4 ctg_{—}528, whole genome shotgun sequence; SEQ ID NO: 72 corresponds to GenBank Accession No. AAGK01000004, Theileria parva polypeptide strain Muguga chromosome 4 ctg_{—}529, whole genome shotgun sequence; SEQ ID NO: 73 corresponds to GenBank Accession No. AAGK01000005, Theileria parva polypeptide strain Muguga chromosome 3 ctg_{—}530, whole genome shotgun sequence; SEQ ID NO: 74 corresponds to GenBank Accession No. AAGK01000006, Theileria parva polypeptide strain Muguga chromosome 3 ctg_{—}531, whole genome shotgun sequence; SEQ ID NO: 75 corresponds to GenBank Accession No. AAGK01000007, Theileria parva polypeptide strain Muguga chromosome 3 ctg_{—}545, whole genome shotgun sequence; SEQ ID NO: 76 corresponds to GenBank Accession No. AAGK01000008, Theileria parva polypeptide strain Muguga chromosome 3 ctg_{—}546, whole genome shotgun sequence and SEQ ID NO: 77 corresponds to GenBank Accession No. AAGK01000009, Theileria parva polypeptide strain Muguga apicoplast, complete genome, whole genome shotgun sequence. In an advantageous embodiment, the polynucleotide encodes a Theileria parva polypeptide.

In the various embodiments of the invention, the avipox expression vector is an attenuated avipox expression vector. In one embodiment, the avipox expression vector is a canarypox vector. In an advantageous embodiment the attenuated canarypox vector is ALVAC.

In yet other embodiments of this aspect of the invention, the avipox expression vector is a fowlpox vector. In one embodiment, the attenuated fowlpox vector is TROVAC.

Another aspect of the invention is a formulation for delivery and expression of a Theileria parva polypeptide, wherein the formulation comprises an avipox expression vector comprising a polynucleotide that encodes a Theileria parva immunogen, and a pharmaceutically or veterinarily acceptable carrier, vehicle or excipient.

In one embodiment of the invention, the formulation may comprise an attenuated vaccinia virus comprising a polynucleotide having a nucleotide base sequence capable of hybridizing under stringent conditions to a nucleotide base sequence selected from the group consisting of SEQ ID NOs: 1, 6, 12, 15, 17, 19, 24, 25, 29, 33, 36, 38, 43, 51-60, or the complement thereof, and wherein the stringent hybridizing conditions are selected from (a) 40 to 45% formamide, 1 M NaCl, 1% SDS at 37° Celsius, and a wash in 0.5-1×SSC at 55 to 60° Celsius and (b) 50% formamide, 1 M NaCl, 1% SDS at 37° Celsius, and a wash in 0.1×SSC at 60 to 65° Celsius, and wherein the nucleotide base sequence has between about 75% and about 100%, between about 80% and about 100%, between about 85% and about 100%, between about 90% and about 100%, between about 95% and about 100%, between about 75% and about 90%, between about 80% and about 90%, between about 85% and about 100%, or between about 85% and about 95%, between about 85% and about 90% homology with a nucleotide sequence seletected from the group consisting of SEQ ID NOs: 1, 6, 12, 15, 17, 19, 24, 25, 29, 33, 36, 38, 43, 51-60, or the complement thereof.

In this aspect of the invention, one embodiment further comprises an attenuated vaccinia virus comprising a polynucleotide comprises a nucleotide base sequence according to any of the group consisting of SEQ ID NOs: 1, 6, 12, 15, 17, 19, 24, 25, 29, 33, 36, 38, 43, 51-60, or the complement thereof, or a fragment thereof. In another embodiment, the attenuated vaccinia virus is NYVAC.

Yet another aspect of the invention is a formulation for delivery and expression of a Theileria parva polypeptide, wherein the formulation comprises an avipox expression vector comprising a nucleotide base sequence according to any of the group consisting of SEQ ID NOs: 1, 6, 12, 15, 17, 19, 24, 25, 29, 33, 36, 38, 43, 51-60, or the complement thereof, or a fragment thereof, and wherein the avipox expression vector is an attenuated avipox expression vector, a canarypox vector, a fowlpox vector or an attenuated vaccinia virus. In one embodiment of this aspect, the fowlpox vector is TROVAC. In still another embodiment, the canarypox vector is an attenuated canarypox vector, advantageously ALVAC. In yet another embodiment, the attenuated vaccinia virus is NYVAC.

Another aspect of the invention is a method of delivering a Theileria parva antigen to an animal, comprising administering to the animal a formulation for delivery and expression of a Theileria parva immunogen, wherein the formulation comprises an avipox expression vector comprising a nucleotide base sequence according to any of the group consisting of SEQ ID NOs: 1, 6, 12, 15, 17, 19, 24, 25, 29, 33, 36, 38, 43, 51-60, or the complement thereof, and wherein the avipox expression vector is an attenuated avipox expression vector, a canarypox vector, a fowlpox vector or an attenuated vaccinia virus.

One embodiment of the invention is a method of eliciting an immune response against a strain of Theileria in an animal, comprising administering a formulation for delivery and expression of a Theileria parva polypeptide, wherein the formulation comprises an avipox expression vector comprising a nucleotide base sequence according to any of the group consisting of SEQ ID NOs: 1, 6, 12, 15, 17, 19, 24, 25, 29, 33, 36, 38, 43, 51-60, or the complement thereof, and wherein the avipox expression vector is an attenuated avipox expression vector, a canarypox vector, an attenuated canarypox vector, a fowlpox vector or an attenuated vaccinia virus and in an effective amount for eliciting an immune response.

Still another embodiment of the invention is a method of inducing an immunological or protective response against a strain of Theileria in an animal, comprising administering to the animal an effective amount of a formulation for delivery and expression of a Theileria parva immunogen, wherein the formulation comprises an avipox expression vector comprising a polynucleotide that encodes a Theileria parva immunogen and a pharmaceutically or veterinarily acceptable carrier, vehicle or excipient.

A method of eliciting an immune response against a strain of Theileria in an animal, comprising administering a composition comprising a cell, wherein the cell comprises an avipox expression vector comprising a polynucleotide that encodes a Theileria parva immunogen.

Another aspect of the invention is a kit for performing a method of inducing an immunological or protective response against a strain of Theileria in an animal comprising an avipox expression vector comprising a nucleotide base sequence that encodes a Theileria parva immunogen, or the complement thereof, and wherein the avipox expression vector is an attenuated avipox expression vector, a canarypox vector, an attenuated canarypox vector, a fowlpox vector or an attenuated vaccinia virus, and instructions for performing the method of delivery and expression of the vector in the animal.

Another embodiment of the invention is a kit for performing a method of inducing an immunological or protective response against a strain of Theileria in an animal comprising an avipox expression vector comprising a nucleotide base sequence according to any of the group consisting of SEQ ID NOs: 1, 6, 12, 15, 17, 19, 24, 25, 29, 33, 36, 38, 43, 51-60, or the complement thereof, and wherein the avipox expression vector is an attenuated avipox expression vector, a canarypox vector, a fowlpox vector or an attenuated vaccinia viand and instructions for performing the method of delivery and expression of the vector in an effective amount for eliciting an immune response in the animal.

It should be understood that the present invention is not limited to the specific compositions or methods described herein and that any composition having a formula or method steps equivalent to those described falls within the scope of the present invention. Preparation routes of the composition and method steps are merely exemplary so as to enable one of ordinary skill in the art to make the composition and use it according to the described process and its equivalents. It will also be understood that although the form of the invention shown and described herein constitutes advantageous embodiments of the invention, it is not intended to illustrate all possible forms of the invention. The words are words of description rather than of limitation. Various changes and variations may be made to the present invention without departing from spirit and scope of the invention.

The invention is further illustrated by the following non-limiting examples:

### EXAMPLE 1

### Generation of Targeted Gene List

Preliminary contigs of the T. parva genome were loaded into an annotation database at TIGR and subjected to an automated process that searched the T. parva contigs against a non-redundant database of proteins extracted from GenBank. The search results were used to produce a set of putative T. parva genes that encoded proteins similar to those in other organisms. This set of gene models was used to train the gene finding programs GlimmerM (Salzberg et al., (1999) Genomics 59: 24) and phat (Cawley et al., (2001) Biochem. Parasitol. 118: 167-), which were subsequently run against all of the preliminary contigs to produce gene models for the entire preliminary genome sequence. The proteins encoded by the predicted T. parva genes on chromosome 1 were subjected to database searches (blastp), predictions of signal peptides and signal anchors (SignalP-2.0 database) and transmembrane domains (TMHMM database). Fifty-five genes (Table 1) encoding candidate antigens were selected for cloning and her screening.

TABLE 1
Targeted gene list.

| Gene | Locus | Size | |
|---|---|---|---|
| ID | TP01- | (kb) | Protein |
| 1 | 0705 | 1.0 | hypothetical protein, |
| | | | conserved |
| 2 | 0200 | 0.4 | 40S ribosomal |
| | | | protein S16, putative |
| 3 | 0727 | 1.4 | glutamyl-tRNA |
| | | | synthetase, putative |
| 4 | 0424 | 0.5 | hypothetical protein |
| 5 | 0056 | 0.5 | hypothetical protein |
| 6 | 0493 | 0.7 | hypothetical protein |
| 7 | 1144 | 0.7 | hypothetical protein |
| 8 | 1145 | 0.7 | hypothetical protein |
| 9 | 0379 | 0.7 | hypothetical protein |
| 10 | 0378 | 0.7 | hypothetical protein |
| 11 | 0609 | 0.8 | Tash1 protein, |
| | | | putative |
| 12 | 0868 | 0.8 | hypothetical protein |
| 13 | 0095 | 0.8 | hypothetical protein |
| 14 | 0605 | 0.8 | Tash1 protein, |
| | | | putative |
| 15 | 0035 | 1.1 | DNA J protein, |
| | | | putative |
| 16 | 1013 | 1.1 | MtN3/RAG1IP |
| | | | protein, putative |
| 17 | 0603 | 1.0 | TashAT2 protein, |
| | | | putative |
| 18 | 0616 | 0.9 | Tash1 protein, |
| | | | putative |
| 19 | 0188 | 0.8 | prohibitin, putative |
| 20 | 1011 | 1.1 | hypothetical protein |
| 21 | 0719 | 1.1 | hypothetical protein |
| 22 | 1220 | 1.0 | hypothetical protein |
| 23 | 0914 | 1.1 | hypothetical protein |
| 24 | 0476 | 1.1 | aspartate |
| | | | carbamoyltransferase, |
| | | | putative |
| 25 | 0334 | 1.2 | hypothetical protein |
| 26 | 0967 | 1.6 | hypothetical protein |
| 27 | 0613 | 1.3 | Tash1 protein, |
| | | | putative |
| 28 | 0604 | 1.4 | TashAT3 protein, |
| | | | putative |
| 29 | 1069 | 1.4 | monosaccharide |
| | | | transporter, |
| | | | putative |
| 30 | 0016 | 2.7 | sortilin, putative |
| 31 | 0002 | 1.4 | hypothetical |
| | | | telomeric SfiI |
| | | | fragment 20 |
| | | | protein 2 |
| 32 | 0960 | 1.4 | hypothetical |
| | | | protein |
| 33 | 0615 | 1.4 | Tash1 protein, |
| | | | putative |
| 34 | 0715 | 1.4 | hypothetical |
| | | | protein |
| 35 | 0006 | 1.5 | hypothetical |
| | | | protein |
| 36 | 0487 | 1.4 | hypothetical |
| | | | protein |
| 37 | 0862 | 1.6 | GMP synthase, |
| 38 | 0401 | 1.5 | putative |
| | | | hypothetical |
| | | | protein, |
| | | | conserved |
| 39 | 1091 | 1.7 | hypothetical |
| | | | protein |
| 40 | 0701 | 1.8 | hypothetical |
| | | | protein |
| 41 | 0274 | 1.8 | hypothetical |
| | | | protein |
| 42 | 0289 | 3.0 | hypothetical |
| | | | protein |
| 43 | 0700 | 2.1 | hypothetical |
| | | | protein |
| 44 | 0509 | 1.9 | hypothetical |
| | | | protein |
| 45 | 1185 | 2.0 | hypothetical |
| | | | protein |
| 46 | 0031 | 2.0 | Glutamyl-tRNA |
| | | | amidotransferase |
| | | | subunit B, |
| | | | putative |
| 47 | 0561 | 2.5 | hypothetical |
| | | | protein |
| 48 | 0408 | 2.9 | hypothetical |
| | | | protein |
| 49 | 0987 | 3.0 | hypothetical |
| | | | protein |
| 50 | 0397 | 3.1 | Alpha- |
| | | | aminoacylpeptide |
| | | | hydrolase, |
| | | | putative |
| 51 | 0144 | 3.7 | hypothetical |
| | | | protein |
| 52 | 0256 | 4.1 | hypothetical |
| | | | protein |
| 53 | 0575 | 5.2 | hypothetical |
| | | | protein, |
| | | | conserved |
| 54 | 0539 | 6.5 | hypothetical |
| | | | protein |
| 55 | 0438 | 4.3 | hypothetical |
| | | | protein, |
| | | | conserved |

### EXAMPLE 2

### Therileria parva Antigens for Expression in Viral Vectors

To demonstrate the efficacy of different experimental vaccines based upon a increase of antigen-specific cell mediated immunity and a protection against experimental challenge, t gene candidates selected on bioinformatics and CTL screening (methods described in Examples 14-16) and are listed in Table 2.

TABLE 2

| | | | | | Limits | |
|---|---|---|---|---|---|---|
| | | | | | of | BoLA |
| | | | | Signal | Signal | classI |
| Name | Origin | Identity | Size | Peptide | Peptide | haplotype |
| Tp1 | cDNA | unknown | 61 kDa | Yes | 1-19 | A18 |
| Tp2 | Genome + | unknown | 19 kDa | Yes | 1-23 | T2a |
| | CTL | | | | | |
| Tp3 | Genome + | unknown | 29 kDa | Yes | 1-16 | |
| | CTL | | | | | |
| Tp4 | cDNA | TCP-1 | 63 kDa | No | | A10 |
| | | (chaperone) | | | | |
| Tp5 | cDNA | EIF-1a | 18 kDa | No | | T5 |
| Tp6 | Genome + | Prohibitin | 31 kDa | Yes | 1-29 | |
| | CTL | | | | | |
| Tp7 | cDNA | Hsp90 | 84 kDa | No | | |
| | | (chaperone) | | | | |
| Tp8 | cDNA | Cysteine | 50 kDa | Yes | | A10 |
| | | protease | | (anchor) | | |

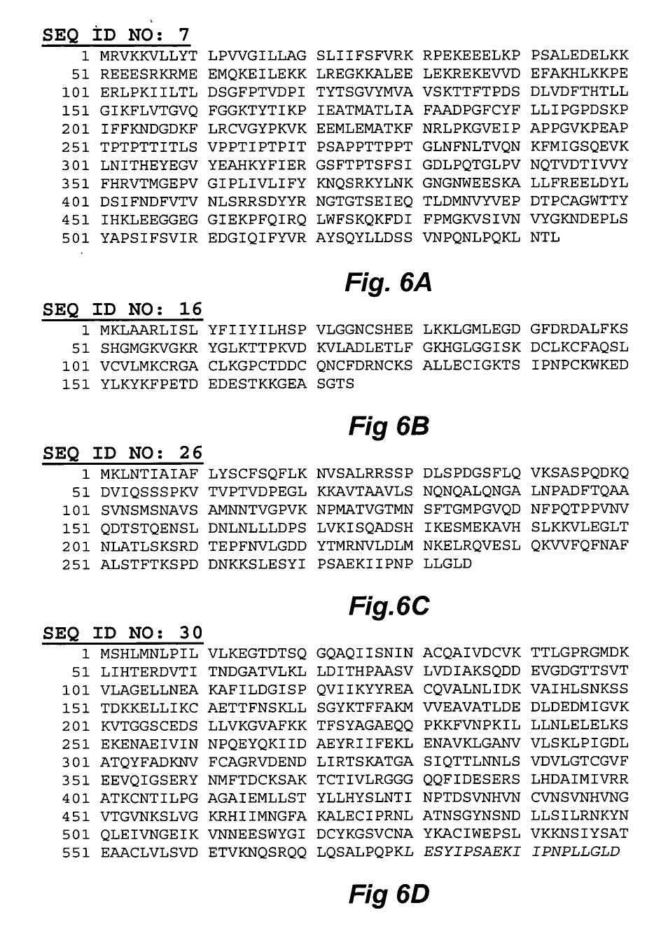

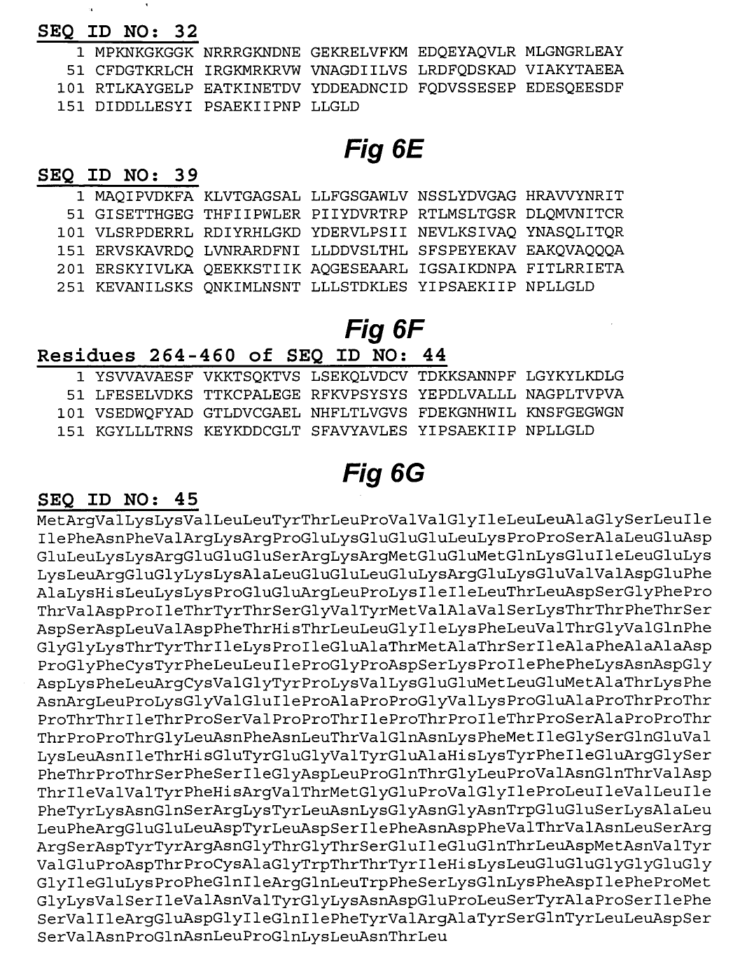

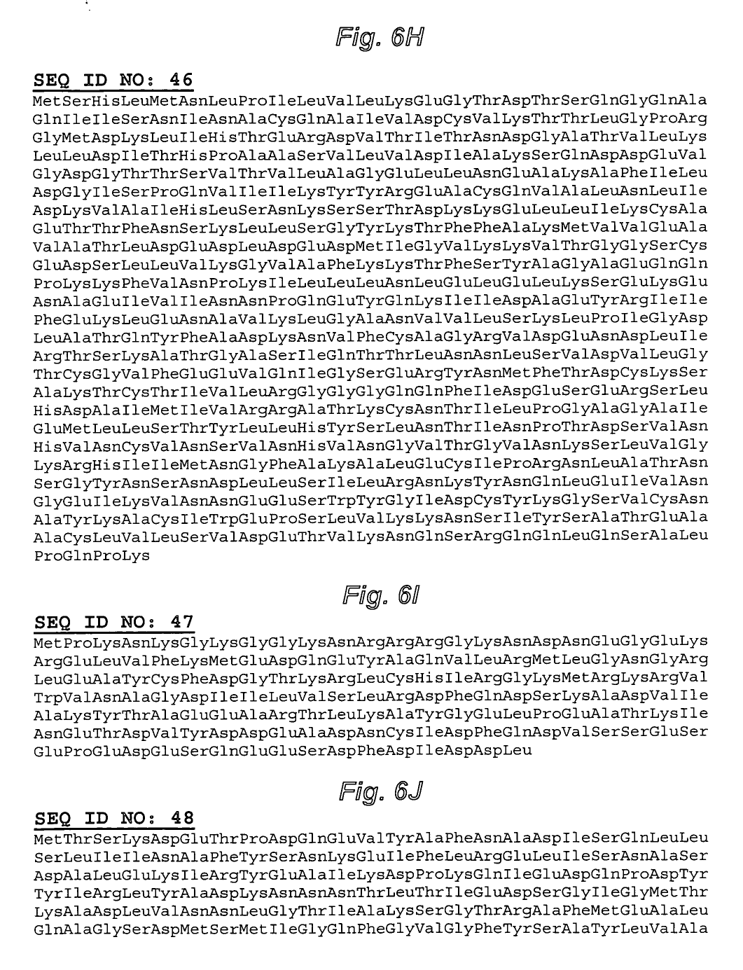

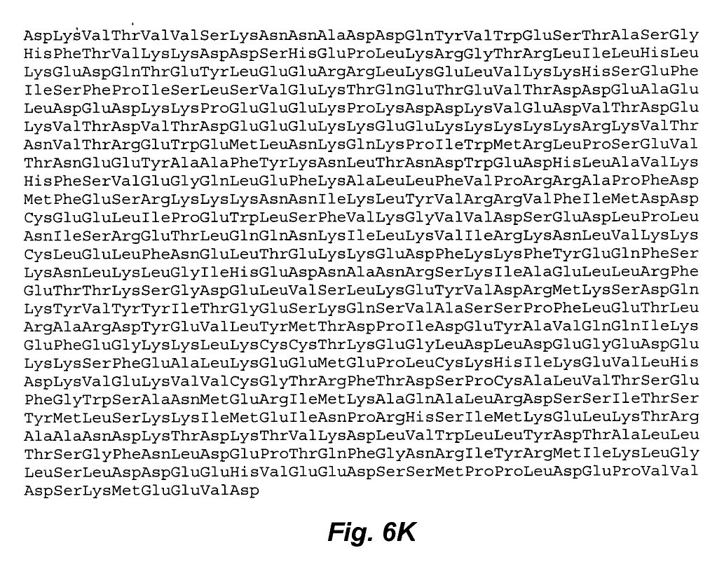

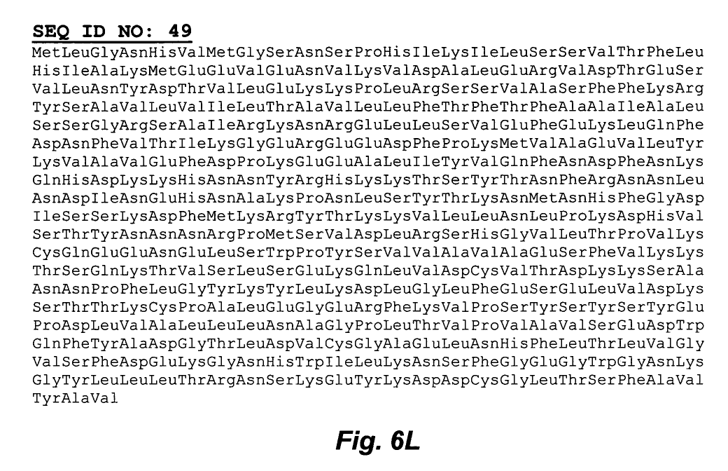

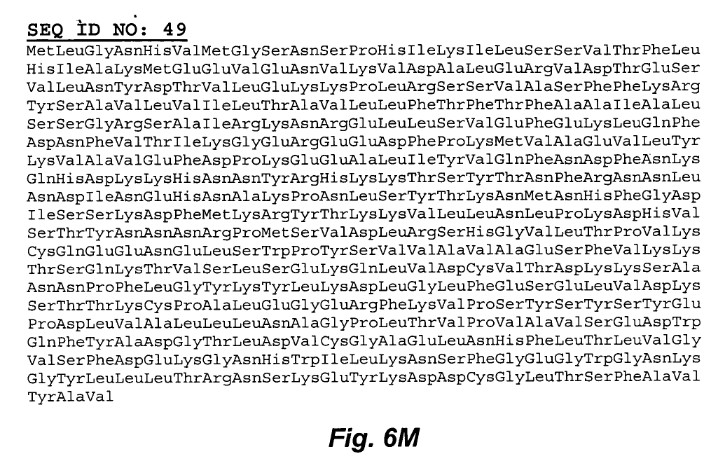

The amino acid sequence of Tp1 (SEQ ID NO 45) encoded by SEQ ID NO: 50 is shown in FIG. 6H, the amino acid sequence of Tp4 (SEQ ID NO 46) encoded by SEQ ID NO: 51 is shown in FIG. 6I, the amino acid sequence of Tp5 (SEQ ID NO 47) encoded by SEQ ID NO: 52 is shown in FIG. 6J, the amino acid sequence of Tp7 (SEQ ID NO 48) (GenBank Accession No. P24724) encoded by SEQ ID NO: 53 is shown in FIG. 6L and the amino acid sequence of Tp8 (SEQ ID NO: 49) encoded by SEQ ID NO: 54 is shown in FIG. 6M.

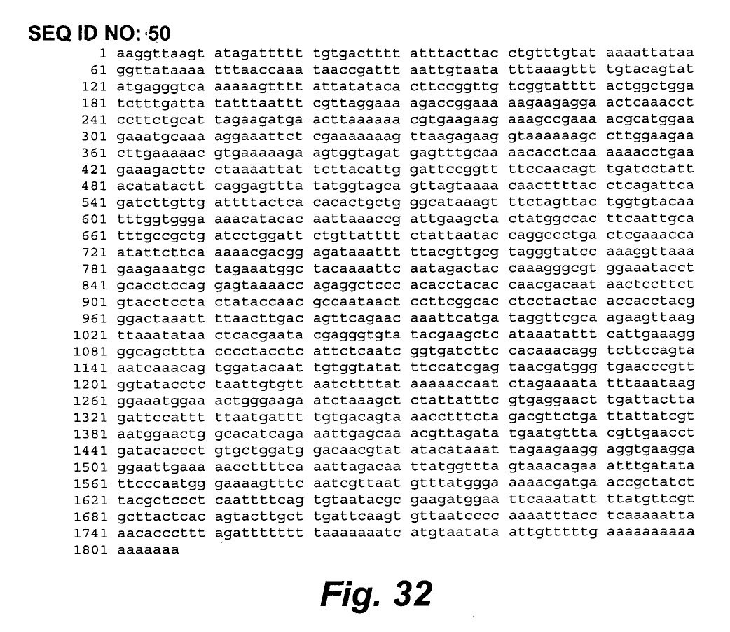

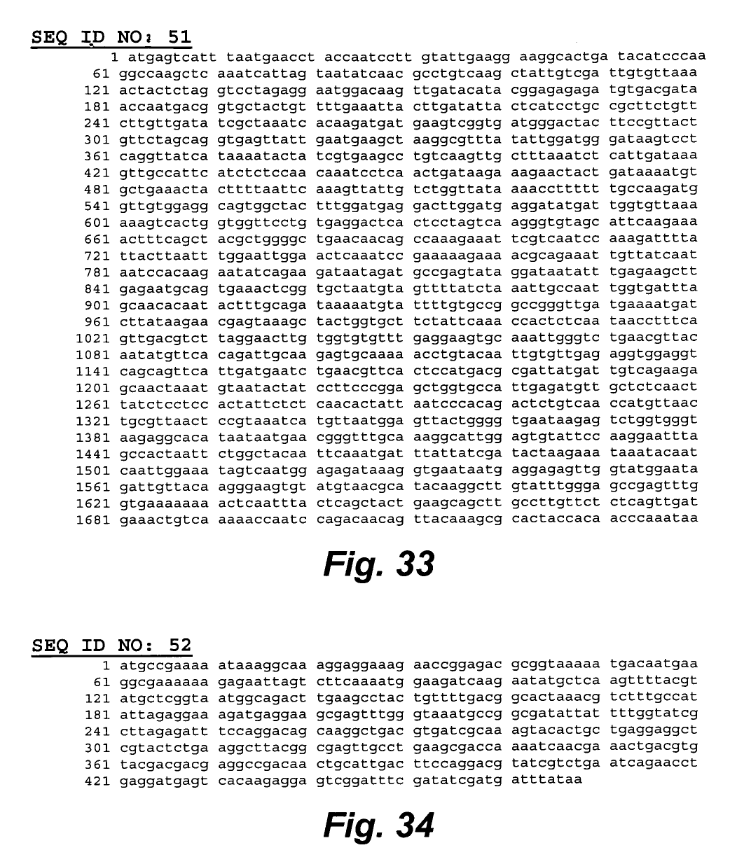

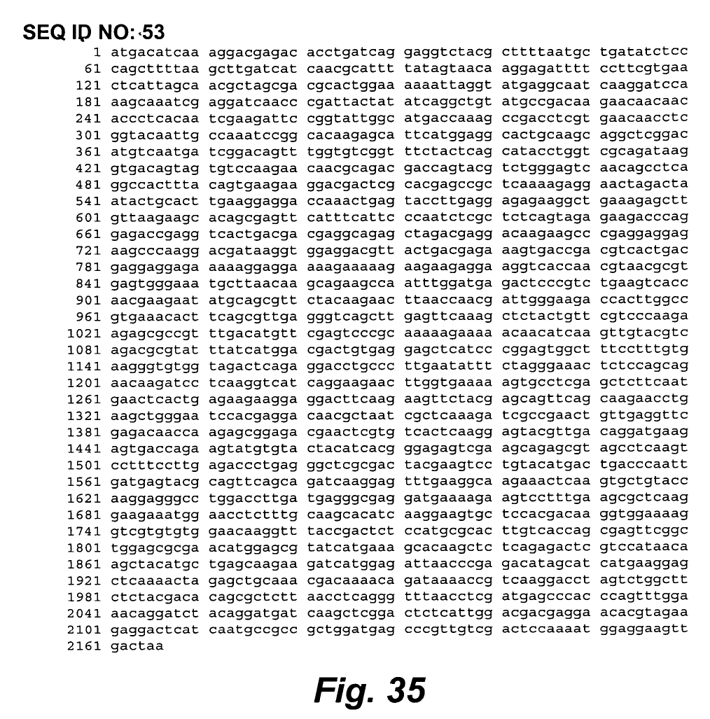

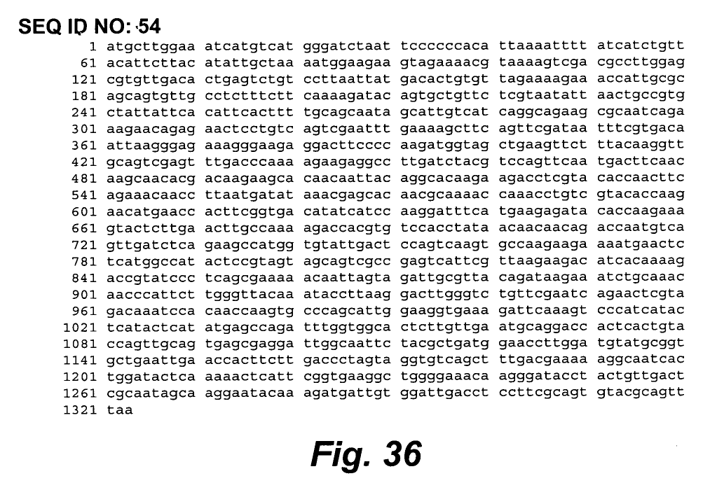

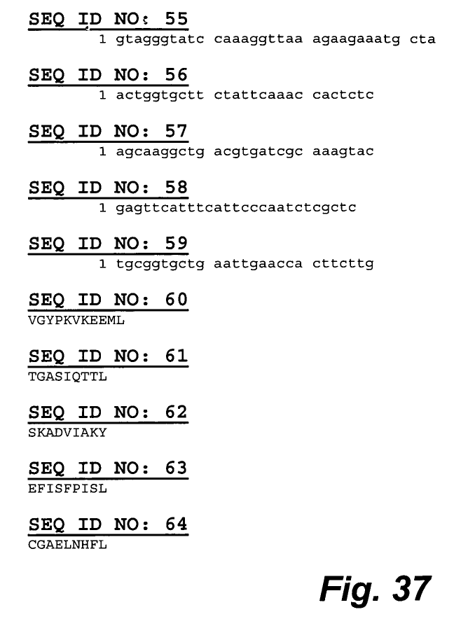

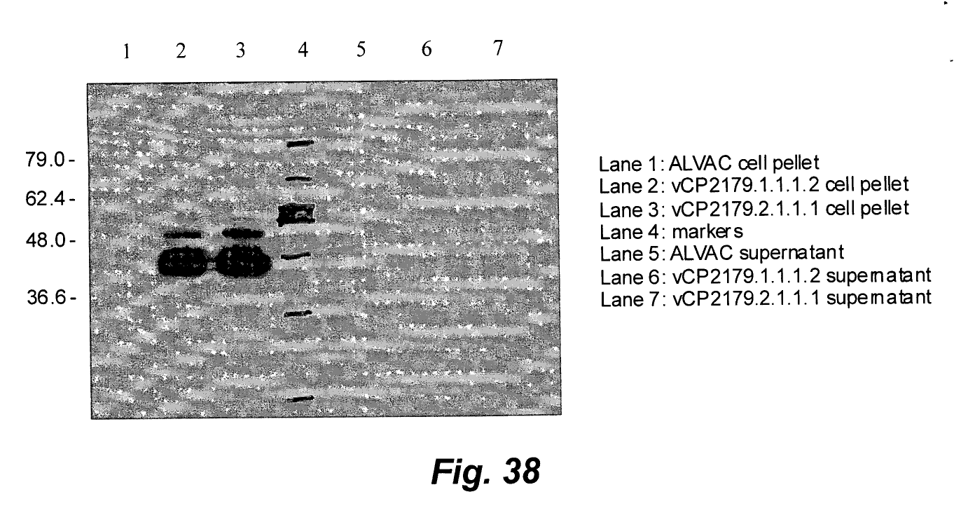

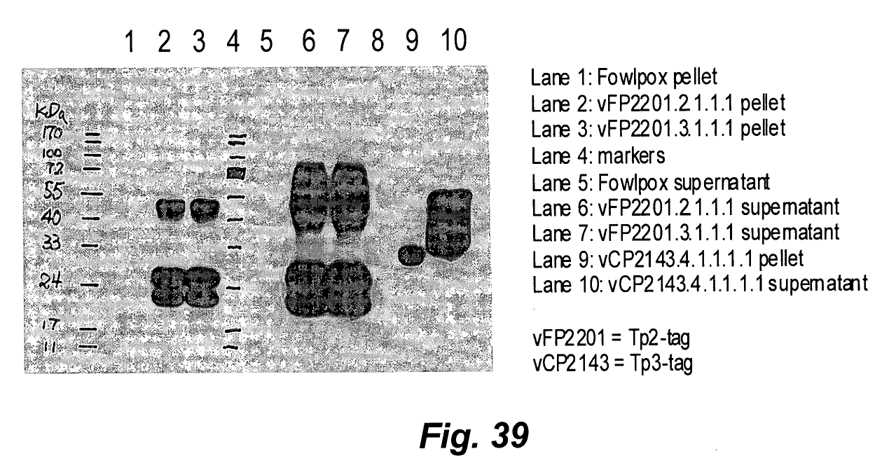

The nucleotide sequence SEQ ID NO: 50 (GenBank Accession No. CS008930) encoding the amino acid sequence ofTp1 (SEQ ID NO 45) is shown in FIG. 32, the nucleotide sequence SEQ ID NO: 51 (GenBank Accession No. CS008931) encoding the amino acid sequence of Tp4 (SEQ ID NO 46) is shown in FIG. 33, the nucleotide sequence SEQ ID NO: 52 (GenBank Accession No. CS008932) encoding the amino acid sequence of Tp5 (SEQ ID NO 47) is shown in FIG. 34, the nucleotide sequence SEQ ID NO: 53 (GenBank Accession No. CS008933) encoding the amino acid sequence of Tp7 (SEQ ID NO 48) is shown in FIG. 48 and the nucleotide sequence SEQ ID NO: 54 (GenBank Accession No. CS008934) encoding the amino acid sequence of Tp8 (SEQ ID NO: 49) is shown in FIG. 49.

### EXAMPLE 3

### Cloning of Targeted Genes

All the constructions are implemented using standard molecular biology methods (cloning, digestion by restriction enzymes, synthesis of a complementary single-strand DNA, polymerase chain reaction, elongation of an oligonucleotide by DNA polymerase, etc.) described, for example, by Sambrook J. et al. (Molecular Cloning: A Laboratory Manual, 2nd edition, Cold Spring Harbor Laboratory, Cold Spring Harbor. N.Y., 1989).

Open reading frames (ORFs) less than 3.1 kb were amplified by OneStep RT-PCR kit (QIAGEN Ltd., Crawley, UK) using RNA purified from T. parva (Muguga) schizont-infected lymphoblasts. PCR thermal cycles were (for genes up to 1 kb): 50° Celsius, 30 mins; 95° Celsius, 15 mins; 94° Celsius, 30 secs; 55° Celsius, 30 secs; 72° Celsius, 1 min.; 35 times from 94° Celcius cycle and finally 72° Celsius, 10 mins For genes between 1 and 2 kb conditions were: 50° Celsius, 30 mins; 95° Celsius, 15 mins; 94° Celsius, 1 minute; 55° Celsius, 1 minute; 72° Celsius, 1 minute; 35 times from 94° Celsius cycle and finally 72° Celsius, 10 mins. Genes between 2 and 3 kb: 45° Celsius 30 mins; 95° Celsius, 15 mins; 94° Celsius, 10 secs; 55° Celsius, 1 minute; 68° Celsius, 3 mins; 35 times from 94° Celsius cycle and finally 68° Celsius 10 mins. Amplified genes were purified from agarose gels by QlAquick Gel Extraction kit (QIAGEN) and cloned into the eukaryotic expression T-vector pTargeT (Promega, Madison, Wis., USA). Ligated samples were electroporated into host E. coli JM109, and colonies were screened by PCR using gene specific internal forward primers and vector specific reverse primer SEQ ID NO: 66 (5′-GAGCGGATAACATCACACAGG-3′). Plasmids from positive colonies were purified by QIAprep Spin Miniprep kit (QIAGEN) and sequenced by ABI PRISM 377 DNA sequencer (Applied Biosystems, Foster City, Calif., USA).

### EXAMPLE 4

### Construction of Recombinant ALVAC Constructs

The coding sequence of a mouse B-cell epitope of SV5 P/V protein, Pk-Tag, was PCR-amplified from pSC11-85A using XhoTag forward primer (SEQ ID NO: 67) 5′-CTCGAGTCCTACATACCATCTGCCGAAAAG-3′ and Tag reverse primer (SEQ ID NO: 68) 5′-TCAGTCTAGTCCTAGCAAAGGG-3′. The amplified fragment was cloned into pGEM-T Easy vector (Promega) to make pGEMTag. ORFs of T. parva polypeptides Tp1, Tp2, Tp4, Tp5 and Tp8 excluding the stop codons were amplified by OneStep RT-PCR (Qiagen) using Tp-gene specific primers and RNA extracted from purified schizonts. Amplified fragments were purified by QIAquick PCR Purification Kit (Qiagen), treated with T4 DNA polymerase and digested with Xho I. PCR fragments were ligated with pGEMTag, which was digested with Spe I and treated with Klenow enzyme, then digested with Xho I to form pGEMTp1Tag, pGEMTp2Tag, pGEMTp4Tag, pGEMTp5Tag, and pGEMTp8Tag. These plasmids were used as templates to amplify Tp-Tag fusion genes by PCR using Tp gene-specific forward primers and Tag reverse primer. PCR fragments were purified from agarose gels by QIAquick Gel Extraction Kit (Qiagen), treated with T4 DNA polymerase and cloned into pSG2 DNA vaccine plasmids (McConkey et al., (2003) Nat. Med. 9: 729) to form pSG2Tp1Tag, pSG2Tp2Tag, pSG2Tp4Tag, pSG2Tp5Tag, pSG2Tp8Tag. All plasmids were sequenced and the expression of Tp antigens were confirmed by immunostaining of transfected COS-7 cells (ATCC CRL-1651) with anti-V5-TAG mouse monoclonal antibody MCA1360P (Serotec, Oxford, UK).

Recombinant Canarypox Virus (vCP) shuttle vectors were prepared by cloningTp1Tag, Tp2Tag, Tp4Tag, Tp5Tag and Tp8Tag PCR fragments into a pC5H6p donor plasmid to form pC5H6p-Tp1Tag, pC5H6p-Tp2Tag, pC5H6p-Tp4Tag, pC5H6p-Tp5Tag, pC5H6p-Tp8Tag. Primary chicken embryo fibroblasts (CEF) were infected with wild-type canarypox virus (ALVAC strain) and transfected with pC5H6p-TpTag plasmids. Positive plaques were selected on the basis of hybridization with an HRP-labeled probe specific to the Tp sequence. These plaques underwent 2-4 successive selection/purification cycles until a pure population was isolated. A representative plaque corresponding to in vitro recombination between the donor plasmid and the genome of the ALVAC canarypox virus was then amplified and the recombinant virus stock obtained was designated vCPTp1Tag, vCPTp2Tag, vCPTp4Tag, vCPTp5Tag, and vCPTp8Tag, respectively.

Expression of Tp antigens was confirmed by immunostaining of viral plaques with anti-V5-TAG mouse monoclonal antibody. Virus stocks were prepared by infecting CEFs and titrated by plaque assay on CEF cells and stored at −80° Celsius. All vaccine constructs were screened for the existence of mycoplasma using a Mycoplasma Detection Kit Version 2.0 (ATCC) and bovine viral diarrhea virus (BVDV) by RT-PCR. Endotoxin concentrations in plasmid DNA preparations were measured using the QCL-1000 kit (BioWhittaker, Inc., Walkersville, Md., USA). Plasmid DNA was considered endotoxin-free if endotoxin was less than 100 EU/mg DNA.

A representative plaque corresponding to in vivo recombination between the donor plasmid and the genome of the ALVAC canarypox virus was then amplified to provide a recombinant virus stock. Specific cloning protocols and the resulting ALVAC clones are described in Examples 5-11 below.

Expression vector constructs using ALVAC as the vector are presented in Table 3 below.

TABLE 3

| Construct | |
|---|---|
| Vector-insert | Designation |
| ALVAC-Tp1 | vCP2112 |
| ALVAC-Tp1-tag | vCP2163 |
| ALVAC-Tp2 | vCP2110 |
| ALVAC-Tp3-tag | vCP2043 |
| ALVAC-Tp4-tag | vCP2164 |
| ALVAC-Tp5-tag | vCP2138 |
| ALVAC-Tp6-tag | vCP2137 |
| ALVAC-Tp8-tag | vCP2179 |

### EXAMPLE 5

### Construction of ALVAC Tp1, vCP2112

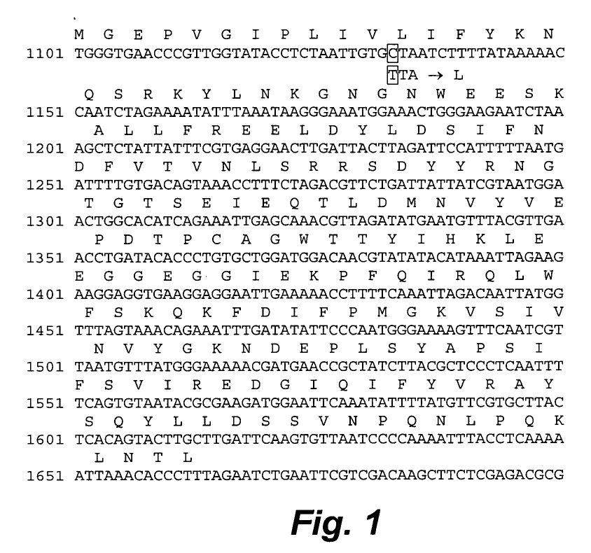

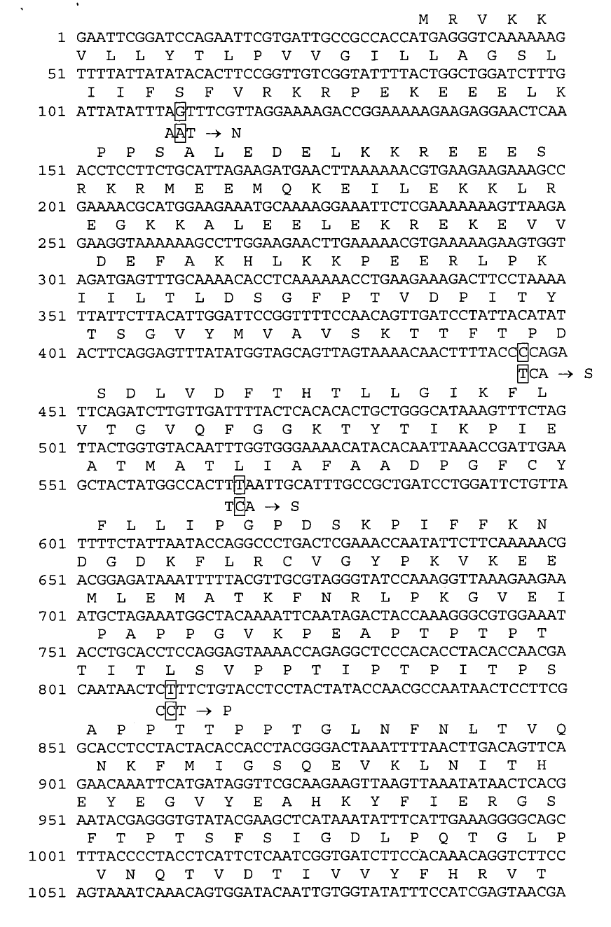

Plasmid construction: Plasmid pTargetTp1 contained theTp1 gene nucleotide sequence (SEQ ID NO: 1) from Theileria parva strain Muguga. There were five differences between the sequence as provided by The Institute for Genomic Research (TIGR) (identified by BLAST search) and the sequence of the p Target Tp1 template (SEQ ID NO: 1) as shown in FIG. 1. Four of these differences result in changed amino acids and one difference is silent.

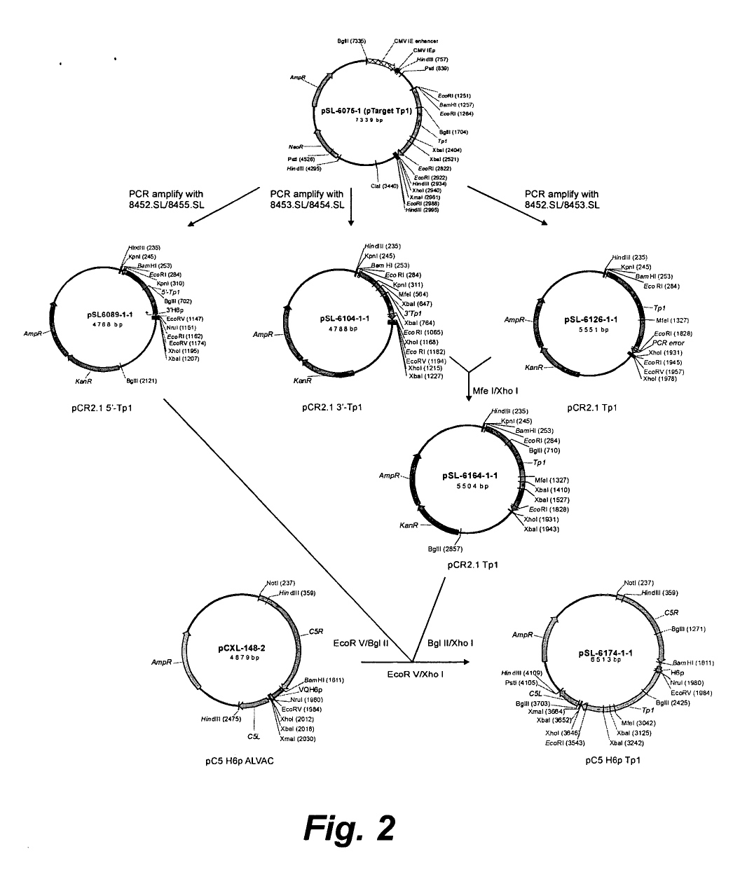

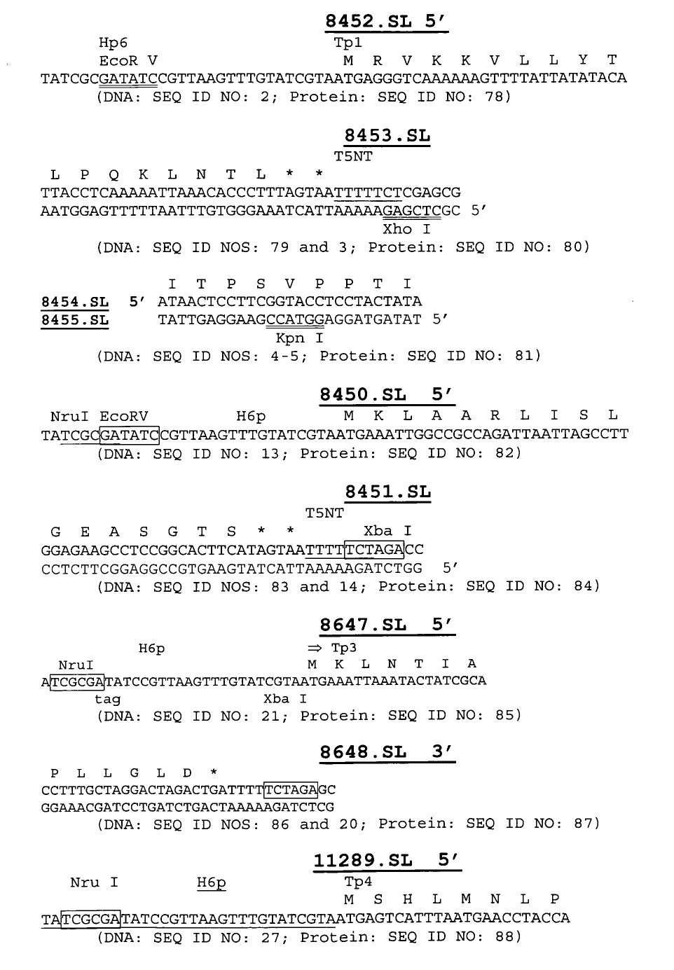

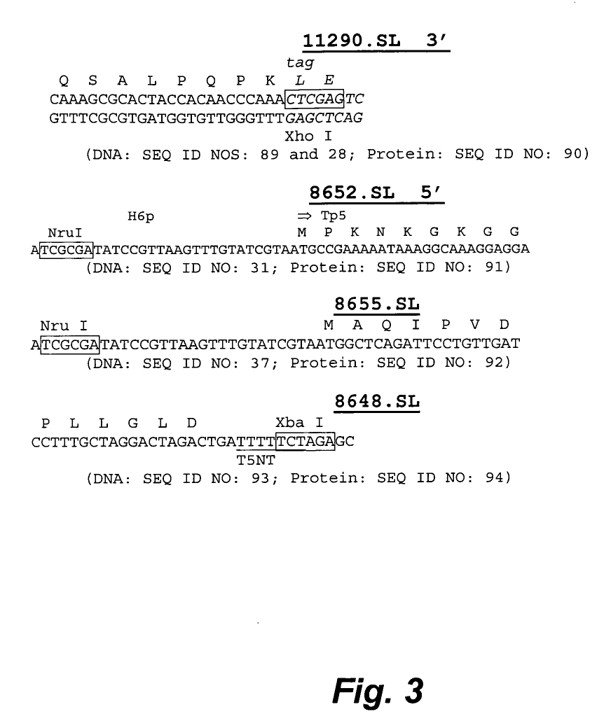

Plasmid p Target Tp1 was used to generate a C5 donor plasmid pC5 H6pTp1 (pSL-6174-1-1) containing the Theileria parva Tp1 gene. In pSL-6174-1-1, the plasmid backbone was pCXL-148-2 (pC5 ALVAC H6p) with theTp1 coding sequence SEQ ID NO: 1 between the EcoR V and Xho I sites, the vaccinia virus H6 promoter and the ampR gene. The plasmid construction is schematically illustrated in FIG. 2. PCR amplification of the 5′-Tp1 gene used pTargetTp1 plasmid as the template and primers 8452.SL (SEQ ID NO: 2) and 8455.SL (SEQ ID NO: 5) illustrated in FIG. 3. Primer 8452.SL (SEQ ID NO: 2) included the 3′-end of the H6 promoter, and primer 8455.SL (SEQ ID NO: 5) included a 3′-Kpn I site for later cloning. The resultant 0.8 kb PCR fragment was cloned into vector pCR2.1 to generate pSL-6089-1-1 (see FIG. 2), which had the correct sequence of the H6p promoterTp1, but with the desired Kpn I site not in the desired location.

PCR amplification of 3′-Tp1 used pTargetTp1 as template and primers 8453.SL (SEQ ID NO: 3) and 8454.SL (SEQ ID NO: 4) illustrated FIG. 3. Primer 8454.SL (SEQ ID NO: 4) introduced a 5′-KpnI site for later cloning and primer 8453.SL (SEQ ID NO: 3) introduced a stop signal, T5NT transcription terminator, and an Xho I site for cloning. The approximately 0.8 kb fragment was then cloned into pCR2.1, generating pSL-6104-1-1 (FIG. 2). The fragments from pSL-6089-1-1 and pSL-6104-1-1, however, could not be joined due to the misplaced Kpn I site in pSL-6089-1-1.

PCR amplification ofTp1 used pTargetTp1 as template and primers 8452.SL (SEQ ID NO: 2) and 8453.SL (SEQ ID NO: 3). The resultant approximately 1.6 kb fragment was cloned into pCR2.1, generating pSL-6126-1-1, which did not contain the promoter sequence and had a PCR error at the 3′-end.

The PCR-based error in pSL-6126-1-1 was corrected by replacing the 0.6 kb Mfe I/xho I fragment with the corresponding fragment from pSL-6104-1-1, resulting in pSL-6164-1-1. This plasmid contained the correctTp1 sequence, but had no H6 promoter.

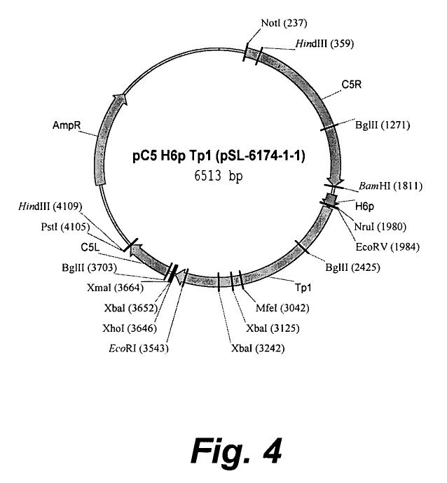

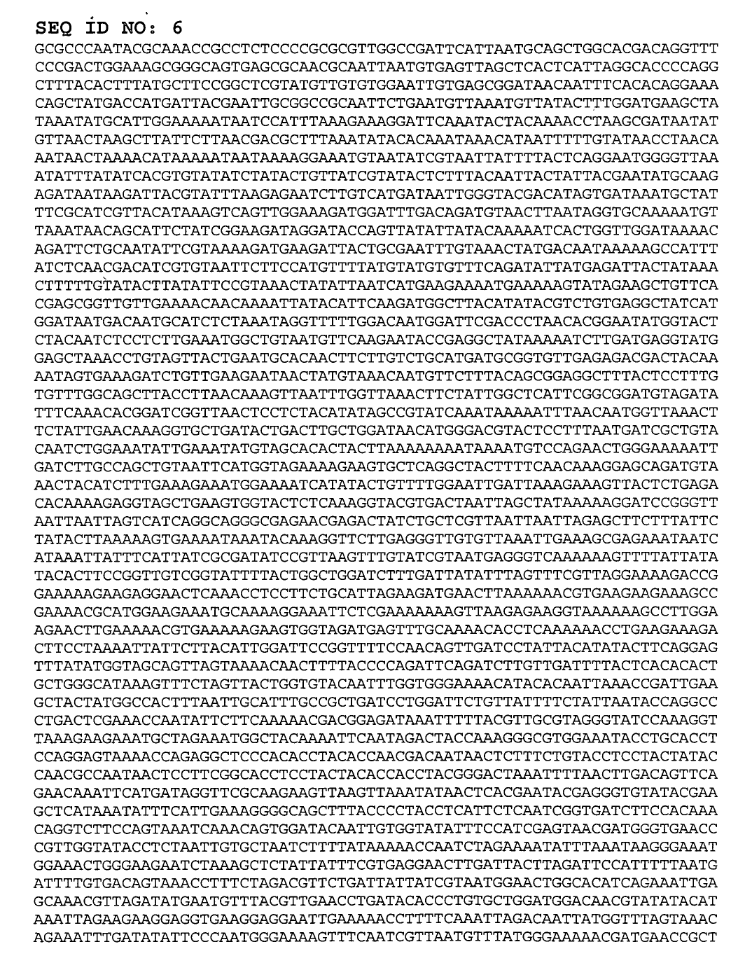

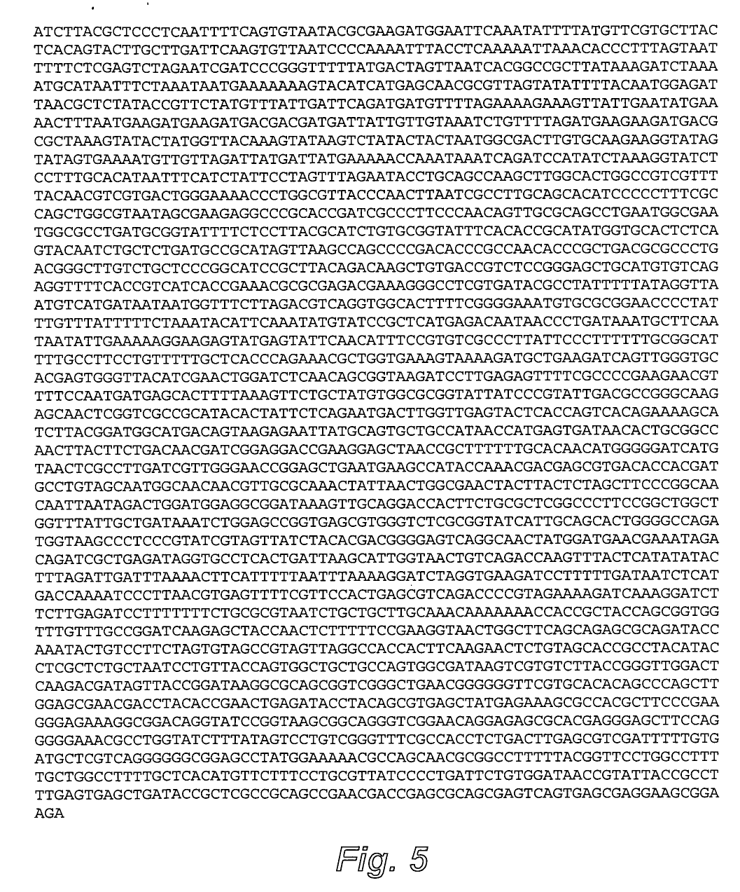

pCXL-148-2 is a C5 donor plasmid containing the H6 promoter. The 0.4 kb EcoR V-Bgl II fragment from pSL-6089-1-1 and the 1.2 kb Bgl II/Xho I fragment from pSL-6164-1-1 were ligated with the 4.9 kb EcoR V/Xho I vector fragment from pCXL-148-2 to generate pSL-6174-1-1 as shown in FIG. 4. The positions of the various regions of the 6.513 kb pSL-6174-1-1 sequence (SEQ ID NO: 6, as shown in FIG. 5) are: C5R: nucleotides 236-1809; H6p: nucleotides 1881-2004; Tp1: nucleotides 2005-3633; C5L: nucleotides 3696-4105; and ampR: 4712-5569. The predicted amino acid sequence of the product ofTp1 (SEQ ID NO: 7) is shown in FIG. 6A.

Preparation of a Tp1-specific probe: Primers for amplifying a Tp1-specific probe were: 8456.SL: 5′-ACTCACACACTGCTGGGCATAAAGTT-3′ (SEQ ID NO: 8); 8459.SL: 5′-GAACGTCTAGAAAGGTTTACTGTCAC-3′ (SEQ ID NO: 9) and primers for PCR amplification of the vCP2112 C5 arms plus insert were: 7931.DC: 5′-GAATCTGTTAGTTAGTTACTTGGAT-3′ (SEQ ID NO: 10) and 7932.DC 5′-TGATTATAGCTATTATCACAGACTC-3′ (SEQ ID NO: 11).

Cells for in vivo recombination: Primary chicken embryo fibroblast cells (CEFs) were grown in 10% fetal bovine serum (JRH: γ-irradiated cat # 12107 Lot# ON0469, 1L0232), DMEM (BRL/Gibco Cat #11960) supplemented with 4 mM glutamine (BRL/Gibco Cat #25030-081), 1 mM sodium pyruvate (BRL/Gibco Cat #11360-070) and 1×antibiotics/antimycotics (P/S/A/A, BRL/Gibco Cat #15240-062).

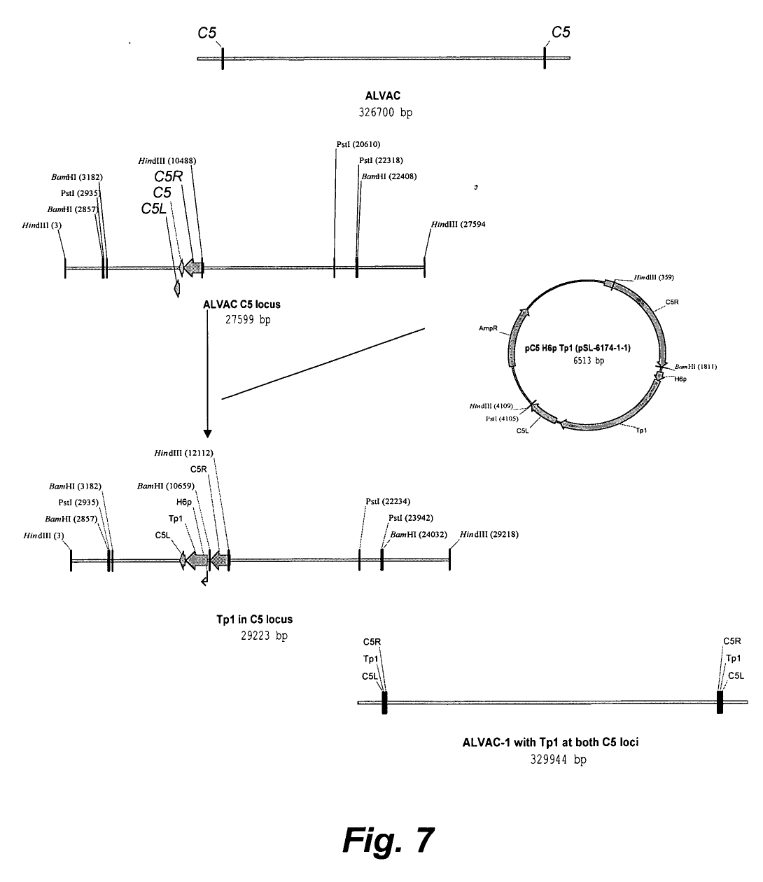

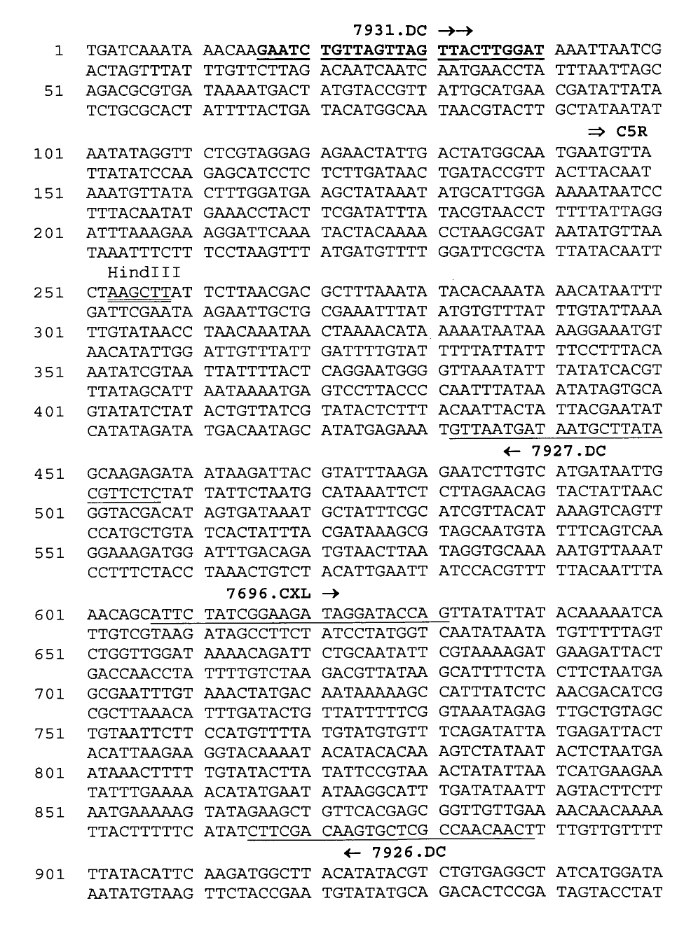

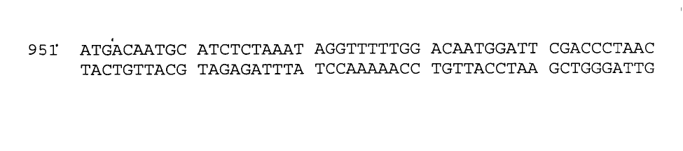

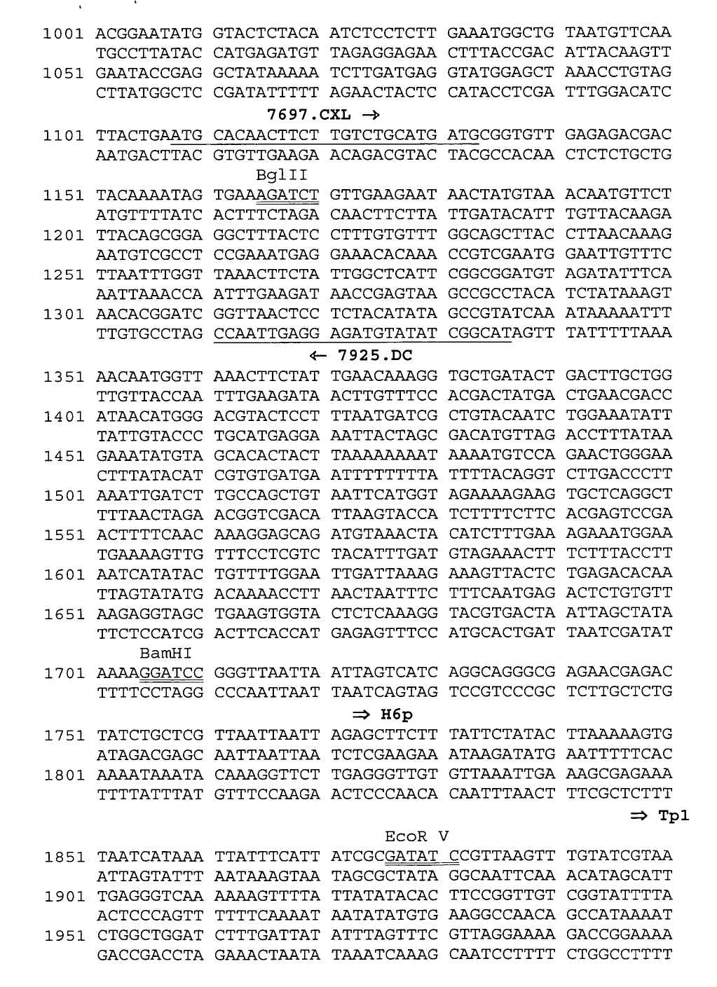

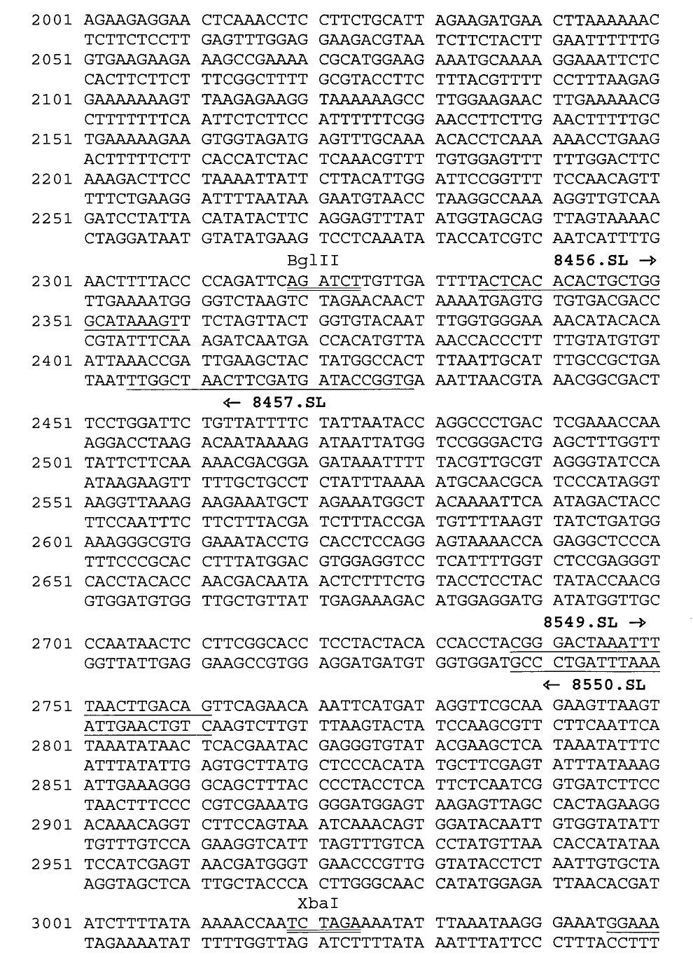

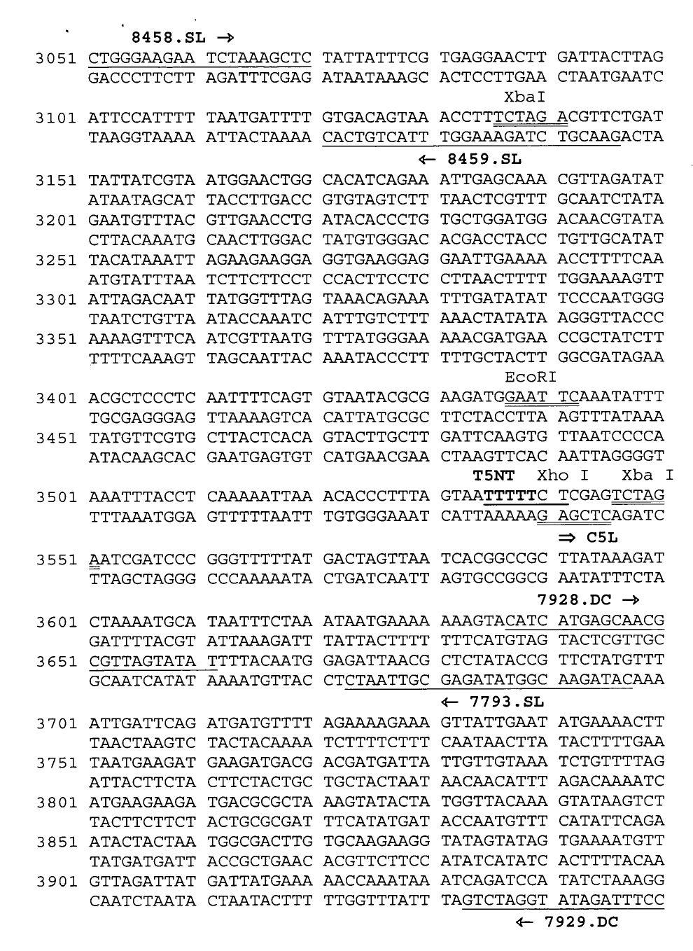

Recombinant generation: In vivo recombination, shown schematically in FIG. 7, was performed by transfecting primary CEF cells with Not I-linearized donor plasmid pSL-6174-1-1 (15 μg) using Fugene reagent (Roche). The transfected cells were subsequently infected with ALVAC as the rescue virus at a MOI of 10 (stock virus of 1.2×10¹⁰ pfu/ml). After 24 hrs, the transfected/infected cells were harvested, sonicated and used for recombinant virus screening. Recombinant plaques were screened by plaque lift hybridization using the Tp1-specific probe, prepared as described above, and labeled with horseradish peroxidase according to the manufacturer's protocol (Amersham Cat# RPN3001). After four sequential rounds of plaque purification, the recombinants designated as vCP2112.7.2.1.3 and vCP2112.8.1.1.1 respectively were isolated and confirmed by hybridization as having theTp1 insert and negative for the C5 ORF. Single plaques of both isolateswere selected from the fourth round of plaque purification, and expanded to obtain P1 (T-25 flask), P2 (T75 flask) and P3 (roller bottle) stocks.

The infected cell culture fluids from the roller bottles were harvested and concentrated to produce virus stocks (2.2 ml of vCP2112.7.2.1.3 at 9.8×10⁹ pfu/ml and 2.2 ml of vCP2112.8.1.1.1 at 1.1×10¹⁰ pfu/ml).

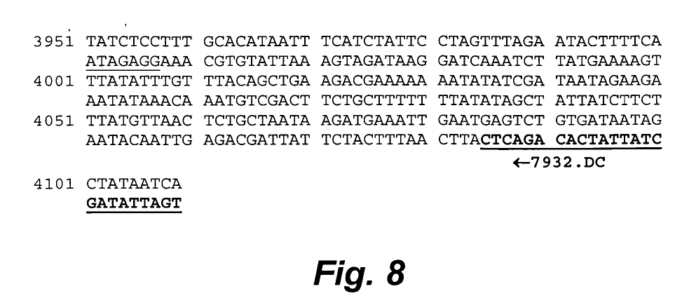

Sequence analysis: Analysis of viral stock genomic DNA was performed by PCR amplification and sequence analysis of the flanking arms of the C5 locus and the Tp1 insert. Primers 7931.DC 5′-GAATCTGTTAGTTAGTTACTTGGAT-3′ (SEQ ID NO: 10) and 7932.DC 5′-TGATTATAGCTATTATCACAGACTC-3′ (SEQ ID NO: 11) located beyond the arms of the C5 locus in the donor plasmid were used to amplify the entire C5R-Tp1 insert-C5L fragment. The nucleotide sequence SEQ ID NO: 12, and the sequence of the complementary strand thereof, of the recombinant ALVAC-Tp1 construct and the various domains therein is illustrated in FIG. 8.

### EXAMPLE 6

### Generation of ALVAC Recombinant Tp2, vCP2110

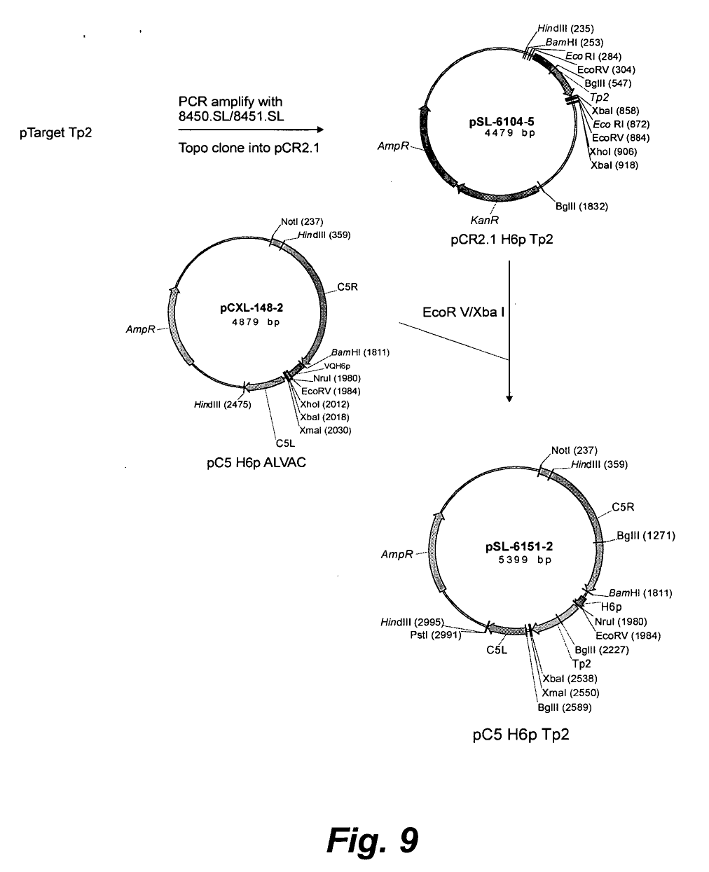

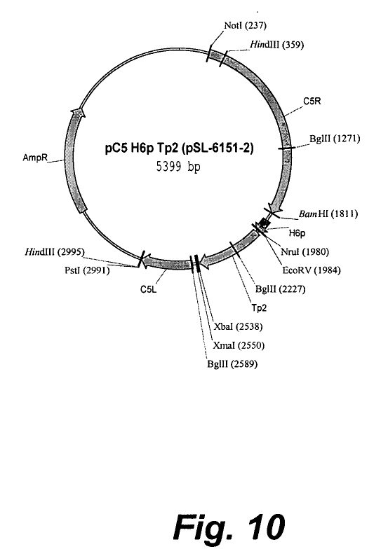

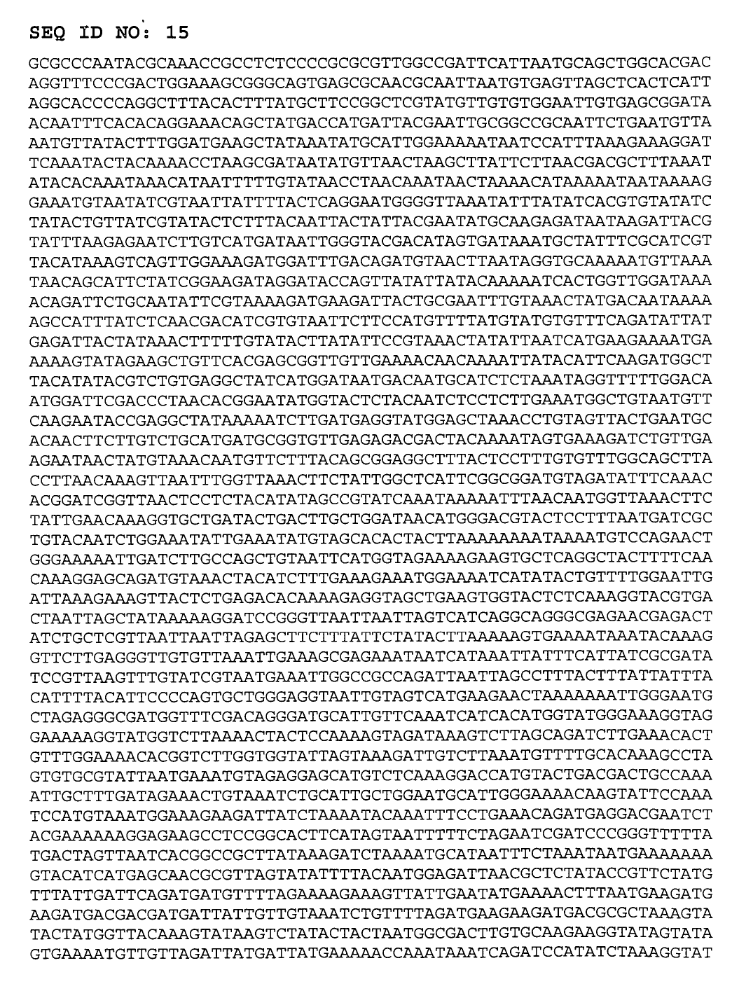

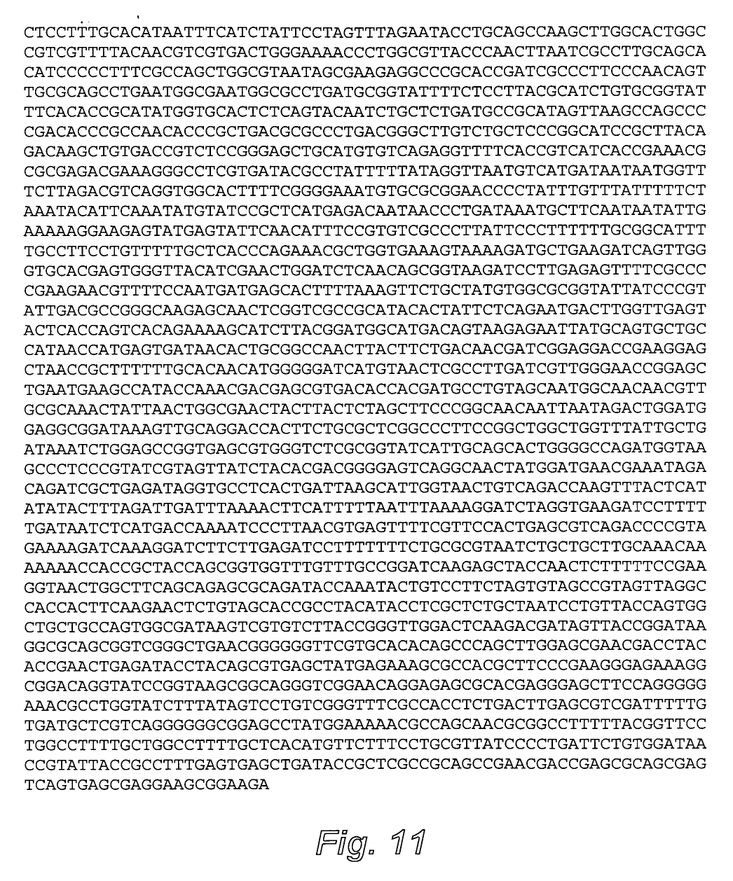

Plasmid construction: The Tp2 gene was PCR amplified using pTarget Tp2 as the template and primers 8450.SL (SEQ ID NO: 13) and 8451.SL (SEQ ID NO: 14), shown in FIG. 3, which introduced the 3′-end of the H6 promoter and the T5NT pox virus transcription termination signals respectively. The amplified fragment was cloned, as shown schematically in FIG. 9, into vector pCR2.1 and clone pSL-6104-5 was confirmed by sequence analysis. Plasmid pCXL-148-2 was digested with EcoR V and Xba I and ligated with the 0.6 kb EcoR V/Xba I fragment from pSL-6104-5. Clone pSL-6151-2, the map of which is shown in FIG. 10, was confirmed by sequence analysis of the C5 arms, H6 promoter and Tp2 insert. Within the nucleotide sequence of plasmid pSL-6151-2 (SEQ ID NO: 15 shown in FIG. 11), the nucleotide positions of identifiable regions are: C5R, 236-1809; C5L, 2582-2991; H6p, 1881-2004; Tp2, 2005-2526. The predicted amino acid sequence (SEQ ID NO: 16) of the Tp2 gene is shown in FIG. 6B.

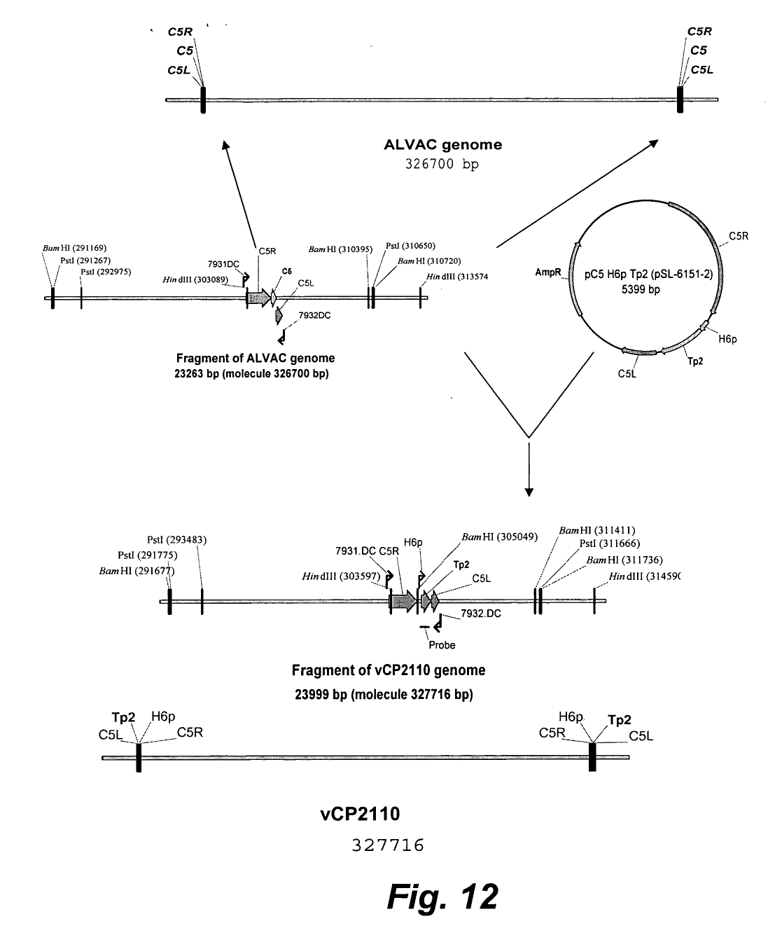

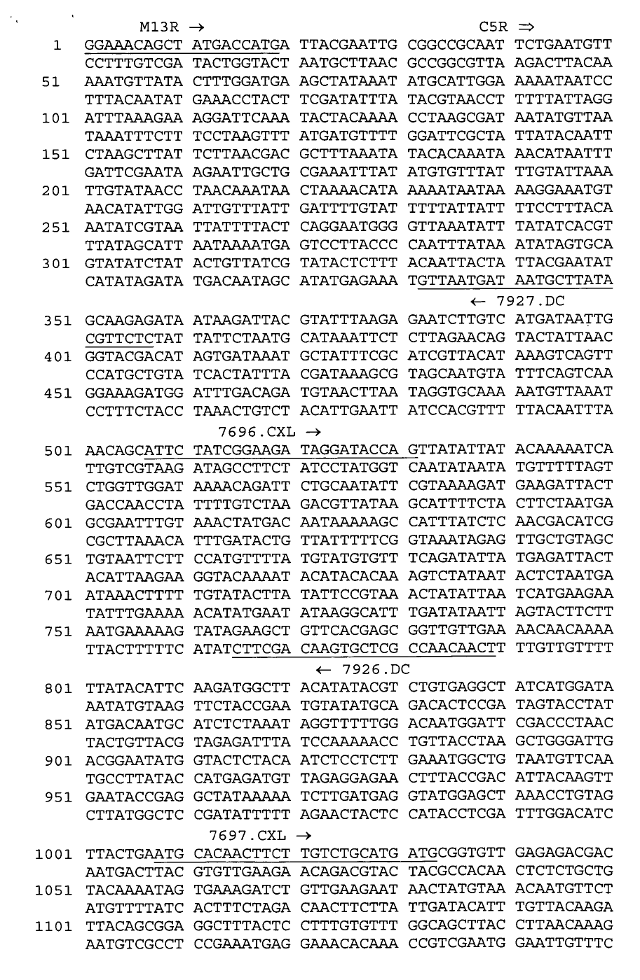

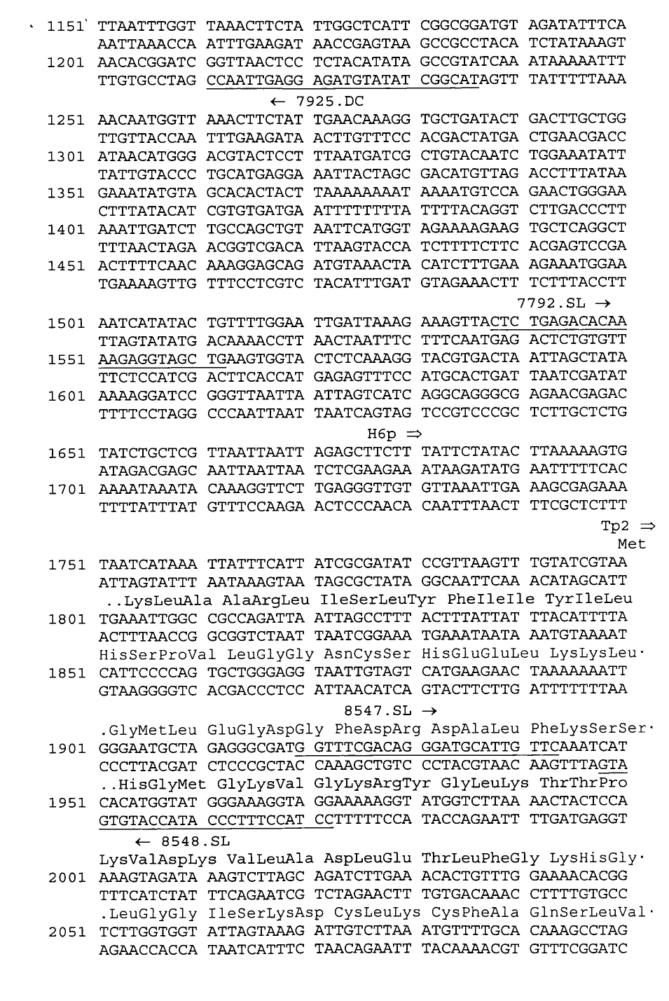

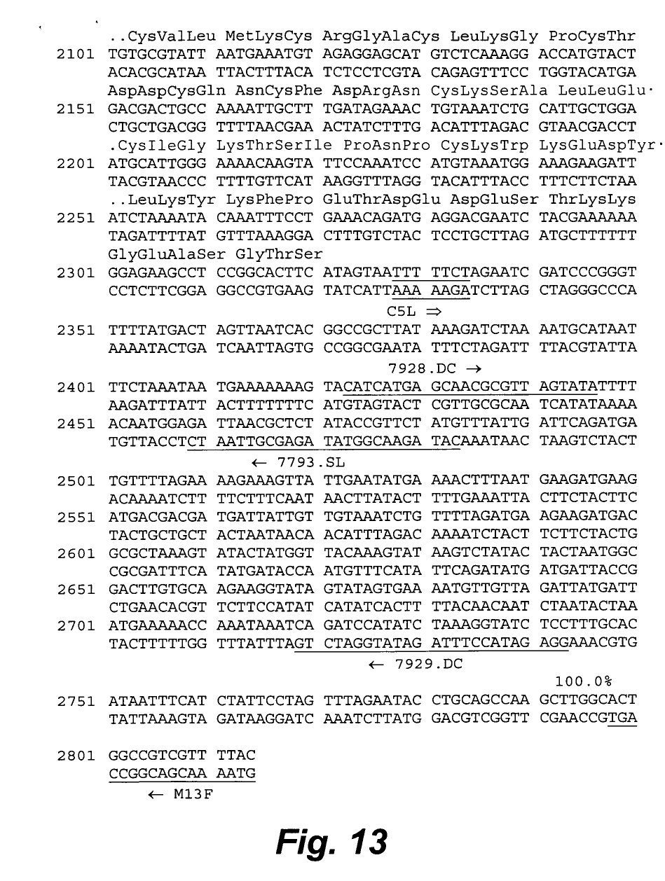

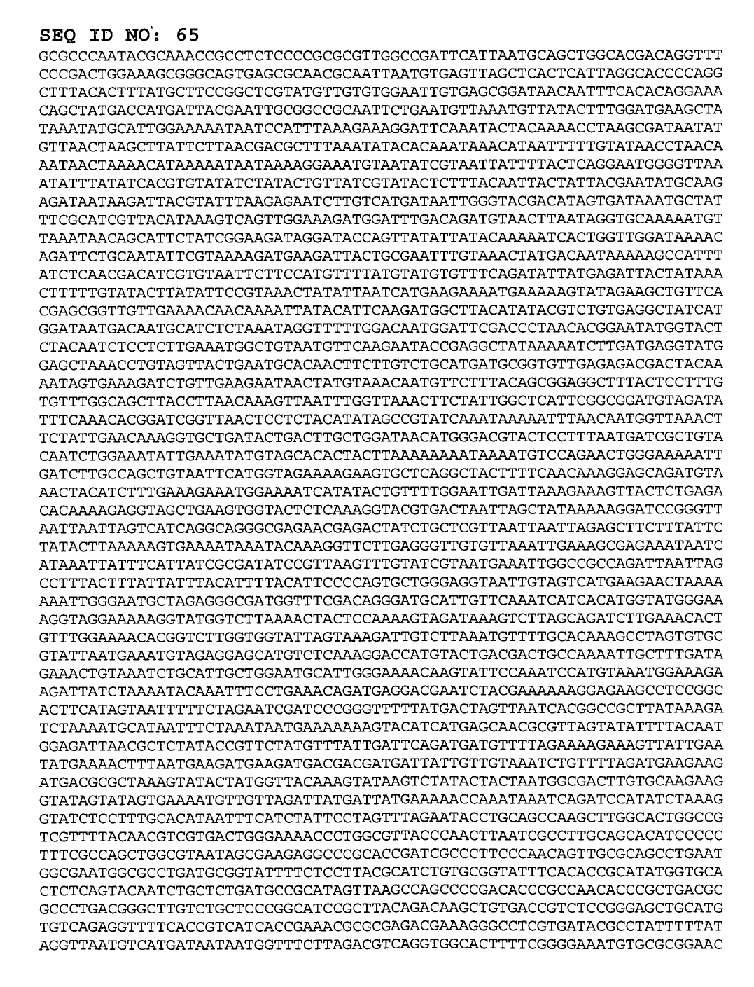

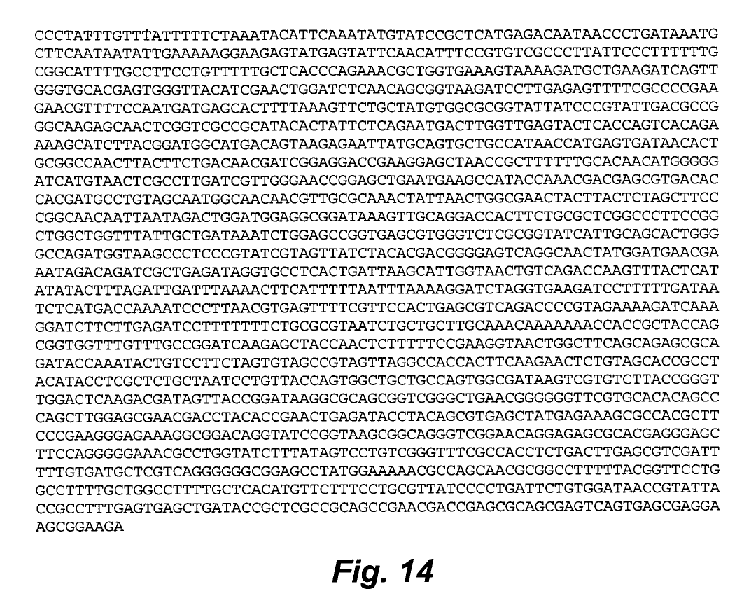

Recombinant generation: In vivo recombination was performed as described in Example 1 above and as shown schematically in FIG. 12. After 4 sequential rounds of plaque purification, recombinants designated vCP2110.1.1.1.1 and vCP2110.12.2.1.2 were isolated. Single plaques from the 4th round of plaque purification were expanded to obtain P1 (60 mm), P2 (T75 flask) and P3 (roller bottle) stocks to amplify vCP2110. Recombinants were re-confirmed at the P2 level by hybridization as positive for the insert and negative for the C5 ORF. The infected cell culture fluid from the roller bottles was harvested and concentrated to produce virus stock (2.25 ml of vCP2110.1.1.1.1 at 1.5×10¹⁰ pfu/ml and 2.25 ml of vCP2110.12.2.1.2 at 1.7×10¹⁰ pfu/ml).

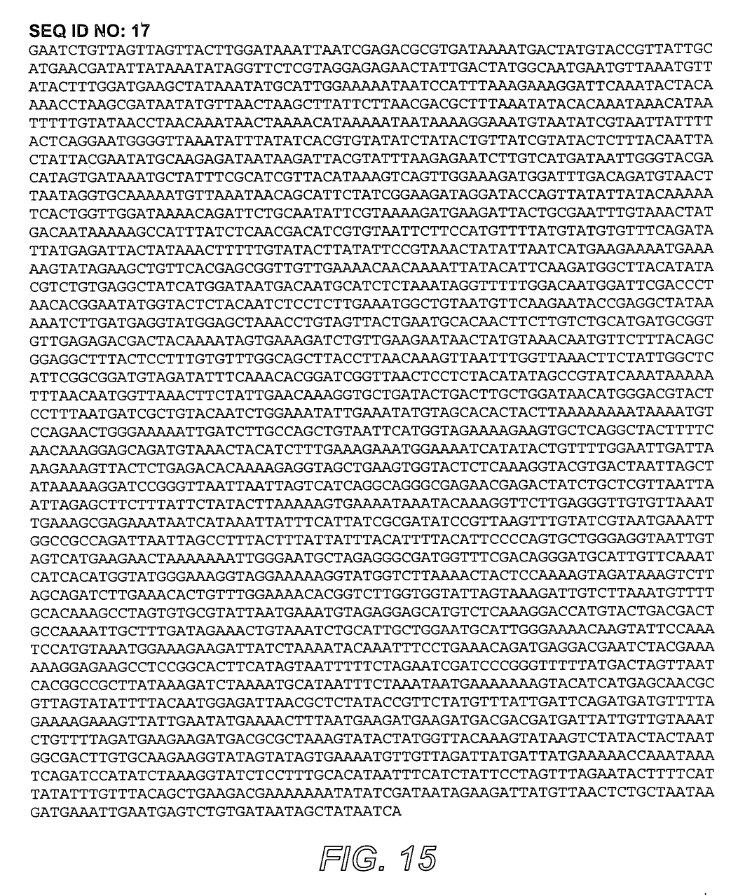

Analysis of the vCP2110 genomic DNA was performed by PCR amplification and sequence analysis of the flanking arms of the C5 locus and the Tp2 insert. Primers 7931.DC 5′-GAATCTGTTAGTTAGTTACTTGGAT-3′ (SEQ ID NO: 10) and 7932.DC 5′-TGATTATAGCTATTATCACAGACTC-3′ (SEQ ID NO: 11) were used to amplify the entire C5R-H6p Tp2-C5L fragment. The results showed that the sequences of the H6p Tp2 insert and the C5 left and right arms around the H6p Tp2 insert (SEQ ID NO: 17 as shown in FIG. 15) in the ALVAC vector were as expected, with the nucleotide positions therein as follows: Tp2, 1883-2404; Probe, 1853-2302; C5R, 126-1661; C5L, 2459-2863; H6 promoter, 1759-1882.

Primers for amplifying the Tp2-specific probe were 8565.DT: 5′ ACTTGTTTTCCCAATGCATTCCAG 3′ (SEQ ID NO: 18) and 8450.SL (SEQ ID NO: 13).

### EXAMPLE 7

### Construction of ALVAC Tp3, vCP2143

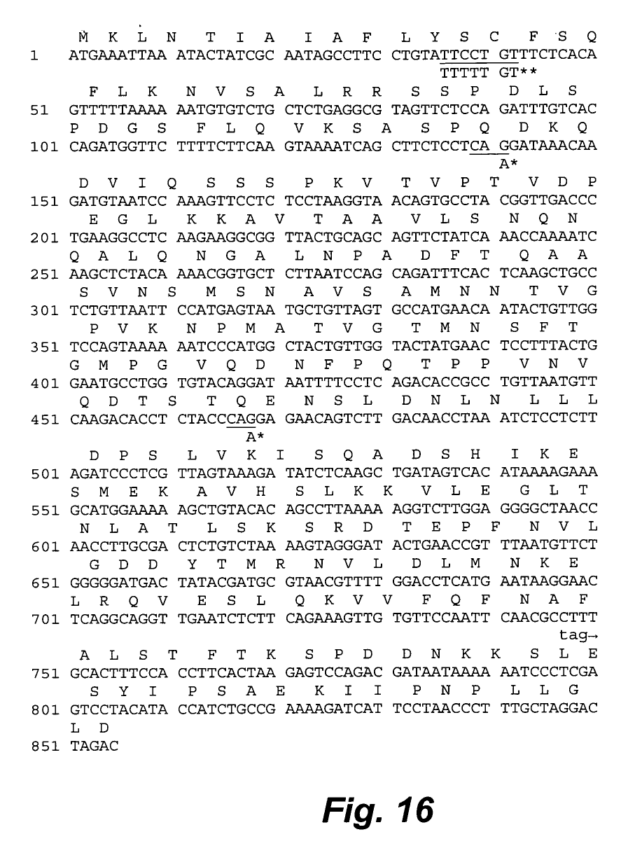

Plasmid construction: The insert of plasmid pSG Tp3-tag#5 encodes the T. parva gene Tp3 and a marker tag from the circumsporozoite protein of Plasmodium berghei and has the nucleotide sequence SEQ ID NO: 19 as shown in FIG. 16 was used. Within theTp1 coding sequence, one internal T5NT at the 5′ end of gene was removed by site directed mutagenesis (TTTTTGT→TTCCTGT). Two silent mutations relative to the TIGR sequence were found. This target plasmid was used to generate a C5 donor plasmid pC5 H6pTp1 (pSL-6174-1-1) containing the Theileria parva Tp3 gene. In pSL-6174-1-1, the plasmid backbone was pCXL-148-2 (pC5 ALVAC H6p) having the insertion of Tp3 between the EcoR V and Xho I sites, the vaccinia virus H6 promoter and the ampR gene,

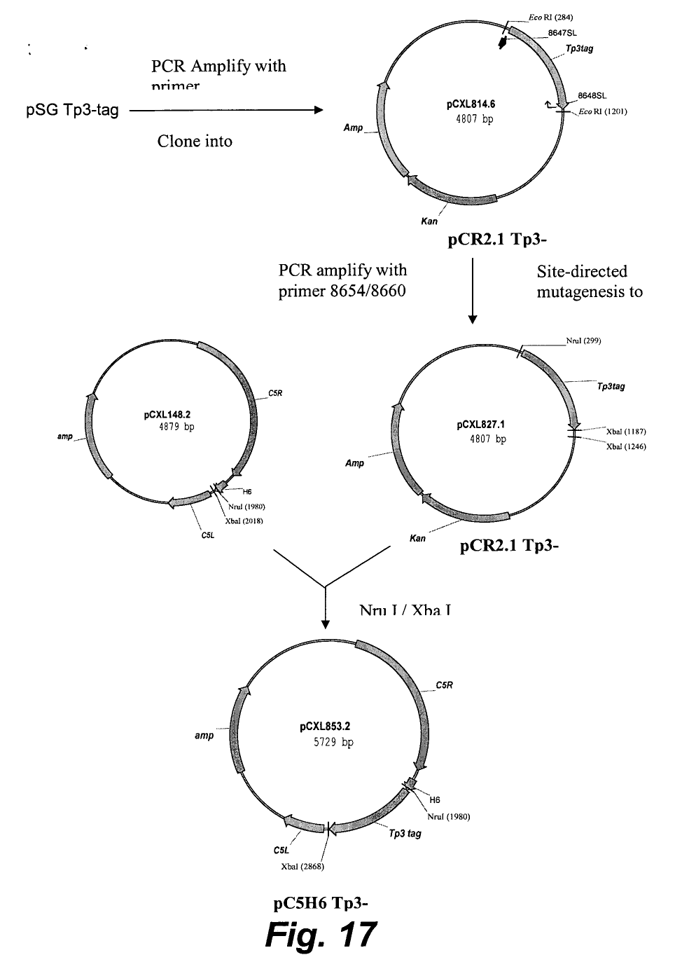

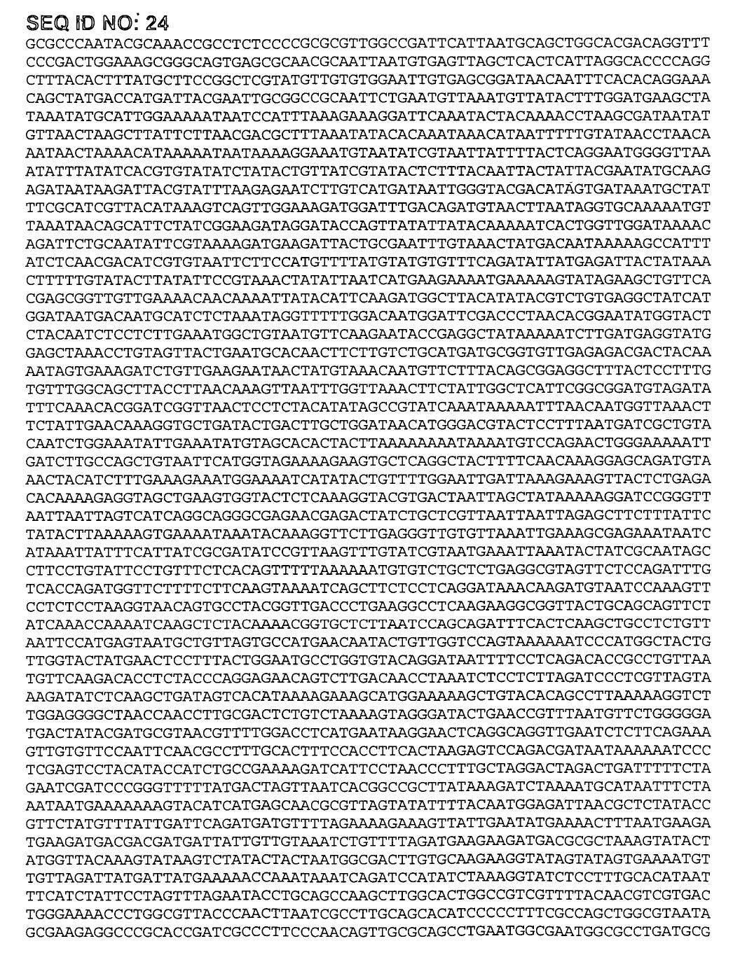

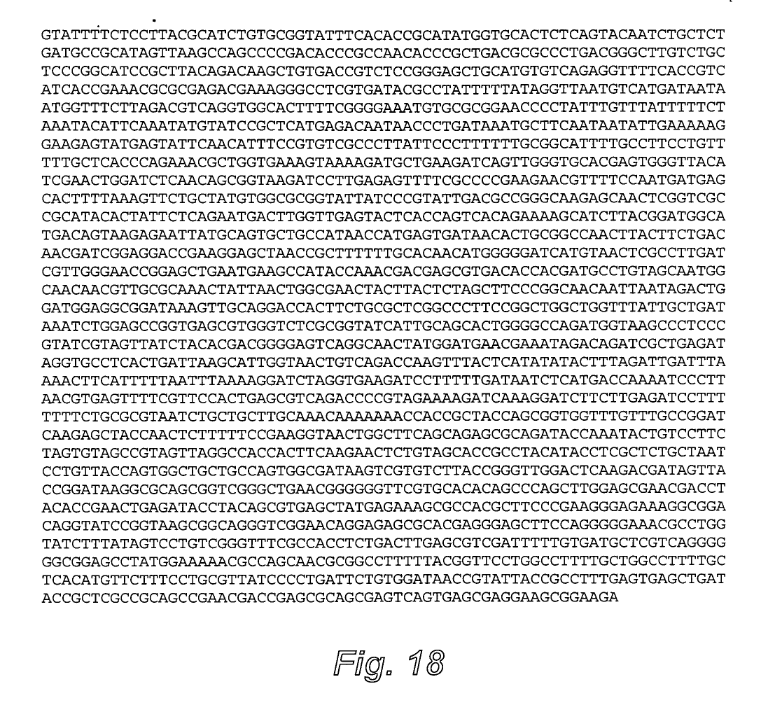

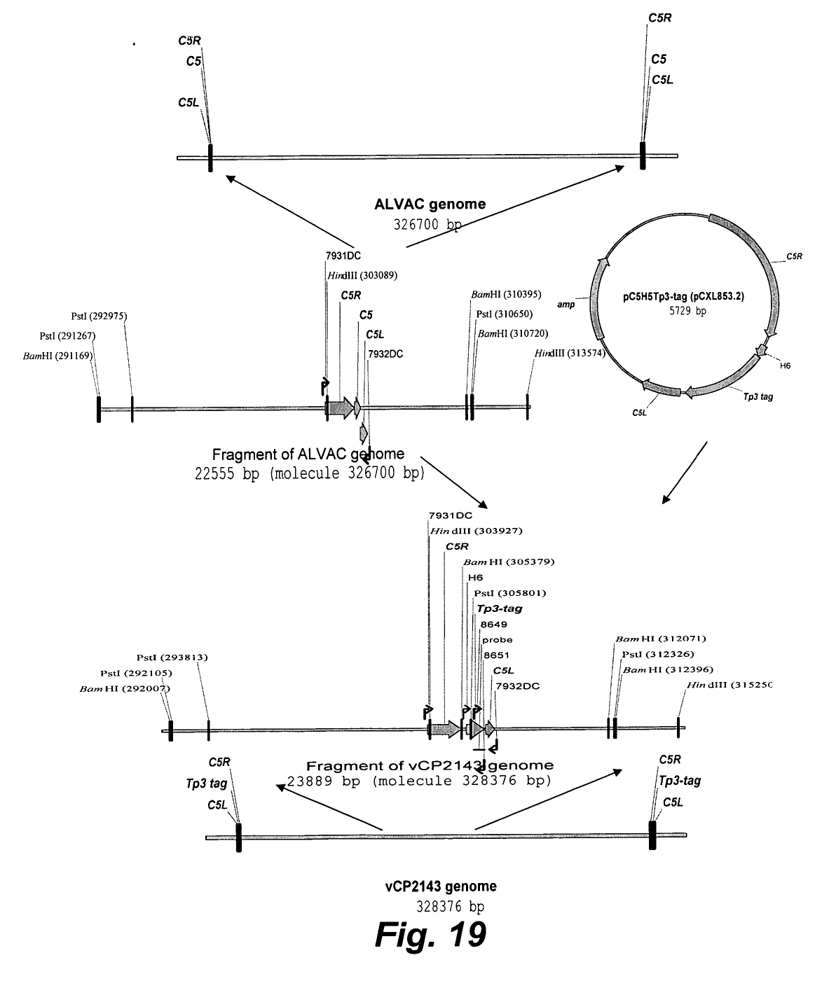

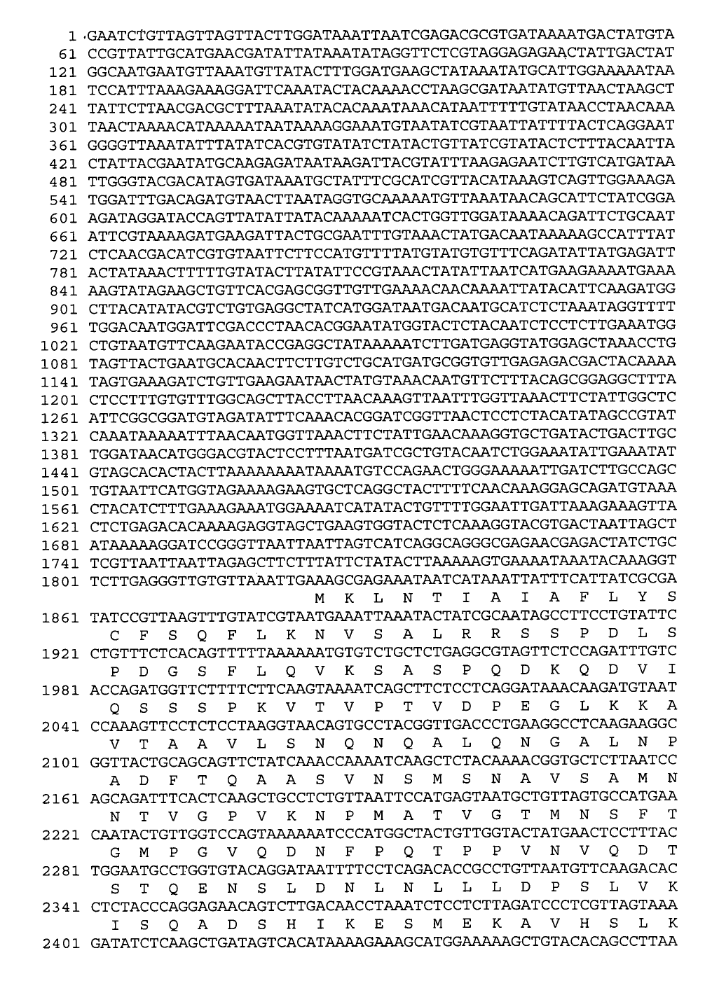

The plasmid construction is schematically illustrated in FIG. 17. PCR amplification was performed using pSG Tp3-tag as template and primers 8648.SL (SEQ ID NO: 20, FIG. 3) and 8647.SL (SEQ IDS NO: 21, FIG. 3). The approximately 800 bp PCR fragment was cloned into pCR2.1, generating plasmid pCXL814.6 (pCR2.1 H6p Tp3-tag). Site-directed mutagenesis using pCXL814.6 as the template and primers 8654.SL: 5′-ATCGCAATAGCCTTCCTGTATTCCTGTTTC-3′ (SEQ ID NO: 22) and 8660.SL: 5′-GAAACAGGAATACAGGAAGGCTATTGCGAT-3′ (SEQ ID NO: 23). The approximately 0.6 kb Nru I-Xba I fragment from pCXL827.1 was inserted into the ALVAC C5 donor plasmid pCXL-148-2 that had been digested with Nru I/Xba I, to generate plasmid pCXL853.2 (pC5H6 Tp3-tag) having the sequence SEQ ID NO: 24 as shown in FIG. 18. Regions within the plasmid pC5H6 Tp3-tag were located at the following nucleotide positions within SEQ ID NO: 24: Amp resistance, 3928-4788; C5R, 248-1783; C5L, 2911-3315; H6, 1881-2004; Tp3 tag, 2005-2859Recombinant generation: The in vivo recombination was performed by transfecting primary CEFs grown as described in Example 1 above, with 9.6 μg of Not I-linearized donor plasmid pCXL853.2 and as schematically illustrated in FIG. 19. The transfected cells were subsequently infected with ALVAC as the rescue virus at a multiplicity of infection (MOI) of 10. After 29 hrs, the transfected-infected cells were harvested, sonicated and used for recombinant virus screening.

Recombinant plaques were screened by plaque lift hybridization method using a 487 bp Tp3 tag specific probe labeled with horseradish peroxidase. After five sequential rounds of plaque purification, the recombinants vCP2143.2.1.1.1.1 and vCP2143.4.1.1.1.1 were obtained and confirmed by hybridization as positive for the Tp3 tag insert and negative for the C5 ORF. Single plaques were selected from fifth round of plaque purification, and expanded to obtain stocks to amplify construct vCP2143.

The infected cell culture fluid from P3 roller bottles was harvested and concentrated to produce virus stock (1.8 ml at 3.4×10¹⁰ pfu/ml and 1.8 ml at 2.×10¹⁰ pfu/ml respectively, for vCP2143.2.1.1.1.1 and vCP2143.4.1.1.1.1.).

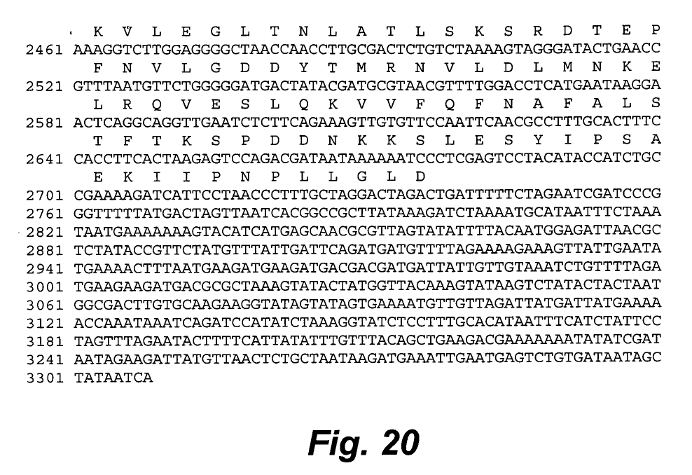

Sequence analysis: Analysis of viral vCP2143 stock genomic DNA was by PCR amplification and sequence analysis of the flanking arms of the C5 locus and the Tp3-tag insert. Primers 7931.DC (SEQ ID NO: 10) and 7932.DC 5′ (SEQ ID NO: 11) were used to amplify the entire C5L-Tp3 TAG-C5R fragment. The nucleotide sequence of ALVAC vCP2143 (SEQ ID NO: 25) is shown in FIG. 20. In vCP2143 (SEQ ID NO: 25), the various regions are at the following nucleotide positions: Tp3 tag, 1883-2737; Probe, 2251-2737; C5R, 126-1661; C5L, 2789-3193; Primer 7931DC, 1-25; Primer 8649SL, 2251-2274; Primer 8651SL, 2714-2737 (on complementary strand); primer 7932DC, 3284-3308 (on complementary strand); promoter H6, 1759-1882. The amino acid sequence SEQ ID NO: 26 of the encoded Tp3 region is shown in FIG. 6.

Expression analysis: Primary CEFs were infected with P3 viral stock at a MOI of 10 and incubated at 37° C. for 27 hrs. The cells and culture supernatant were harvested and extracted sample proteins were separated on a 10% SDS-PAGE gel, transferred to Immobilon nylon membrane, and probed with an anti-V5-TAG-specific mAb (Serotech MCA1360 at 1 in 2,000 dilution). Peroxidase-conjugated goat anti-mouse antiserum was used as secondary antibody and the bands were visualized using luminol reagents.

### EXAMPLE 8

### Construction of ALVAC Vector Encoding Tp4

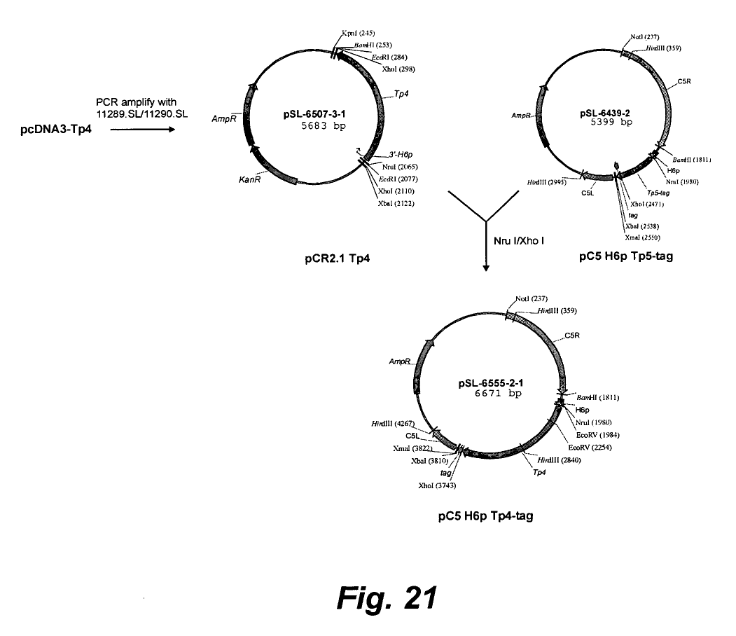

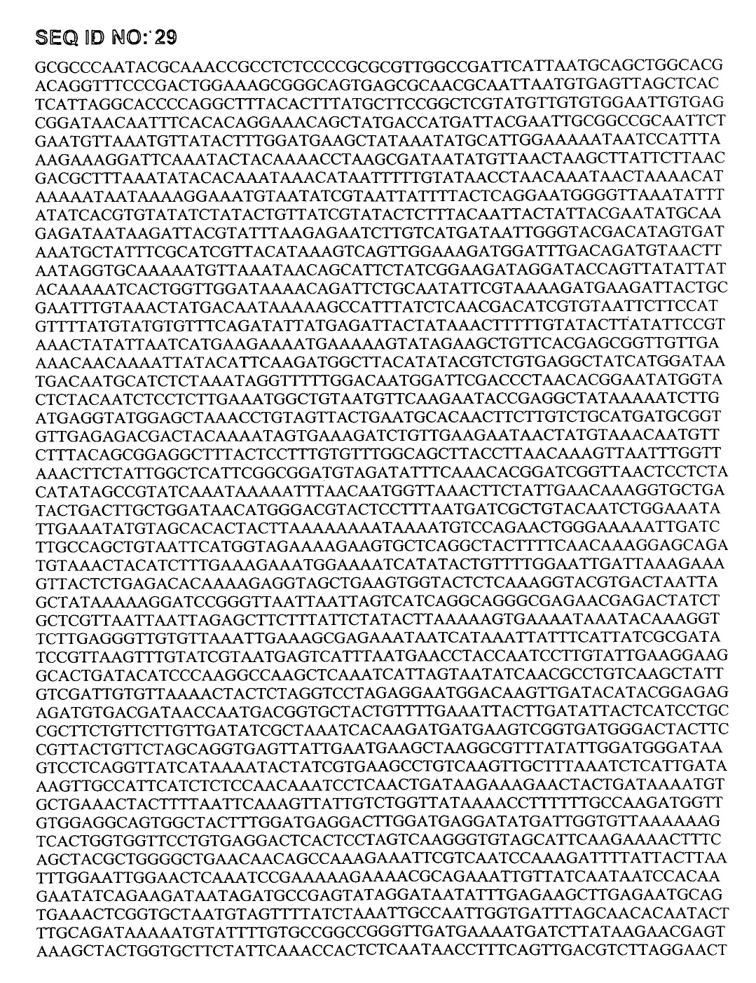

Plasmid construction: Plasmid pcDNA3-Tp4 was used to construct a donor plasmid for insertion of H6p Tp4, with an expression tag, into the C5 loci of ALVAC as shown schematically in FIG. 21. A PCR reaction was performed using pcDNA3-Tp4 as template with primers 11289.SL (SEQ ID NO: 27, FIG. 3) and 11290.SL (SEQ ID NO: 28, FIG. 3). These primers introduced the 3′-end of the H6 promoter and a 3′-Xho I site for linking to the P. berghei tag sequence. The PCR product was cloned into pCR2.1 to generate plasmid pSL-6507-3-1 (pCR2.1 Tp4).

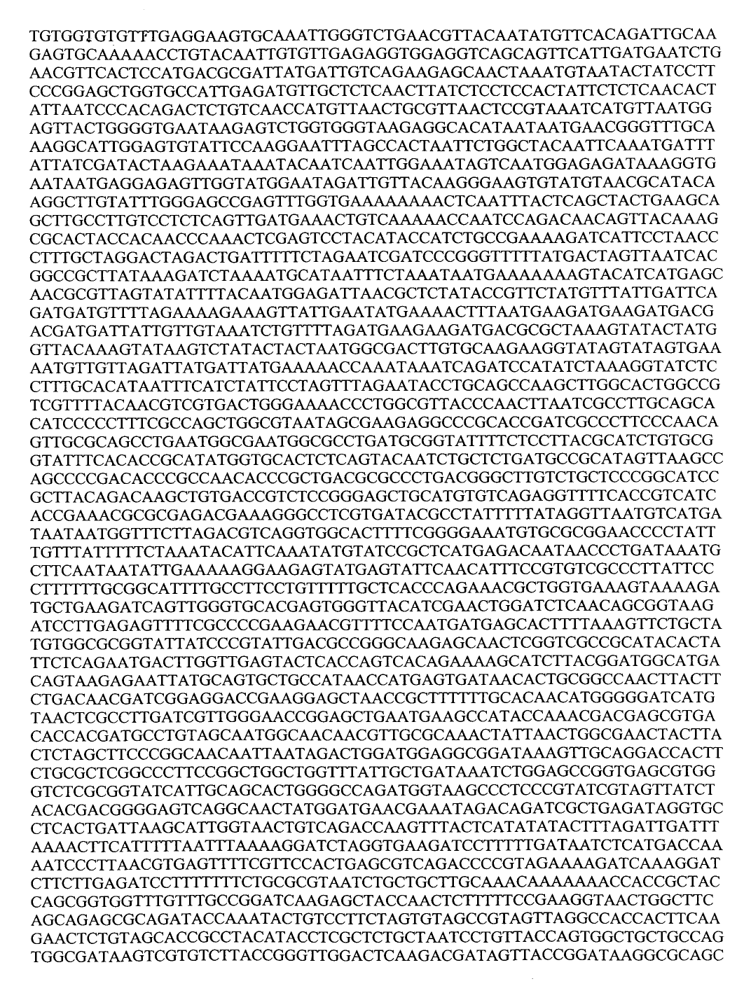

Plasmid pSL-6439-2 (pC5 H6p Tp5-tag) was used as the source of pC5 vector backbone, the 5′-end of H6p and the P. berghei tag sequence. Plasmid pSL-6439-2 was digested with Nru I and Xho I and ligated with the 1.8 kb Nru I/Xho I Tp4 fragment from pSL-6507-3-1, to generate pSL-6555-2-1 (pC5 H6p Tp4-tag), the nucleotide sequence of the insert of which (SEQ ID NO: 29) shown in FIG. 22. Nucleotide positions within pSL-6555-2-1 of various regions are as follows: C5R, 236-1809; H6p, 1881-2004; Tp4, 2005-3741; tag, 3742-3801; C5L, 3854-4263; AmpR, 4870-5727. The amino acid sequence (SEQ ID NO: 30) of the Tp4-tag product is shown in FIG. 6D.

### EXAMPLE 9

### Construction of ALVAC vCP2138 Encoding TP5

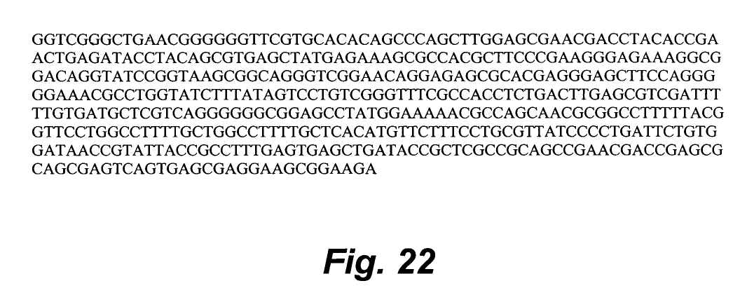

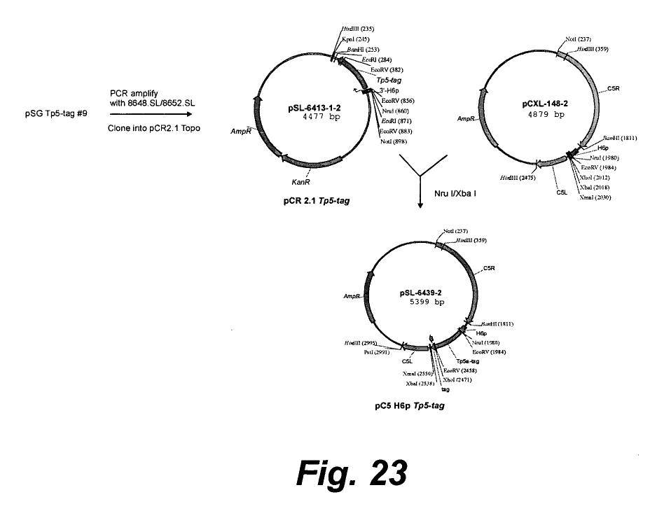

Plasmid construction: Plasmid pSG Tp5-tag #9 encoding: T. parva gene 5 with a marker tag from the circumsporozoite protein of Plasmodium berghei was used to construct a donor plasmid for insertion of Tp5-tag into the C5 loci of ALVAC according to the scheme illustrated in FIG. 23.

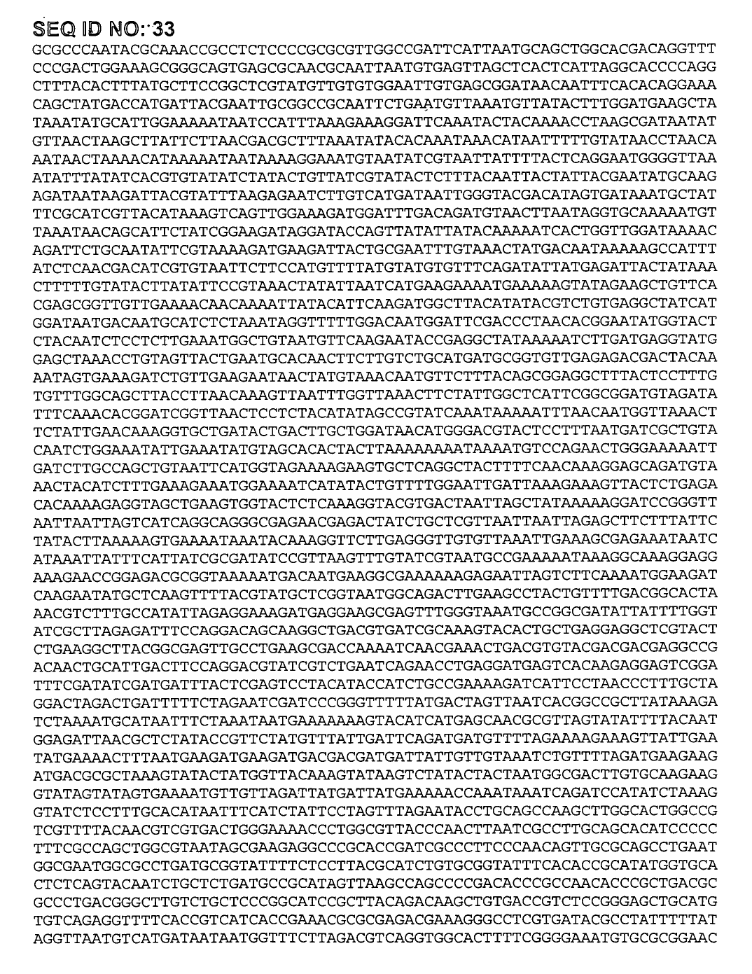

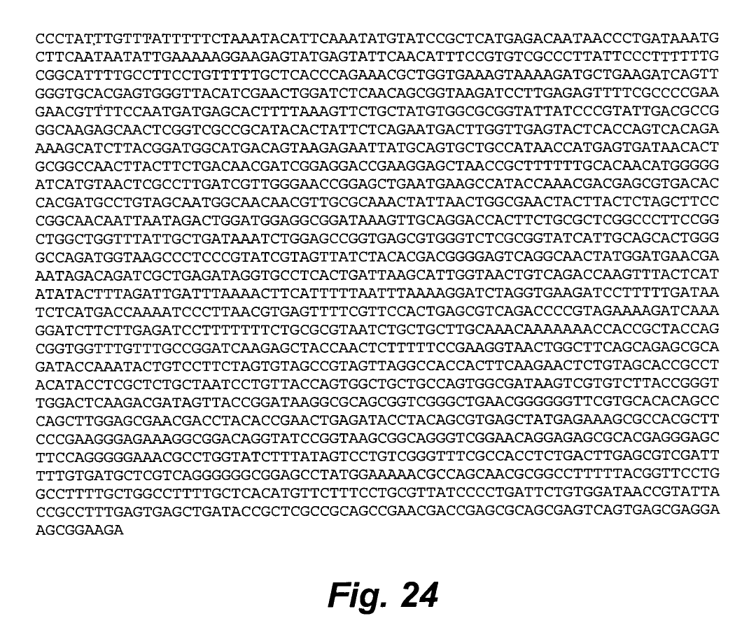

PCR amplification was performed using pSG Tp5-tag as the template and the primers 8648.SL (SEQ ID NO: 20, FIG. 3) and 8652.SL (SEQ ID NO: 31, FIG. 3). The approximately 600 bp PCR fragment was cloned into pCR2.1, generating plasmid pSL-6413-1-2 (pCR2.1 H6p Tp5-tag) which was sequenced. The approximately 0.6 kb Nru I-Xba I fragment from pSL-6413-1-2 was inserted into the ALVAC C5 donor plasmid pCXL-148-2, which had been digested with Nru I/Xba I, to generate plasmid pSL-6439-2. The amino acid sequence of the Tp5-tag product (SEQ ID NO: 32) is illustrated in FIG. 6E. The nucleotide sequence of the insert of construct pSL-6439-2 (SEQ ID NO: 33) is illustrated in FIG. 24.

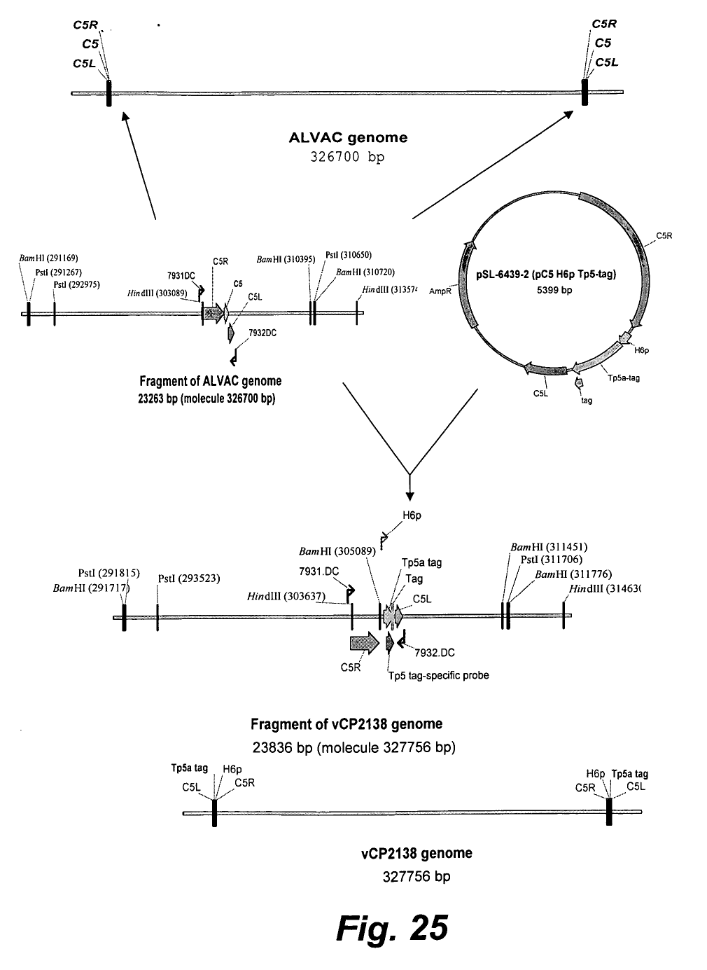

Generation and characterization of Canary pox recombinant containing Tp5-tag inserted in C5 loci of ALVAC (vCP2138): In vivo recombination according to the schematic illustrated in FIG. 25 was performed by transfection of primary CEFs with Not I-linearized donor plasmid pSL-6439-2 (15 μg). The transfected cells were subsequently infected with ALVAC-1 as the rescue virus at an MOI of 10 (Stock date # 1387). After 24 hrs, the transfected-infected cells were harvested, sonicated and used for recombinant virus screening.

Recombinant plaques were screened by plaque lift hybridization method using a Tp5-tag-specific probe (prepared using the primers 8651.SL: 5′-GTCTAGTCCTAGCAAAGGGTTAGG-3′ (SEQ ID NO: 34) and 8653.SL: 5-GCTCGGTAATGGCAGACTTGAAGC-3′ (SEQ ID NO: 35) labeled with horseradish peroxidase. After 6 and 7 sequential rounds of plaque purification, the recombinants vCP2138.16.2.2.1.2.4.6 and vCP2138.19.2.2.1.1.1 were isolated and confirmed by hybridization as positive for the Tp5-tag insert and negative for the C5 ORF.

Single plaques were selected from the sixth and seventh rounds of plaque purification, were expanded to obtain P1, P2, and P3 (roller bottle) stocks to amplify vCP2138. The infected cell culture fluid from the roller bottles was harvested and concentrated to produce virus stock (2.1 ml of vCP2138.19.2.2.1.1.1 at 2.7×10¹⁰ pfu/mL and 2.1 ml of vCP2138.16.2.2.1.2.4.6 at 2.2×10¹⁰ pfu/mL).

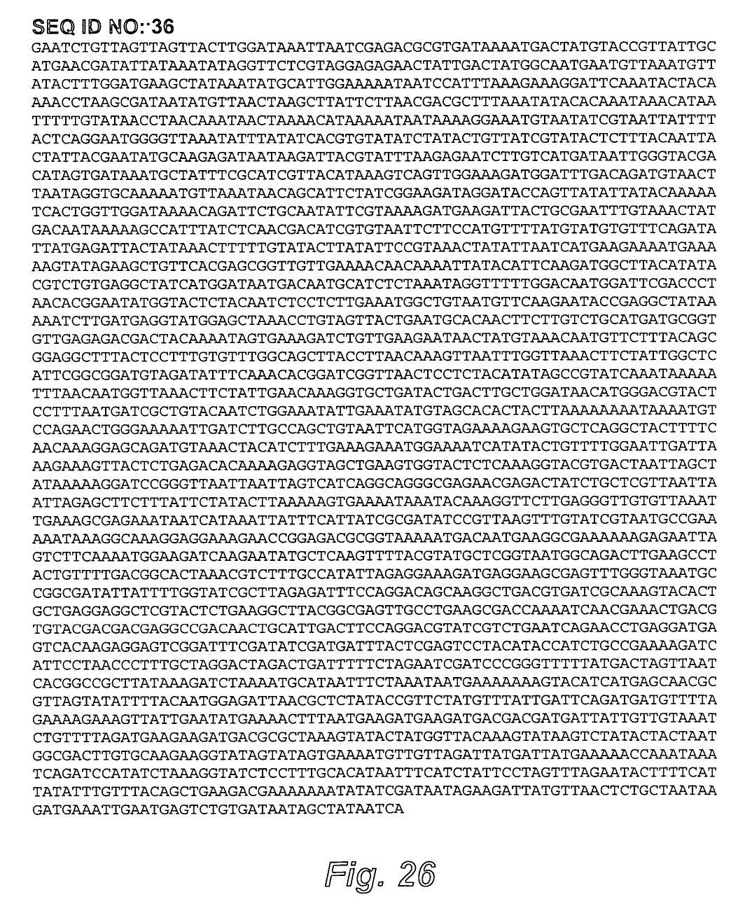

Sequence analysis: A more detailed analysis of the P3 viral stock genomic DNA was performed by PCR amplification and sequence analysis of the flanking arms of the C5 locus and the Tp5-tag insert. Primers 7931.DC (SEQ ID NO: 10) and 7932.DC (SEQ ID NO: 11) were used to amplify the entire C5R-H6p Tp5-tag-C5L insert fragment, the nucleotide sequence (SEQ ID NO: 36) of which is shown in FIG. 26. The nucleotide positions of the various domains within sequence SEQ ID NO: 36 are as follows: Tp5-tag, 1883-2407; Tp5-specific probe, 2005-2407; C5R, 126-1661; C5L, 2459-2863; H6 promoter, 1759-1882. The vector vCP2138, therefore, comprised the 4016 bp insert SEQ ID NO: 36 and the ALVAC sequences for a total length of 327756 bp.

### EXAMPLE 10

### Construction of pC5 H6p Tp6-tag (pHM1103.1) Donor Plasmid

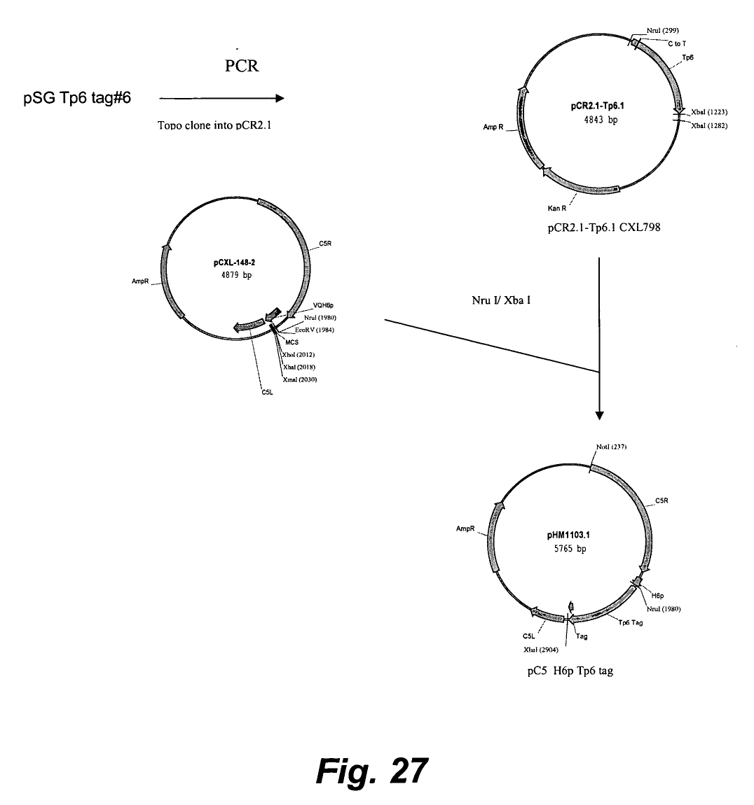

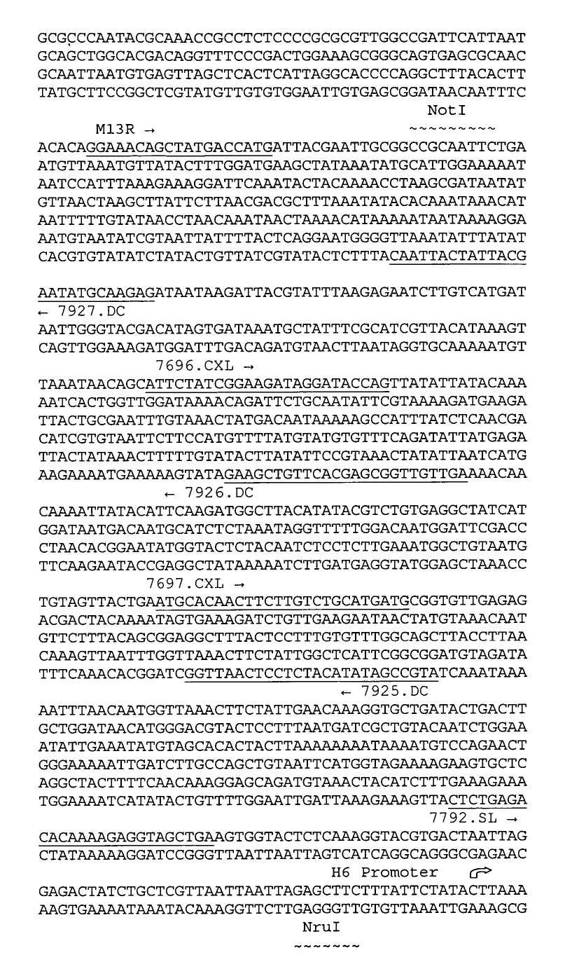

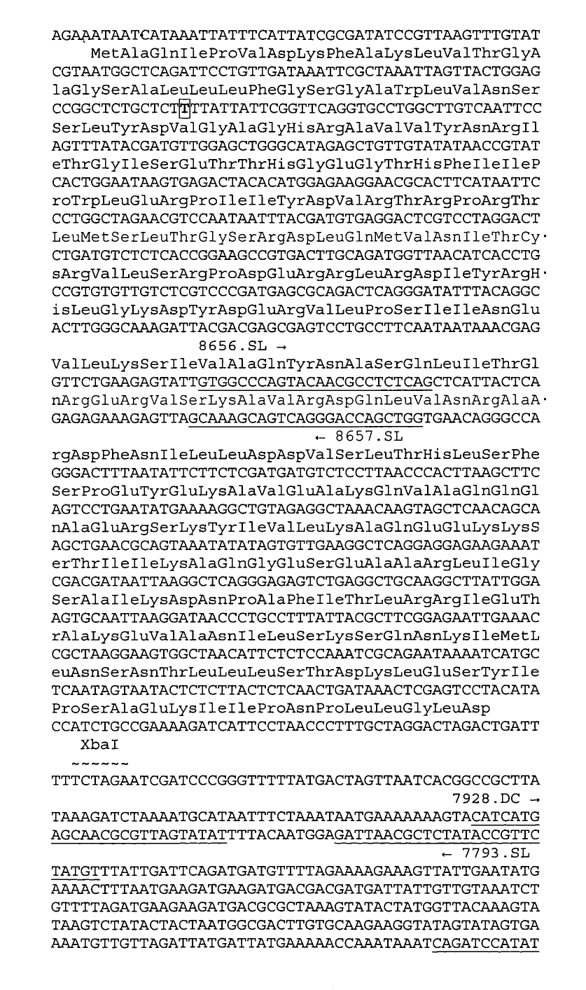

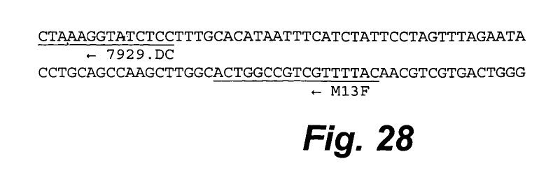

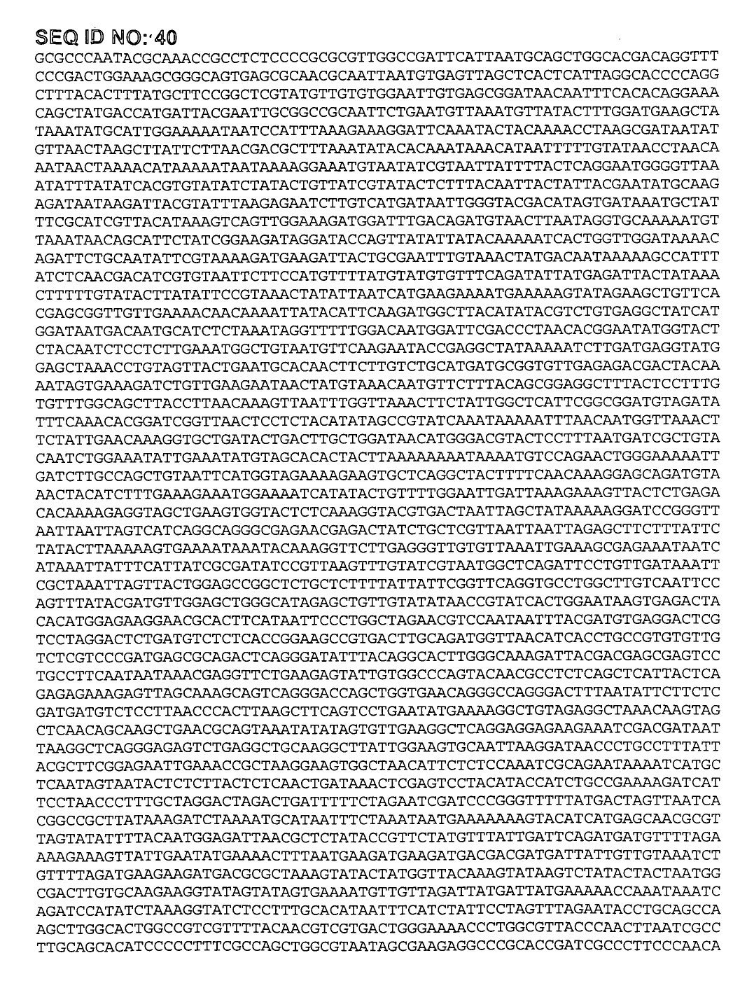

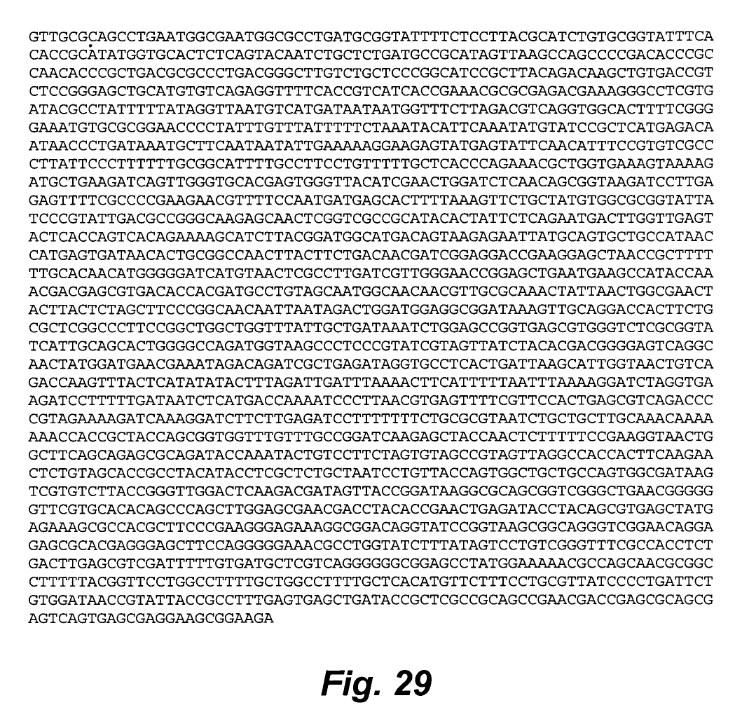

Plasmid construction: The Tp6-tag gene was amplified from the pSG Tp6 tag#6 template using primers 8648.SL (SEQ ID NO: 20, FIG. 3) and 8655.SL (SEQ ID NO: 37, FIG. 3), which introduce the 3′-end of the H6 promoter and the T5NT poxvirus transcription termination signals. As shown in the schematic illustrated in FIG. 27, using the TA TOPO® cloning system (Invitrogen, Inc), the PCR product was cloned into pCR 2.1-TOPO. Clone pCR2.1 Tp6.1 (CXL798) was confirmed by sequence analysis except for a single base pair change of a C to a T at base pair 2064 resulting in a silent mutation at amino acid 20 (leucine). Plasmid pCR2.1 Tp6.1 was digested with Nru I and Xba I and the 0.9 Kb fragment containing the 3′ end of the H6p and the Tp6-tag gene was inserted into pCXL-148-2 that had been digested with Nru I and Xba I to generate pHM1103.1 (pC5 H6p Tp6-tag). Regions within the insert nucleotide sequence SEQ ID NO: 38 of the pHM1103 plasmid, illustrated in FIG. 28, are: Tp6 Tag, 2005-2895; Tag, 2836-2895; C5R, 236-1809; C5L, 2948-3357; H6p, 1881-2004. The amino acid sequence (SEQ ID NO: 39) of the Tp6 product encoded by this plasmid is shown in FIG. 6F. The nucleotide sequence of the entire pHM1103 plasmid (SEQ ID NO: 40) is illustrated in FIG. 29.

### EXAMPLE 11

### Generation and Characterization of Canary Pox Recombinant Encoding Tp8-tag Inserted in C5 Loci of ALVAC (vCP2179).

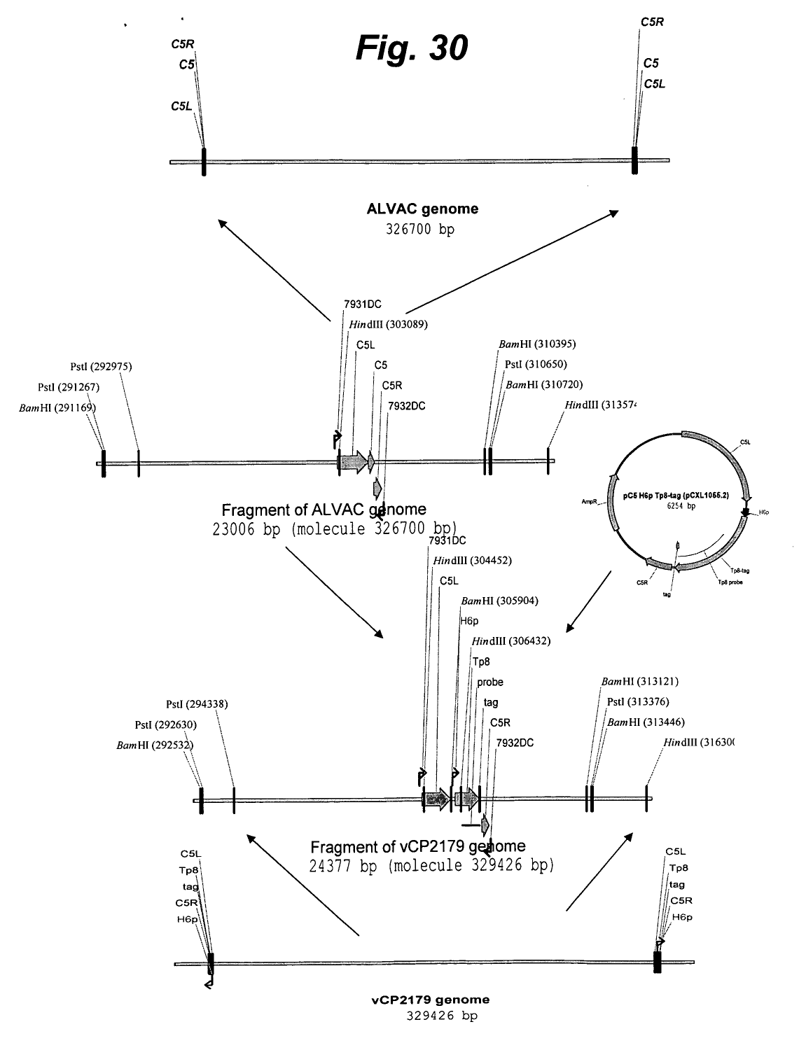

Recombinant generation: In vivo recombination, according to the schematic shown in FIG. 30, was performed by transfecting primary CEFs with 20 μg of Not I-linearized donor plasmid pCXL1055.2 having the Tp8 tag insert using FuGENE-6® reagent (Roche). The transfected cells were subsequently infected with ALVAC as rescue virus at an MOI of 10. After 30.5 hrs, the transfected-infected cells were harvested, sonicated and used for recombinant virus screening.

Recombinant plaques were screened by plaque lift hybridization method using a 923 bp Tp8-tag-specific probe amplified using the primers 11360SL: 5′-GTTCTTTACAAGGTTGCAGTCGAG-3′ (SEQ ID NO: 41) and 11362SL: 5′-GACTCGAGAACTGCGTACACTGCGAAGGAGGT 3′ (SEQ ID NO: 42) labeled with horse radish peroxidase. After four sequential rounds of plaque purification, recombinants vCP2179.1.1.1.2 and vCP2179.2.1.1.1 were isolated and confirmed by hybridization as positive for the Tp8-tag insert and negative for the C5 ORF.

Single plaques were selected from fourth round of plaque purification, and expanded to obtain P1, P2 and P3 (4× roller bottles per sister construct in the first batch, then 8 roller bottles for vCP2179.2.1.1.1 in the second batch) stocks to amplify construct vCP2179. The infected cell culture fluid from the roller bottles was harvested and concentrated to produce virus stock. Titers of the concentrated stocks were: (first batch) vCP2179.1.1.1.2, 2.7×10⁹ pfu/ml in 2.7 ml; vCP2179.2.1.1.1, 5.4×10⁸ pfu/ml in 2.7 ml; (second batch) vCP2179.2.1.1.1, 3.6×10⁹ pfu/ml in 5.0 ml.

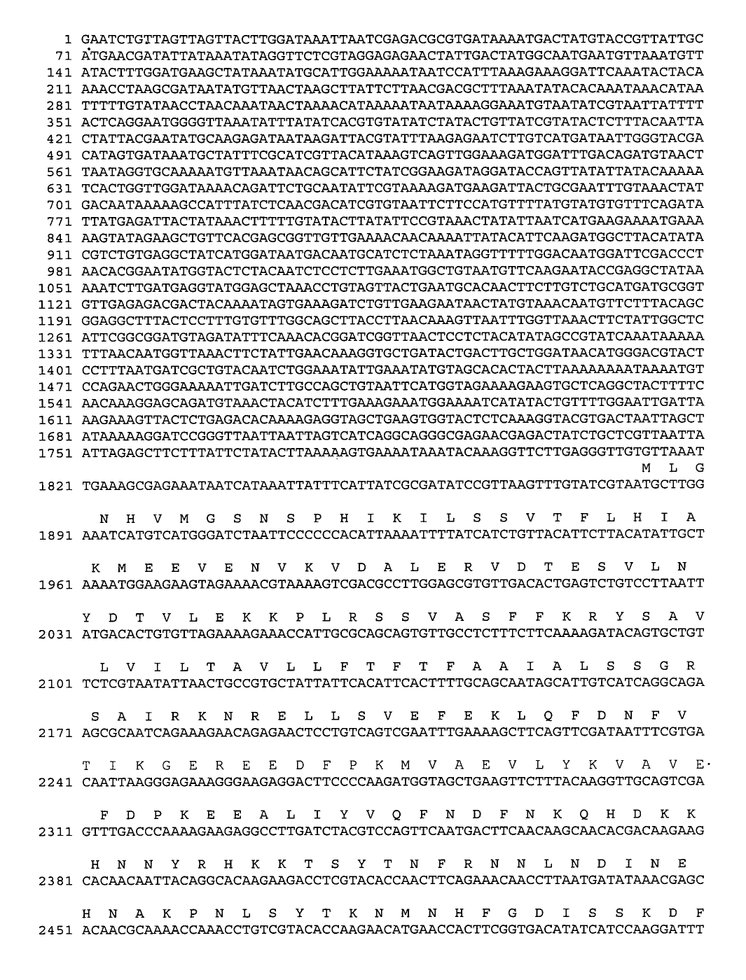

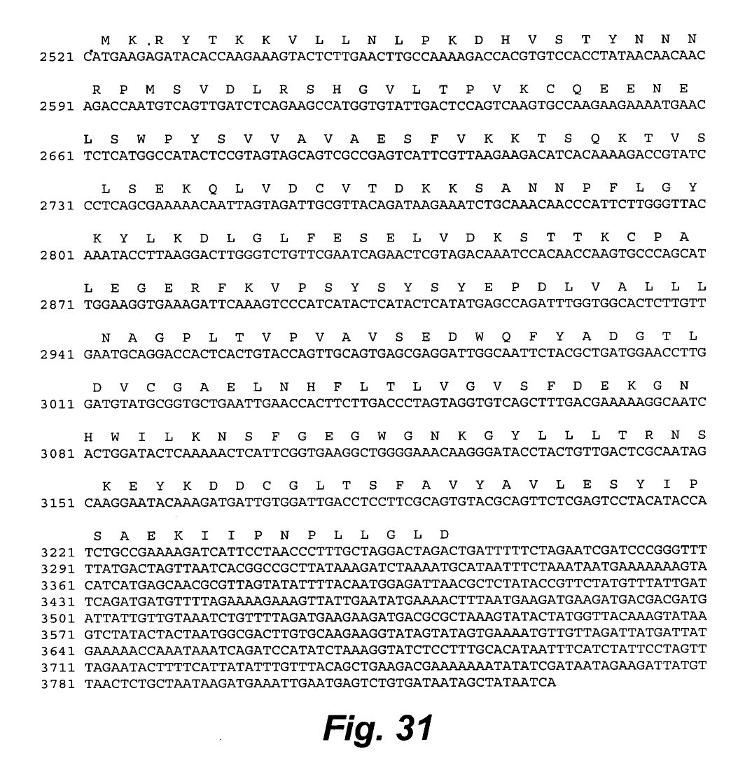

Sequence analysis: Analysis of the P3 stock genomic DNA was performed by PCR amplification and sequence analysis of the flanking arms of the C5 locus and the Tp8-tag insert. Primers 7931DC (SEQ ID NO: 10 and 7932DC (SEQ ID NO: 11) located beyond the arms of the C5 locus and within the ALVAC genome were used to amplify the entire C5L-Tp8-tag-C5R insert fragment, which was then sequenced. The nucleotide sequence SEQ ID NO: 43 of the amplified insert is shown in FIG. 31. Within SEQ ID NO: 43 the various regions are located at nucleotide positions as follows: Tp8, 1883-3202; Tag, 3203-3262; Tp8-specific probe, 2288-3210; C5L, 126-1661; C5R, 3314-3718; Primer 7931DC, 1-25; Primer 11360, 2288-2311; Primer 11362, 3179-3210 (on complementary strand); Primer 7932DC, 3809-3833 (on complementary strand); promoter H6p, 1759-1882. The amino acid sequence SEQ ID NO: 44 of the Tp8 polypeptide encoded by the cloned insert is shown in FIG. 6G.

### EXAMPLE 12

### Vaccine Construct Expression Data

The expression vector constructs vCP2179, ALVAC-Tp8-tag and vFP2201 TROVAC-Tp2-tag were expressed in primary chicken embryo fibroblasts (CEFs).

FIG. 18 illustrates an immunoblot analysis of the products expressed from vCP2179, ALVAC-Tp8-tag. FIG. 19 illustrates an immunoblot analysis of the products expressed from vFP2201 TROVAC-Tp2-tag and a comparison with the products expressed from vCP2143 ALVAC-Tp3-tag.

### EXAMPLE 13

### Infection and Treatment Immunization of Cattle and CTL

Fourteen cattle, 3 Boran (Bos indicus), 2 Jersey (Bos taurus), 2 Friesian (do.) and 7 crossbreds, were immunized by the infection and treatment method against the Muguga stock of T. parva by simultaneous inoculation of sporozoites (1:20 dilution of sporozoite stabilate # 4133, ILRI, Nairobi, Kenya) and long-acting oxytetracycline at 20 mg/kg body weight as described by Taracha et al., (1996) Infect. Immun. 63: 1258. Cattle were challenged with 1:20 dilution of sporozoite stabilate several months after immunization. Following immunization or challenge, animals were monitored for clinical and parasitological changes. Giemsa-stained biopsy smears were examined for the presence of schizont-infected cells and scored on a scale of 0-3. Prior to immunization, venous blood was collected from animals, peripheral blood mononuclearcytes (PBMCs), purified and infected in vitro with T. parva (Muguga) sporozoites as described by Goddeeris & Morrison, (1988) J. T. Cult. Methods. 11: 101. Infected lymphoblasts were maintained in RPMI-1640 supplemented with 10% Fetalclone II (Perbio Science UK, Ltd. Cramlington, UK; tested for BVDV & Mycoplasma spp.), 100 iU/ml penicillin, 100 μg/ml streptomycin, 50 μg/ml gentamycin, 5×10⁻⁵M 2-mercaptoethanol and 2mM L-glutamine and passaged 1:5 three times a week as established T. parva-infected lymphoblasts. Schizont-specific polyclonal CTL lines and clones were established from PBMC obtained from immunized animals by repeated restimulation with autologous infected lymphoblasts, maintained and assessed for cytotoxic activity as previously described by Goddeeris & Morrison, (1988) J. T. Cult. Methods. 11: 101.

### EXAMPLE 14

### Generation, Immortalization and Transfection of Bovine Skin Fibroblasts

Skin biopsies were surgically collected and skin fibroblasts (SFs) prepared using standard protocols. Skin fibroblasts (SF) were maintained in DMEM containing 10% FBS (Perbio Science), 100 iU/ml penicillin, 100 μg/ml streptomycin and 2 mM L-glutamine and immortalized by the stable integration of the gene encoding the SV40 large T antigen. Sub-confluent SF monolayers were transfected with pSV3-neo (ATCC code 37150, ATCC, Rockville MD, USA) using Fugene 6 transfection reagent as described by the manufacturer (Roche Diagnostics GmbH, Mannheim, Germany). Two hrs post-transfection, medium was removed and replaced with DMEM containing 10% FCS and 0.5 μg/ml of geneticin (Sigma) and incubated further. Cells were maintained under selection, positive colonies were expanded and expression of SV40 large T-antigen was assessed using immunoperoxidase staining (SV40 T Ag (Ab-2), Oncogene Research Products, San Diego, Calif., USA). Further expansion and maintenance of SFs was carried out using complete DMEM. Immortalized SFs (iSFs) were dispensed, 2×10⁵/well, 96 well flat-bottom plates, incubated overnight and transfected with plasmids expressing targeted genes or schizont cDNA pools (100 ng/well) using Fugene 6 transfection reagent. COS-7 cells were co-transfected with 100 ng/well of each targeted gene or schizont cDNA pool together with 50 ng/well BoLA cDNA.

### EXAMPLE 15

### Detection of CTL Recognition of Transfected iSF by IFN-γ ELISpot

Twenty-four hours post-transfection, transfected iSF were washed with PBS (200 μl/well), detached with 100 μl/well 0.25% Trypsin-EDTA and transferred to 96 well round-bottom plates (Costar) containing 100 μl/well cold RPMI supplemented with 10% FCS. Cells were centrifuged at 1200 rpm for 3 min, supernatant removed and cells resuspended in 100 μl of RPMI-1640 supplemented as described above. Schizont-specific CTL, generated and maintained as described above, were harvested 7-14 days post-stimulation, and resuspended at 2×10⁵/ml in RPMI-1640 medium supplemented with 10% FBS and 5 U/ml recombinant human IL-2 (Sigma). ELISpot plates (Millipore Corporation, Bedford, Mass., USA) were coated with 50 μl/well of 1 μg/ml of murine anti-bovine IFN-γ monoclonal antibody (CC302; Serotec, Oxford, UK) and incubated overnight at 4° Celsius. Wells were washed twice with unsupplemented RPMI-1640 and blocked using 200 μl/well with RPMI-1640 supplemented with 10% FBS by incubating at 37° Celsius for 2 hours. The blocking medium was removed, replaced with 50 μl/well CTL and 100 μl/well transfected cells, and incubated in a humidified incubator at 37° Celsius for 20 hrs. After incubation, the contents of wells were removed and wells washed four times with distilled water containing 0.05% Tween 20 with the plate shaken for 30 secs between washes. The process was repeated an additional four times, using PBS containing 0.05% Tween 20. Plates were then developed for IFN-γ spots and read as described in Taracha et al., (2003) Infect. Immun. 71: 6906

### EXAMPLE 16

### Detection of CTL Lysis of Transfected-iSF

Autologous iSFs were seeded at 2.5×10⁵ cells/well in 6-well plates (Costar) and incubated for 2 hours at 37° Celsius to allow cells to adhere. Cells were transfected using Fugene 6 transfection reagent as described by the manufacturer. Twenty-four hours post-transfection, cells were harvested as described above. Transfected cells and schizont-infected cells were labeled with ⁵¹Chromium (Amersham Biosciences Europe GmbH, Freiburg, Germany) and the ability of schizont-specific CTL lines (day 6-8 post-stimulation) to lyse these targets was assessed as described in Example 15

### EXAMPLE 17

### Ex vivo Detection of Antigen-Specifilc CD8⁺ T Cell Responses

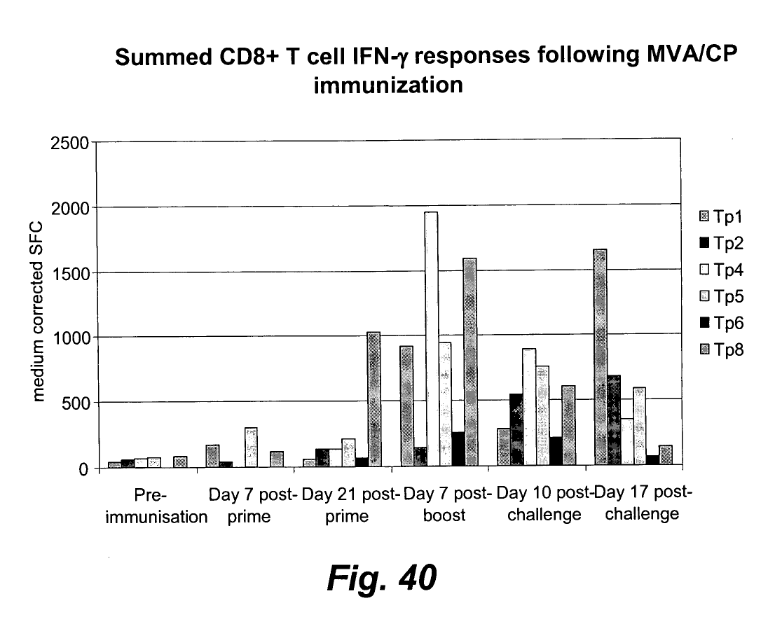

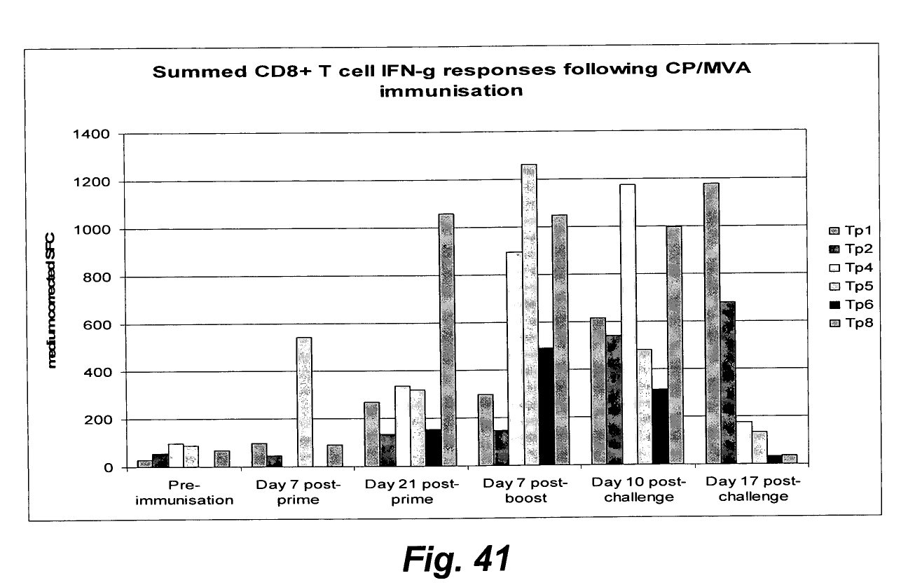

Cattle donors for schizont-specific CTL used to identify Tp1-8, were challenged with a lethal dose of T. parva (Muguga) sporozoites and bled on selected days post-challenge. CD8⁺ T cells and CD14⁺ monocytes were purified from PBMC by MACS magnetic cell sorting according to the manufacturer's instructions (Miltenyi Biotec, Gergisch Gladbach, Germany). CD8⁺ T cells were sorted indirectly using a monoclonal antibody specific for bovine CD8 (IL-N*001015; ILRI, Nairobi, Kenya) followed by incubation with goat anti-mouse IgG microbeads (Miltenyi Biotec). CD14⁺ monocytes were sorted directly by incubation with CD14 microbeads (Miltenyi Biotec). CD8⁺ T cells (2.5×10⁵ /well) and CD14⁺ monocytes (2.5×10⁴/well) were added to IFN-γ ELISpot plates containing synthetic peptides (1 μg/ml final concentration) and were incubated and developed as described above.
FIG. 40 illustrates summed CD8+ T cell IFN-g responses following MVA/CP immunization.
FIG. 41 illustrates summed CD8+ T cell IFN-g responses following CP/MVA immunization.

### EXAMPLE 18

### Theileria parva Vaccination/Challenge

Twenty-eight Boran (Bos indicus), Friesian (Bos taurus), and crossbred calves, 4-24 months old and expressing one of four MHC class I alleles were randomized into three groups as shown in Table 4.

TABLE 4
Cattle and their responses to heterologous prime-boost immunization with CTL target
antigens and challenge with T. parva.

| | | | | | | CD8⁺ | | |
|---|---|---|---|---|---|---|---|---|
| | | | | | BoLA | T cell | | ECF |
| Group | Animal No | Breed | Sex | Age (mo) | allele | IFN-γ | CTL | Index |
| CP/ | BX215 | Boran | Female | 22.33 | N*00101 | + | − | 4.63 |
| MVA | BZ027 | Friesian | Male | 8.26 | N*01301 | + | + | 6.31 |
| | BZ017 | Friesian | Male | 6.36 | T5* | + | + | 7.26 |
| | BY142 | Crossbred | Female | 8.69 | T2a | − | − | 7.42 |
| | BY206 | Crossbred | Female | 13.51 | T2a | + | + | 7.45 |
| | BZ039 | Friesian | Female | 15.67 | N*01301 | + | + | 7.85 |
| | BX223 | Boran | Male | 24.89 | N*00101 | + | − | 7.97 |
| | BX216 | Boran | Male | 21.93 | N*00101 | + | − | 8.45 |
| | BZ026 | Friesian | Male | 9.74 | T2a | − | − | 8.64 |
| | BZ016 | Friesian | Male | 6.36 | T5 | + | − | 8.70 |
| | BZ018 | Friesian | Male | 4.33 | N*01301 | + | − | 8.93 |
| | BZ022 | Friesian | Male | 10.03 | T5 | + | − | 8.93 |
| DNA/ | BY001 | Crossbred | Male | 15.93 | T2a | + | + | 3.39 |
| MVA | BX219 | Boran | Male | 24.69 | N*00101 | + | + | 6.28 |
| | BZ023 | Friesian | Male | 7.02 | N*01301 | + | + | 6.96 |
| | BY138 | Friesian | Male | 20.45 | T2a | − | − | 7.09 |
| | BZ013 | Friesian | Male | 18.39 | T5 | + | − | 7.28 |
| | BX225 | Boran | Male | 21.87 | N*00101 | + | − | 7.61 |
| | BX220 | Boran | Female | 22.23 | N*00101 | − | − | 8.44 |
| | BZ019 | Friesian | Male | 4.20 | T5 | + | − | 8.59 |
| | BZ020 | Friesian | Male | 12.07 | T2a | − | − | 8.60 |
| | BY100 | Crossbred | Male | 10.92 | N*01301 | + | − | 8.78 |
| | BZ025 | Friesian | Male | 9.64 | T5 | + | − | 8.83 |
| | BZ040 | Friesian | Female | 7.34 | N*01301 | + | − | 8.91 |
| Control | BY029 | Crossbred | Male | 13.05 | T2a | − | − | 7.88 |
| | BY071 | Friesian | Male | 26.82 | T5 | − | − | 8.50 |
| | BZ038 | Friesian | Female | 16.69 | N*01301 | − | − | 8.76 |
| | BX218 | Boran | Female | 24.82 | N*00101 | − | − | 8.90 |

Ages of cattle at the time of priming immunization are shown. Cattle were defined as CD8⁺ T cell IFN-γ and CTL responders (+) or non-responders (−). DPC , Days post-challenge

Recombinant DNA, CP and MVA constructs expressing single antigens (Tp1, Tp2, Tp4, Tp5 and Tp8) were prepared separately and inoculated at different sites. Cattle were primed with 0.5 mg of each recombinant DNA plasmid by intradermal injection or 1×10⁸ pfu of each recombinant canarypox virus by subcutaneous injection given at different sites. After four weeks, cattle were inoculated with 5×10⁸ pfu of each recombinant MVA construct by subcutaneous injection. Three weeks post-boost, cattle were challenged with a lethal dose of T parva (1:20 dilution of T. parva Muguga stock sporozoite stabilate # 4133, ILRI, Nairobi, Kenya) and the response to challenge monitored as described above. Reactions to challenge were calculated as an ECF reaction index by combination of thirteen parameters, including temperature, hematological and parasitological measurements, using a first principle component analysis as described in Rowlands et al., Parasitol. (2000) 120: 371.

Cattle were assessed daily by an independent veterinarian and animals displaying clinical signs of severe ECF were euthanized and the experiment was terminated 21 days post-challenge. Antigen-specific CD4⁺ and CD8⁺ T cell IFN-γ responses were measured by ELISpot assay every 2 weeks over the course of immunization and challenge. CD4⁺ T cells were MACs sorted indirectly using a monoclonal antibody specific for bovine CD4 (IL-A12; ILRI, Nairobi, Kenya), CD8⁺ T cells and CD14⁺ monocytes were sorted as described above and ELISpot assays conducted as described above. Antigen-specific CTL responses were recalled by subjecting PBMC to three rounds of restimulation with T. parva infected cells and cytotoxicity against infected cells and uninfected cells pulsed with antigenic peptides were assayed as described above.

Nineteen (93%) of the vaccinated cattle exhibited antigen specific CD8⁺ T cell IFN-γ responses as shown in Table 4 and were boosted in all but four animals following sporozoite challenge. CD4⁺ T cell IFN-γ responses were weak and detected in only nine (38%) of the twenty-four vaccinated cattle and there was no evidence of boosting upon sporozoite challenge. PBMCs from the twenty four vaccinated cattle were subjected to three rounds of stimulation with autologous schizont-infected cells and their lytic activity assessed on autologous infected cells and peptide-pulsed lymphoblasts. Prior to challenge, four cattle exhibited lytic activity. At two week post-challenge, the same four cattle and an additional three were positive in the assays. The antigen specificity of the lytic response in the cattle paralleled the specificity of their IFN-γ responses.

The significance of the lytic CD8⁺ T cell responses became apparent when they were correlated with the outcome to challenge as shown in Table 5 below.

TABLE 5
Association between antigen-specific lytic CTL
responses and outcome to challenge.

| | Vaccinated cattle | | Control cattle |
|---|---|---|---|
| Outcome to | CTL responder | CTL non-responder | CTL non-responder |
| challenge | (n = 7) | (n = 17) | (n = 4) |
| Survived | 7*** | 3 | 1 |
| Euthanized | 0 | 14 | 3 |
| ECF Index | 6.04*** | 8.04 | 8.49 |

***Survival and ECF index of lytic CTL responder cattle were significantly different from non-responders and control cattle (p < 0.01). CTL responses were not detected in the challenge control group.

All of the challenge controls developed severe ECF, with three animals requiring euthanasia. Of the nineteen cattle that made CD8⁺ T cell IFN-γ responses, nine survived, whereas, all seven cattle that made lytic CD8⁺ T cell responses survived. There was a highly significant association between the ability to mount a lytic response and survival (p<0.001). Most of these cattle suffered moderate to severe ECF as judged by their ECF reaction index, a statistical measurement of disease severity based on clinical and parasitological parameters. Their mean ECF reaction index, however, was lower than that of non-responder and control cattle (p<0.001). Our data suggest that vaccination induced CD8⁺ responses contributed to reducing the severity of disease.

Analysis of variance (ANOVA) was used for the analysis of fixed effects on different traits using SAS Release 8.2 (SAS Institute Inc., Cary, USA). A Chi-square test was used to compare the differences in survival for CTL responders and non-responders.

### EXAMPLE 19

### Survival of Vaccinated Cattle

DNA vaccination according to the protocol shown in Table 6 below was performed intramuscularly into subject cattle used 500 μg of each plasmid construct. The amounts of each construct per 500 ml were: ALVAC constructs, 10⁸ pfu; MVA constructs, 5.10⁸ pfu.

TABLE 6

| | | | Challenge |
|---|---|---|---|
| Group | V1 9Day 0) | V2 (Day 28) | (Day 42) |
| A | ALVAC constructs | MVA constructs | Theileria parva |
| | (Tp1, Tp2, Tp4, Tp5, Tp8) | (Tp1, Tp2, Tp3, | |
| | | Tp5, Tp8) | |
| B | DNA Vaccine constructs | MVA constructs | |
| | (Tp1, Tp2, Tp4, Tp5, Tp8) | (Tp1, Tp2, Tp3, | |
| | | Tp5, Tp8) | |
| C | No Vaccine | No Vaccine | |

The challenge was severe (LD 100). A significant portion of vaccinated cattle survived, and this is the first time such an observation is made after a LD100 challenge.

TABLE 7
Categories of East Coast Fever (ECF) reaction to challenge by delivery systems

| Delivery | | MR/MODR | MODR/SR | MODR/SR | SR | Total | |
|---|---|---|---|---|---|---|---|
| System | No. | (recovered) | (recovered) | (euthanized) | (euthanised) | Protected | Survived |
| vCP/MVA | 12 | 1 | 5 | 1 | 5 | 1 | 6 (50%) |
| DNA/MVA | 12 | 1 | 3 | 2 | 6 | 1 | 4 (33%) |
| Control | 4 | 0 | 1 | 0 | 3 | 0 | 1 (25%) |

### EXAMPLE 20

### Theileria parva 2nd Vaccination/Challenge

TABLE 8

| | | | Day of death | | | | | | | |
|---|---|---|---|---|---|---|---|---|---|---|
| Group | Total | Alive | 16 | 17 | 18 | 19 | 20 | 21 | 24 | 25 |
| CP/MVA | 21 | 8 | | | 3 | 4 | 2 | 4 | | |
| MVA/CP | 23 | 10 | 1 | 1 | 2 | 3 | 1 | 3 | 1 | 1 |
| PBS | 15 | 7 | 1 | | 2 | 3 | 2 | | | |

This table describes the number of cattle that died or were euthalized in each group on a given post-challenge day, i.e., the “kinetics” of the mortality after challenge in the vaccinated groups versus the non-vaccinated control group.

### EXAMPLE 21

Cattle (European and African origins) were randomized on the basis of their BoLA class I type. Vaccines were injected twice intramuscularly using a needle, the injections being 4 weeks apart. The induced immune responses were assessed for each individual antigen using IFNg ELISPOT (on both CD8+ T cells and CD4+ T cells) and determining the stimulating index (on total peripheral blood mononucleocytes). Cattle were challenged with 1×LD₁₀₀ dose of T. parva sporozoïtes

TABLE 9

| | | Cattle representing four BoLA types | | | |
|---|---|---|---|---|---|
| | Imunogen | BoLA- | | | BoLA- |
| | Tp1, Tp2, | A18 | BoLA-T2a | BoLA-A10 | T5 |
| Group | Tp4, Tp5, Tp6 | Tp1 | Tp2 | Tp4 and Tp8 | Tp5 |
| I | vCP/MVA | 3 | 3 | 3 | 3 |
| II | DNA/MVA | 3 | 3 | 3 | 3 |
| III | PBS | 1 | 1 | 1 | 1 |

### EXAMPLE 22

### Individual Kinetics of the IFNg+CD8+ T Cells Response (bovine BX223, ex-vivo stimulating antigen Tp8).

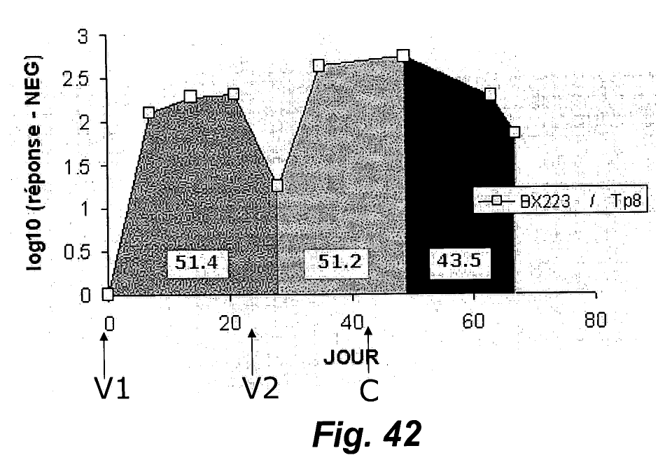

For each ELISPOT, the negative signal was substrate from the positive signal then a log10 transformation was performed. Each bovine tested was characterized by 3 areas under the curve: a priming area, a boost area, and challenge area as shown, for example, in FIG. 42. This method permitted an increase the performance of the statistical analysis by using repetitive measurement.

The frequency of IFNγ+CD8+ T cells was shown to be the most discriminative criteria. As a result, only this criterion was selected for the CPA.

### EXAMPLE 23

### Distribution of the BoLA Type Response After Ex Vivo Individual Antigen Stimulation Using PCA.

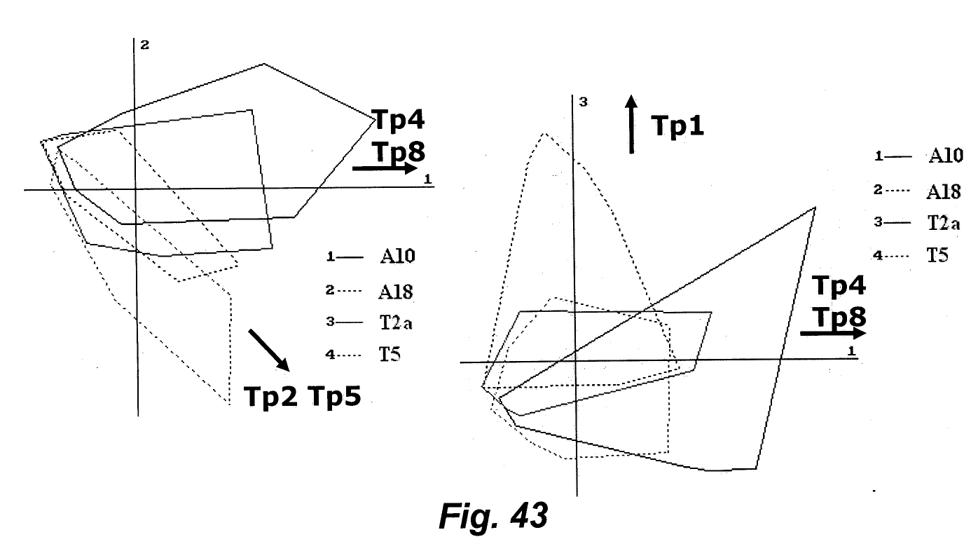

The axis 1 is characterized by Tp4 (r=0.88) and Tp8 (r=0.88) variations. The axis 2 is characterized by Tp2 (r=0.76) and Tp5 (r=0.81) variation. The axis 3 is characterized by Tp1 (r=0.89) variation. As predicted and expected and shown in FIG. 43, the antigen-specific response was restricted to the BoLA class I type: A10 (Tp4, Tp8), A18 (Tp1), T5 (Tp5, Tp2). Surprisingly, the distribution of BolA T2a is central and not influenced by the response specific for Tp2.

### EXAMPLE 24

### Protected Cattle Responded, at Least, to One Ag

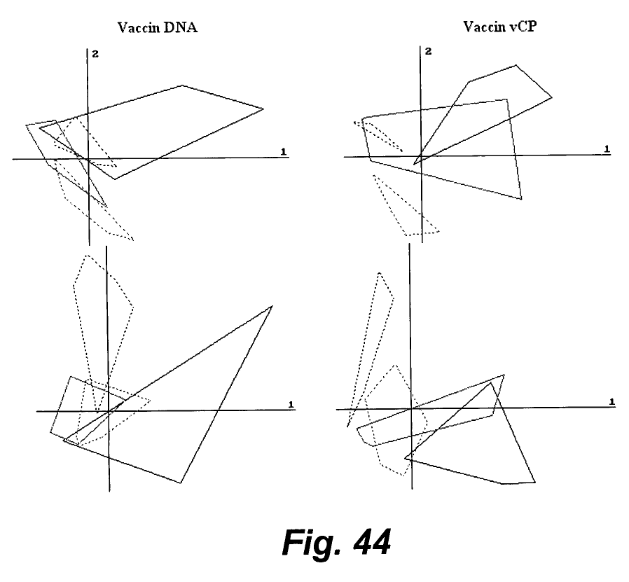

All the protected cattle responded, at least, to one Ag (BZ027). Amongst unprotected animals, some responded, whereas some individuals do not. When the vaccine regimens are compared (see, e.g., FIG. 44), based on the intensity of the IFNγ+CD8+ response after boost and challenge, they give similar profiles. The 2 tested vaccine regimens gave similar results based on INFγ+CD8+ responses

Having thus described in detailed advantageous embodiments of the present invention, it is to be understood that the invention defined by the above paragraphs is not to be limited to particle details set forth in the above description as many apparent variations thereof are possible without departing from the spirit or scope of the present invention.

What is claimed is:

1. An avipox expression vector comprising a polynucleotide encoding a Theileria parva polypeptide.

2. The avipox expression vector of claim 1, wherein the Theileria parva polypeptide is selected from the group consisting of Tp1, Tp2, Tp3, Tp4, Tp5, Tp6, Tp7 and Tp8.

3. The avipox expression vector of claim 1, wherein the Theileria parva polypeptide comprises an amino acid sequence selected from the group consisting of SEQ ID NOs: 7, 16, 26, 30, 32, 39, 44-50 and 60-64, and any fragment thereof.

4. The avipox expression vector of claim 1, wherein the avipox expression vector is an attenuated avipox expression vector.

5. The avipox expression vector of claim 4, wherein the avipox expression vector is a canarypox vector.

6. The canarypox vector of claim 5, wherein the canarypox vector is ALVAC.

7. The avipox expression vector of claim 4 wherein the avipox expression vector is a fowlpox vector.

8. The fowlpox vector of claim 7 wherein the fowlpox vector is TROVAC.

9. A formulation for delivery and expression of a Theileria parva polypeptide, wherein the formulation comprises the vector according to claim 1 and a pharmaceutically or veterinarily acceptable carrier, vehicle or excipient.

10. The formulation according to claim 9, further comprising an attenuated vaccinia virus comprising a polynucleotide having a nucleotide base sequence capable of hybridizing under stringent conditions to a nucleotide base sequence selected from the group consisting of SEQ ID NOs: 1, 6, 12, 15, 17, 19, 24, 25, 29, 33, 36, 38, 43, 51-60, or the complement thereof, and wherein the stringent hybridizing conditions are selected from (a) 40 to 45% formamide, 1 M NaCl, 1% SDS at 37° Celsius, and a wash in 0.5-1×SSC at 55 to 60° Celsius and (b) 50% formamide, 1 M NaCl, 1% SDS at 37° Celsius, and a wash in 0.1×SSC at 60 to 65° Celsius, and wherein the nucleotide base sequence has between about 75% and about 100%, between about 80% and about 100%, between about 85% and about 100%, between about 90% and about 100%, between about 95% and about 100%, between about 75% and about 90%, between about 80% and about 90%, between about 85% and about 100%, or between about 85% and about 95%, between about 85% and about 90% homology with a nucleotide sequence seletected from the group consisting of SEQ ID NOs: 1, 6, 12, 15, 17, 19, 24, 25, 29, 33, 36, 38, 43, 51-60, or the complement thereof.

11. The formulation according to claim 10, wherein the attenuated vaccinia virus isNYVAC.

12. The formulation according to claim 9, wherein the vector is a fowlpox vector.

13. The formulation according to claim 12, wherein the fowlpox vector is TROVAC.

14. The formulation according to claim 9, wherein the vector is a canarypox vector.

15. The formulation according to claim 14, wherein the canarypox vector is ALVAC.

16. A method of delivering a Theileria parva antigen to an animal, comprising administering the formulation according to claim 9 to the animal.

17. A method of eliciting an immune response against a strain of Theileria in an animal, comprising administering a formulation according to claim 9 and in an effective amount for eliciting an immune response.

18. A method of inducing an immunological or protective response against a strain of Theileria in an animal, comprising administering to the animal an effective amount of the formulation according to claim 9.

19. A method of eliciting an immune response against a strain of Theileria in an animal, comprising administering a composition comprising a cell, wherein the cell comprises the vector according to claim 1 in an effective amount for eliciting an immune response.

20. A kit for performing a method of inducing an immunological or protective response against a strain of Theileria in an animal comprising the vector according to claim 1 and instructions for performing the method of delivery and expression of the vector in the animal.

21. A kit for performing a method of inducing an immunological or protective response against a strain of Theileria in an animal comprising the formulation according to claim 9 and instructions for performing the method of delivery and expression of the vector in the animal.
